(19) United States
(12) Patent Application Publication
(10) Pub. No.: US 2023/0257734 A1
(43) Pub. Date: Aug. 17, 2023

(54) Activated reporter protein for the detection of infection in a biological sample

(71) Applicants: INSTITUT PASTEUR, PARIS (FR); CENTRE NATIONAL DE LA RECHERCHE SCIENTIFIQUE - CNRS, PARIS (FR)

(72) Inventors: Bjorn MEYER, PARIS (FR); Marco VIGNUZZI, PARIS (FR)

(21) Appl. No.: 18/003,263

(22) PCT Filed: Jul. 2, 2021
(86) PCT No.: PCT/EP2021/068402

§ 371 Date: Dec. 23, 2022

(30) Foreign Application Priority Data
Jul. 3, 2020 (EP) 20305758.3

(51) Int. Cl. C12N 15/10; C12Q 1/37

(52) U.S. Cl. CPC C12N 15/1055; C12Q 1/37

(57) ABSTRACT

The invention relates to novel means and processes for the detection of a virus in a biological sample comprising cells infected by the virus. In particular, the invention relates to a fluorescent reporter protein designed as a recombinant inactive form of flipGFP suitable for specific activation by viral components in particular by viral proteins, such as viral protease, wherein the viral component recognizes a cleavage site inserted in the recombinant flipGFP. The fluorescent reporter protein is suitable for use in an in vitro method of detection of virus infection in a biological sample when the virus is related to the viral components activating the inactive form of flipGFP into an active fluorescent flipGFP in a biological sample, especially a sample comprising cells, in particular unaltered cells.

The invention relates to novel means and processes for the detection of a virus in a biological sample comprising cells infected by the virus. In particular, the invention relates to a reporter protein designed for specific activation by viral components, in particular by viral proteins, such as viral protease, suitable for the detection of virus associated with such viral components, in a biological sample comprising cells sensitive to virus infection or rendered sensitive to virus infection.

The invention hence relates to an in vitro method of detecting a virus or a virus infection in a biological sample, in particular in a sample that is regarded as intact, i.e., that does not require treatment to get access to the virus or the virus components, including to the viral genome.

Currently the detection of viruses relies on either visual signs of infection, such as cytopathic effects (CPE) when a virus infection results in cell death towards the end of the infection cycle, or it relies on methods that do not allow for conditions that keep the sample intact, referred to as live conditions. Such methods include, but are not limited to, plaque assays, TCID50s, PCR-based methods or sequencing methods.

With a view to design detection means based on fluorescence of a reporter protein, wherein the detection means would be suitable to enable detection of any type of viruses independently of their replication pathway in host cells and without interfering with the expression of viral components and integrity of the viral genome, the inventors have considered providing altered fluorescent proteins that would be activated in a biological sample in the presence of the virus to be detected or its viral proteins. In a preferred aspect the invention makes use of the indirect detection of the presence of proteins involved in achieving proper replication and assembly of viral particles, or mediating particle disassembly upon virus entry into a newly infected cell, especially makes use of detection of viral proteases in the assayed biological sample. Besides their biological function in the assembly and disassembly of virus particles, proteases additionally give rise to proteolytic cleavage in cells infected by the virus.

Accordingly, the invention relies on genetically modified fluorescent proteins suitable for use as reporter proteins for infection of a sample by a determined virus, wherein the modification in the protein enables the fluorescent protein to become a sensor for a determined viral protease, especially of a protease of a virus infecting human or animals (human virus or animal virus). Such fluorescent proteins have been prepared for the purpose of the invention by modifying known split fluorescent proteins. In particular, the genetically modified fluorescent protein suitable for use as reporter protein for infection of a sample by a determined virus is engineered in such a way that assaying the viral infection of a biological sample does not require prior purification of the viral protease interacting with the genetically modified fluorescent reporter protein. Otherwise stated the genetically modified fluorescent protein suitable for use as reporter protein for infection by a determined virus advantageously enables to carry out the detection when viral proteins other than the sought protease are present in the detected sample.

Systems derived from Spilt Fluorescent Protein (FP) are built according to the invention by engineering the fluorescent protein in order to confer to the fluorescent protein a conditional reporter activity that is based on occurrence of determined specific cleavage events in the engineered fluorescent protein that prove necessary to allow the reconstitution of a functional fluorescent FP. According to the invention, cleavage events in the sequence of the engineered FP are elicited by the internalization or the expression in a host cell expressing or transformed with the engineered FP system, of a protein of a pathogen (such as a viral protein, a bacterial protein or a parasitic protein), especially a protease, more particularly a viral protease. In a particular embodiment, a pathogen protease such as a viral protease of a human or animal virus that specifically targets the cleavage site engineered in the FP system is present in the hot cell of the virus expressing this protease and activates the FP system to allow it to fluoresce. In another embodiment, the engineered FP system is expressed in a host cell that is contacted with a sample suspected of containing the virus. Advantageously the invention enables to detect virus infection in a sample obtained from a subject without requiring purification of the virus protease for contacting it with the engineered FP system and assessing infection of the sample.

According to a particular aspect, the invention relates to a process for the detection of an infection, especially a viral infection in a biological sample previously taken from a human subject or an animal, wherein the process comprises the step of transforming cells of the sample with a FP system comprising a fluorescent reporter protein that would be able to become fluorescent in the cell when its protease cleavage site is targeted and cleaved by a determined viral protease present in the cell and detecting whether fluorescence of the reporter protein is present.

According to another aspect, the invention relates to a process for the detection of an infection, especially a viral infection in a biological sample previously taken from a human subject or an animal, wherein the process comprises the step of providing cells expressing a FP system comprising a fluorescent reporter protein that would be able to become fluorescent in the cell when its protease cleavage site is targeted and cleaved by a determined viral protease present in an assayed biological sample contacted with said cells and detecting whether fluorescence of the reporter protein is present.

Applied to the detection of virus infection, the process of the invention may be efficient as it would detect virus presence a few hours after infection, in a specific and sensitive manner and would not require treating or denaturing the cell sample to isolate the virus material or its proteins.

As an example of the FP system suitable to carry out the invention an inactive form of split GFP, i.e., flipGFP, has been chosen to illustrate the preparation of an engineered novel flipGFP expressed in a host cell where it exhibits fluorescence under conditional elicitation by viral proteases. FlipGFP according to the invention is accordingly an illustration of a particular protease sensor wherein the protease originates from a pathogen, in particular a virus.

GFP (also designated split-GFP) was cloned from Aequorea Victoria and has been shown to comprise 238 amino acids (with a cut-off site at position 214/215) that form a barrel structure with 11 beta strands (β-strands) with a central alpha helix (α-helix). The sequence of the GFP has been published (Tsien R.Y. Annu Rev. Biochem, 1998, 67, 509-44) and is available under accession number Q6YGZ20 (uniprot). GFP can be split into three parts. One part contains beta-strands 1 to 9 and the alpha helix (designated β1-9 or GFP1-9 -amino acids 1-193), a second part contains the 10^{th} β-strand (β10 - amino acids 194-212) and the third part contains the 11^{th} β-strand (β11 - amino acids 213-233). β1-9 contains three amino acids (Ser65, Tyr66, Gly67) that undergo cyclisation, dehydration and oxidation during maturation to form the chromophore whereas β11 contains the highly conserved amino acid residue Glu222 that catalyzes chromophore maturation (Sniegowski, J.A. J.Biol. Chem 2005, 280 (28), 26248). The activation of the fluorescence is elicited when β10 and β11 are linked together or are in close proximity and then bind to β1-9 that leads to the development of fluorescence within several tens of minutes (Cabantous, S. et al. Sci. Rep. 2013, 3, 2854). In the native structure of GFP, β10-11 forms an anti-parallel beta-strands which fits well within the rest of the structure, β1-9. The structure of the GFP was flipped in order to achieve parallel β10-11 structure so that it did not spontaneously fit β1-9 but rather required a signal that was chosen to be provided by protease cleavage at a specific protease cleavage site inserted into the sequence of β10-11: cleavage allowed flipping back (or reverting orientation) of β11 strand in an anti-parallel structure with β1, thereby enabling self-assembly with β1-9 and increase in fluorescence. The thus engineered reporter was named flipGFP and considered an inactive form of split-GFP that needed an external signal to become activated. It was used for imaging spatiotemporal dynamics of apoptosis in Zebrafish and in Drosophila (Zhang Q. et al J. Am. Chem. Soc., 2019, 141, 4526-4530). avGFP has a maximum excitation peak at 396-398 nm (corresponding to the neutral state of Tyr66 in the chromophore) and a lower excitation peak at 476-478 nm (corresponding to the deprotonated anionic state of Tyr66 and the emission peak is at 510 nm (Pedelacq J.D. and Cabantous S. Int. J. Mol. Sci. 2019, 20, 3479).

It has been observed in the art that GFP fused to other proteins in order to report on their activity may exhibit drawbacks such as folding defects that impact its fluorescence and reporter capability. Accordingly, improved GFP such as superfolder GFP variants obtained by mutation of specific positions have been developed the folding of which is not affected by fusion to poorly folded proteins (Pedelacq J.D. et al Nature Biotechnology 2006, 24,1, 79-88). Such improved variants of GFP may be used in the invention.

In a first aspect, the invention relates to a nucleic acid construct which comprises (a) a recombinant transgene that encodes a recombinant inactive form of a fluorescent reporter protein, and a cleavage site for a viral protease, and (b) a nucleic acid coding for a detectable expression control protein, wherein the nucleic acid sequences of (a) and (b) are operably assembled in said operon under the control of a single promoter and optionally of additional control sequence(s) for transcription and/or translation and wherein the nucleic acid sequences of (a) and (b) are optionally separated by the sequence of a polyprotein separating site such as the sequence of the separating 2A-peptide originating from Thosea asigna virus capsid.

In a particular embodiment, the invention relates to a nucleic acid construct which comprises an operon wherein the operon comprises (a) a recombinant transgene that encodes a recombinant inactive form of a fluorescent reporter protein, and a cleavage site for a viral protease, and (b) a nucleic acid coding for a detectable expression control protein, wherein the nucleic acid sequences of (a) and (b) are operably assembled in said operon under the control of a single promoter and optionally of additional control sequence(s) for transcription and/or translation and wherein the nucleic acid sequences of (a) and (b) are optionally separated by the sequence of a polyprotein separating site such as the sequence of the separating 2A-peptide originating from Thosea asigna virus capsid.

A nucleic acid construct of the invention is in particular a DNA construct, especially a double-stranded DNA construct. It may in particular be a cDNA molecule.

In a particular embodiment, the nucleic acid coding for a detectable expression control protein of (b) is interposed within sequence encoding structural domains of the sequence of the active form of a fluorescent reporter protein. According to this embodiment, the sequence encoding structural domains of the active form of a fluorescent reporter protein is interrupted by the sequence encoding the detectable expression control protein.

The expression “fluorescent protein” (FP) in particular a “fluorescent reporter protein” or the likes according to the invention means a protein that is able to fluoresce in a cell when expressed in proper conditions that enable maturation of its chromophore (also designated fluorophore). In the context of the invention, a first category of fluorescent proteins encompasses a fluorescent reporter protein that becomes active to fluoresce when it assembles as an active form of the protein. “Assembly” (assembled) means that the structural domains of the protein provide for a structure that reproduces the wild-type or the native conformation of the protein that allows maturation of the chromophore. Assembly may require that multiple structural domains in the proteins be correctly assembled. A second category of fluorescent proteins for use according to the invention encompasses expression control proteins that fluoresce as soon as the nucleic acid construct of the invention is expressed. Such fluorescent proteins for control of expression are well known from the person skilled in the art.

The expression “active fluorescent reporter protein” is used interchangeably with the terms “fluorescent reporter protein” and designates the fluorescent protein that has adopted a functional conformation after specific site cleavage of the inactive fluorescent protein following contact or interaction with a suitable protease recognizing said specific site, according to the invention.

The expression “inactive fluorescent reporter protein” relates to the fluorescent reporter protein the amino sequence of which has been modified such that (i) when expressed by the nucleic acid construct of the invention, its structure domains cannot assemble as in the wild type or the native form of the fluorescent protein to allow maturation of its chromophore and that (ii) it contains an amino acid sequence of a cleavage site for a protease in accordance with the disclosure provided herein. As a consequence of the presence of this cleavage site, upon cleavage the inactive fluorescent protein reconstitutes its wild type or native structure and fluoresce thereby becoming active or functional. Cleavage involves conditions disclosed herein that are related to enabling interaction, especially contact, between the inactive fluorescent protein and a protease targeting the cleavage site wherein such interaction, especially contact, is performed with the biological sample containing the protease, e.g., without denaturation of the sample or without separation of the protease from the sample. According to this embodiment, the insertion of the nucleic acid coding for a detectable expression control protein of (b) in the transgene does not interfere with the sequence of the protease cleavage site.

Accordingly, the invention relates to a particular nucleic acid construct wherein the recombinant transgene encoding the inactive form of the fluorescent reporter protein comprises a nucleotide sequence of an altered form of the Open Reading Frame that encodes the active form of the fluorescent reporter protein and wherein said alteration in the ORF comprises switching position in the ORF of at least one nucleotide sequence encoding specific structure domains of the active form of the fluorescent protein to prevent assembly of the expressed structure domains as a functional protein enabling maturation of the chromophore and wherein the nucleotide sequence encoding the inactive form of the fluorescent reporter protein additionally comprises a nucleotide sequence encoding a cleavage site for a determined protease. In a particular embodiment, the altered form of the ORF of the active form of the fluorescent reporter protein is a nucleotide sequence wherein the polynucleotide encoding at least two specific structure domains of the fluorescent reporter protein is separated from its naturally contiguous sequence in the active form of the fluorescent protein and optionally is permuted in the obtained transgene with respect to its location with regard to the other structure domains in the active form of the fluorescent reporter protein.

In a particular embodiment, when the nucleic acid coding for a detectable expression control protein (of (b) above) is interposed between nucleic acid sequence of structural domains of the sequence in the inactive form of the fluorescent reporter protein, it does not interfere with the capability of the sequence as a whole to encode the structural domains, in particular it is inserted in a site outside all sequences encoding the structure domains of the active form of a fluorescent reporter protein. In such embodiment, the nucleic acid coding for a detectable expression control protein of (b) separates the sequence encoding the structure domains of the active form of a fluorescent reporter protein in at least two, in particular two, polynucleotides. In particular, the interposition of the nucleic acid coding for a detectable expression control protein prevents the expression of the active form of a fluorescent reporter protein.

The expression “host cell” refers to a cell that is present in the biological sample either naturally or after introduction by available technical means in order to enable suitable conditions for the expression or the display of the inactive and the active forms of the fluorescent reporter protein. In a particular embodiment, the host cell is a cell that can be infected by the pathogen targeting the cleavage site in the inactive fluorescent protein. Especially, the host cell is a cell that can be infected by a human or an animal virus. In a particular embodiment, the host cell may be a cell line transformed (especially transected or transduced) with the nucleic acid construct as disclosed herein.

The expression “operably linked” or “operatively/operably linked” “operatively/operably assembled” or “operatively/operably cloned”, refers to the functional cloning, or insertion, of polynucleotides within the nucleic acid construct of the invention. In a particular embodiment, the nucleic acid sequences encoding the flipGFP switched domains (designated flipGFP10-11 in the figures) of the inactive form of the FP are cloned in a different open reading frame from the open reading frame of the sequence encoding the cleavage site for the viral protease. The sequences encoding the other polypeptides of the operon or of the transgene, including e.g. the separating peptide and the expression control protein are cloned in the same reading frame as the reading frame of the nucleic acid sequences encoding the flipGFP switched domains (designated flipGFP10-11 in the figures) of the inactive form of the FP. The polynucleotides are operably linked when they can be expressed from the nucleic acid construct, as recombinant polypeptides such as in the inactive form of the fluorescent reporter protein or as individualized polypeptides such as the expression control protein.

The expression of the encoded fluorescent reporter protein and the expression of the control protein may be under the control of the same promoter that may be any known promoter, including CMV promoter as exemplified herein. In a particular embodiment, the promoter of the transgene or of the operon is active in cells selected from the group of prokaryotic cells, in particular in bacterial cells or in archaeal cells, and/or is active in eukaryotic cells, in particular in mammalian cells or in insect cells.

A fluorescent protein according to this embodiment is a protein that comprises multiple structural domains that need to be assembled and folded in native conformation to allow fluorescence to develop as a result of providing a mature chromophore that has acquired visible absorbance of light and fluorescence.

In a particular embodiment, the nucleic acid construct is such that the active fluorescent reporter protein is an active flipGFP and the inactive fluorescent reporter protein is an inactive flipGFP.

A particular embodiment of the fluorescent protein used in the invention is the GFP as disclosed above based on the protein originally identified in Aeoquorea jellyfish. Beyond the disclosed GFP having the polypeptide sequence disclosed by Tsien R.Y, various isoforms of the sequence of Aeoquorea GFP have been identified in the art. They may interchangeably be considered for the purpose of the novel flipGFP construction according to the invention. Besides, GFP from other organisms have also been identified such as GFP originating from anthozoa Renilla, or GFP of other coelenterates such as Obelia or Phialidium. In addition, mutated GFP having improved properties when used as reporter protein have been made available in the art, including the proteins as disclosed in FIG. 1. All these forms of GFP may be used to derive the novel flipGFP construct for use in the invention.

“switching position” relative to the ORF of at least one structure domain of the fluorescent reporter protein to achieve an inactive form of the protein involves or is a transfer of the nucleotide sequence of at least one structure domain of the fluorescent reporter protein at a location in the transgene that alters the ORF of the active form of the fluorescent protein. Otherwise stated the switch breaks the structural connection between the nucleotide sequences of the switched structure domain(s) of the fluorescent protein. In a particular embodiment, a switch in position accordingly may involve permutation (“switching by permutation”) of the nucleotide sequence coding for at least two structure domains of the protein or permutation of sequences encoding contiguous groups of domains in the ORF coding for the protein, in the nucleic acid construct of the invention. This is illustrated by the nucleotide sequence of the flipGFP where the nucleotide sequences encoding respectively the GFP1-9 and GFP10-11 domains are permutated in the nucleic acid construct of the invention. In another particular embodiment, the switch in position may result from the recombination of the ORF of the active form of the fluorescent protein by insertion or interposition of a foreign nucleotide sequence (“switching by insertion”) with respect to said ORF, such as a foreign nucleotide sequence comprising or consisting of a sequence encoding an expression control protein (as illustrated with the inserted T2A sequences and mCherry protein). Accordingly, the switched structure domain(s) of the fluorescent reporter protein (in particular the switched GFP10-11 domains of the flipGFP) may be contained in the transgene, including according to an embodiment where the transgene is recombined with the nucleic acid encoding the detectable expression control protein and optionally additional sequences suitable for the expression of a detectable fluorescent reporter protein after protease cleavage.

In a further particular embodiment of a nucleic acid construct according to the invention, the recombinant transgene comprises from 5′-end to 3′-end polynucleotides encoding the beta10 strand of flipGFP, a linker having from 3 to 15, in particular about 10, amino acid residues, the E5 domain of flipGFP, the beta11 strand of flipGFP, the cleavage site of the viral protease and the K5 domain of the flipGFP, the sequence of a polyprotein separating site such as the sequence of the separating 2A-peptide originating from Thosea asigna virus capsid, a polynucleotide encoding the beta1-9 strand of flipGFP, wherein these polynucleotides together encode the inactive form of the recombinant flipGFP with the viral protease cleavage site.

In a further particular embodiment, the nucleic acid construct according to the invention comprises from 5′-end to 3′-end, polynucleotides encoding the beta1-9 strand of flipGFP, the sequence of a polyprotein separating site such as the sequence of the separating 2A-peptide originating from Thosea asigna virus capsid, the nucleic acid coding for a detectable expression control protein, polynucleotides encoding the beta10 strand of flipGFP, a linker having from 3 to 15, in particular about 10, amino acid residues, the E5 domain of flipGFP, the beta11 strand of flipGFP, the cleavage site of the viral protease and the K5 domain of the flipGFP, wherein these polynucleotides together encode the inactive form of the recombinant flipGFP with the viral protease cleavage site and the detectable expression control protein

Amino acid sequence of a 2A-peptide originating from Thosea asigna virus capsid may be the sequence of SEQ ID No.147.

In an embodiment of a nucleic acid construct according to the invention, the transgene further comprises upstream from the sequence encoding the inactive fluorescent reporter protein in particular the inactive flipGFP, a sequence encoding a signal peptide, in particular a signal peptide for retention of the expressed polypeptides in the endoplasmic reticulum (ER retention signal), or a signal peptide for targeting the expressed polypeptides into the ER membrane or a signal peptide for targeting the expressed polypeptides to the cell membrane (membrane targeting signal), especially a Cytochrome P450 ER retention signal of sequence MDPVVVLGLCLSCLLLLSLWQSHGGGK (SEQ ID No.105) or a membrane targeting signal of sequence MGCCFSKT (SEQ ID No.107).

In another embodiment of a nucleic acid construct according to the invention, the operon or the transgene, further comprises operably associated with the sequence encoding the inactive fluorescent reporter protein, in particular downstream from said sequence, a signal peptide, in particular a signal peptide for retention of the expressed part of the fluorescent reporter polypeptide associated thereto in the endoplasmic reticulum (ER retention signal) and/or a peptide for targeting the expressed polypeptides to the ER membrane or the cell membrane (such as a transmembrane targeting signal). The above cited sequences encoding the particular signal peptides may be used. Alternatively, nucleic acid sequences encoding a peptide containing a transmembrane domain enabling retention into the ER membrane may be suitable for insertion into the construct of the invention. Examples of such sequences are provided herein such as the sequence that encodes the HCV Core TM peptide and is the sequence from position 4645 to position 4821 in SEQ ID No.150 (SEQ ID No.152) or the sequence that encodes the HIV Vpu peptide which is the sequence from position 4645 to position 4944 in SEQ ID No.151 (SEQ ID No.153). These HCV Core TM peptide and HIV Vpu peptide may in particular be used in association with the inactive flipGFP. These sequences allow retention into the ER membrane.

As an illustration to carry out the invention, the polynucleotide encoding the signal peptide may contain or consist of the sequence of ATGGACCCCGTGGTGGTGCTGGGCCTGTGCCTGAGCTGCCTGCTGCTGCTGAGCCTGT GGAAGCAGAGCCACGGCGGCGGCAAG (SEQ ID No.104) encoding the Cytochrome P450 ER retention signal peptide or may contain or consist of the sequence of ATGGGCTGCTGCTTCAGCAAGACC (SEQ ID No.106) encoding the membrane targeting signal peptide. Alternatively, the polynucleotide encoding the peptide in particular a transmembrane peptide contains or consists of the sequence of SEQ ID No.152 encoding the HCV Core TM peptide, or the sequence of SEQ ID No.153 encoding the HIV Vpu peptide for retention into the ER membrane or for retention in the ER.

Any alternative signal peptide known to the person skilled in the art and suitable to address the expressed polypeptides to a specific cellular compartment, especially to the cell membrane or to the endoplasmic reticulum, may be used to perform the invention. The sequence encoding a signal peptide in the nucleic acid construct of the invention enables expression of the polypeptides in a specific cell compartment and, as a consequence, enables their accessibility for the protease of the virus when contacted with the assayed sample.

In a particular embodiment, the operon of the transgene additionally contains a sequence encoding a polypeptide suitable for proper folding of the expressed fluorescent reporter protein and the expression control protein, independently of each other. Such sequence may be the sequence of a hinge of an immunoglobulin, in particular of an IgG such as illustrated in FIGS. 11 or 12 and in the sequence of SEQ ID No.150 or SEQ ID No.151. This sequence may advantageously be located 5′ from the sequence encoding the fluorescent reporter protein in particular 5′ from the last fragment of this sequence in the transgene and before the sequence encoding the signal peptide when this sequence if provided downstream of the coding sequences of the construct. Such sequence shall not interfere with the integrity of the sequence encoding the cleavage site.

As mentioned hereabove a nucleic acid construct according to the invention encodes an expression control protein.

The expression “expression control protein” defines a protein that may be detectable by any means in particular by fluorescence in conditions that would prevent detrimental, overlapping between fluorescence of the expression control protein and fluorescence of the fluorescent reporter protein. In particular, the fluorescence of expression control protein and of the FP may take place in different light ranges or may be of a different color. The expression control protein may be used to ascertain the expression of the fluorescent reporter protein in conditions where fluorescence of the sample may interfere with the expected fluorescence of the FP. Accordingly, expression of the expression control protein may ensure that the detected fluorescence alleged to arise from the FP is not merely a background fluorescence.

The expression control protein may be a fluorescent protein with a fluorophore of a color that is different from the color of the fluorescent reporter protein. In particular, the expression control protein may be an mCherry protein or mTurquoise protein or cyan fluorescent protein (ECFP), yellow fluorescent protein such as mVenus, in particular the nucleic acid construct contains the polynucleotide of SEQ ID No. 146 coding for mCherry.

In the nucleic acid construct of the invention, the sequence encoding the cleavage site in the fluorescent reporter protein should be inserted in the sequence encoding the inactive form of the fluorescent protein at a position of the sequence that would not hamper the maturation of the chromophore in the expressed activated protein said position being further chosen to enable restoration of the active conformation of the fluorescent protein after cleavage. In a particular embodiment, insertion site for the cleavage site sequence may be at the junction of structural domains. Illustration of the insertion position of the cleavage site is especially provided for the flipGFP and thus gives guidance for the person skilled in the art to derive alternative embodiments.

The expression “cleavage site” refers to a peptide or polypeptide functional as a site to allow cleavage of its amino acid sequence when recognized by a specific enzyme, i.e. a pathogen protease, in particular a viral protease. In a particular embodiment, the cleavage site is said to be “specific” when it is efficiently recognized and cleaved by a protease of a single virus or by a protease shared by members of a determined family or group of pathogens such as a class or a group of viruses. A cleavage site has a wild type sequence originating from a known pathogen, in particular a known virus of a group of viruses or has a modified sequence, such as a consensus sequence suitable to improve recognition and cleavage by the relevant protease of several pathogens in a determined group of pathogens, especially viruses. The cleavage site comprises amino acid residues surrounding the precise residues where the cleavage takes place in the target sequence for the protease. In a particular embodiment, the cleavage site comprises more than 8 amino acid residues, in particular more than 10, especially 12 amino acid residues. In a particular embodiment, the number of amino acid residues is similar in the N-terminal and C-terminal parts of the sequence relative to the location of the residue where the cleavage takes place. In a particular embodiment, the amino acid sequence of the cleavage site may be derived from the native sequence targeted by the selected protease in a viral strain. In another particular embodiment, variation in the amino acid residues relative to the native sequence may be performed.

In a particular embodiment of the nucleic acid construct, the cleavage site contained in the inactive fluorescent protein is recognized by proteases of a determined virus family or genus selected in the group of Alphaviruses, Coronaviruses, Enteroviruses, Retroviruses and Flaviviruses.

These families of viruses are of particular interest for the purpose of early detection of infection. Families of viruses such as these cited above or sub-families thereof may share features including similar sequences for cleavage site of proteases, such similarity being sufficient to allow cross-recognition by proteases of the various virus members of the family or the sub-family. Shared cleavage sites for proteases in a family or sub-family or even in a genus may allow the detection of viruses at the level of their family, sub-family or respectively genus and accordingly be in particular useful to obtain a primary indication of the cause of an infection in a subject especially when it can be obtained at an early stage of the infection.

The inventors have in particular selected sequences of peptides or polypeptides that bear the cleavage site for a protease active in viruses belonging to these families.

Accordingly, in a second aspect, the invention concerns a polynucleotide suitable for the preparation of the nucleic acid constructs of the invention, wherein such polynucleotide comprises or consists in the sequence coding for an inactive fluorescent reporter protein wherein the sequence insert for the cleavage site for a protease is selected among cleavage site recognized by proteases of a determined virus family selected in the group of Alphaviruses, Coronaviruses, Enteroviruses, Retroviruses and Flaviviruses. Alternatively, the polynucleotide encoding the cleavage site may be selected for its capability to be recognized by the protease of a specific virus or a specific virus strain.

According to a particular embodiment of the nucleic acid construct, the nucleic acid sequence for the protease cleavage site encodes an amino acid sequence selected from the group of: ITTLGKFGQ (SEQ ID No.126) for the Enterovirus 2A protease, EALFQGPK (SEQ ID No.127) or SYFASEQGEIQWV (SEQ ID No.128) for the Enterovirus 3C protease, RAGAYIFS (SEQ ID No.129) for Alphaviruses, RELNGGAYTRYV (SEQ ID No.130),FTLKGGAPTKVT (SEQ ID No.131), IALKGGKIVNNW (SEQ ID No.132), TSAVLQSGFRKM (SEQ ID No.133), KVATVQSKMSDV (SEQ ID No.134), SAVKLQNNELSP (SEQ ID No.135), ATVRLQAGNATE (SEQ ID No.136), REPMLQSADAQS (SEQ ID No.137) and SGVTFQSAVKRT (SEQ ID No.138) for SARS-CoV-2 coronavirus, SGVTFQGKFKK (SEQ ID No.139) for SARS virus coronavirus, YAKRGGVF (SEQ ID No140) for Flaviviruses, and more particularly KERKRRGADTSI (SEQ ID No.141), TRSGKRSWPPSE (SEQ ID No. 142), EPEKQRSPQDNQ (SEQ ID No.143), GLVKRRGGGTGE (SEQ ID No.144) for ZIKA viruses.

The above cited peptide forming cleavage site for the SARS-CoV-2 coronavirus are specific for cleavage by a protease of the virus in particular by the main protease of the virus also designated M^{pro,}, nsp5 or 3CL^{pro} that requires the minimal LQ consensus sequence for cleavage (after the Q residue) or alternatively by the protease designated nsp3.

In another particular embodiment of the nucleic acid construct, the polynucleotide encoding the viral protease cleavage site consists of a polynucleotide selected from the group of:: GAGGACAGGGCCGGCGCCGGCATCATCGAGACCCCC for CHKV-1 (SEQ ID No.108) or GCCACCAGGGCCGGCTGCGCCCCCAGCTACAGGGTG for CHKV-2 (SEQ ID No.109) or CTGGACAGGGCCGGCGGCTACATCTTCAGCAGCGAC for CHKV-3 (SEQ ID No.110) or, ATCACCACTCTTGGGAAATTTGGACAA for EV71_2A (SEQ ID No.111) or AGCTACTTCGCCAGCGAGCAGGGCGAGATCCAGTGGGTG for EV71_3C (SEQ ID No.112) or, AGGGAGCTGAACGGCGGCGCCTACACCAGGTACGTG for SARSCoV2_1 (SEQ ID No.113) or TTCACCCTGAAGGGCGGCGCCCCCACCAAGGTGACC for SARSCoV2_2 (SEQ ID No.114) or ATCGCCCTGAAGGGCGGCAAGATCGTGAACAACTGG for SARSCoV2_3 (SEQ ID No.115) or ACCAGCGCCGTGCTGCAGAGCGGCTTCAGGAAGATG for SARSCoV2_4 (SEQ ID No.116) or AGCGGCGTGACCTTCCAGAGCGCCGTGAAGAGGACC for SARSCoV2_5 (SEQ ID No.117) or AAGGTGGCCACCGTGCAGAGCAAGATGAGCGACGTG for SARSCoV2_6 (SEQ ID No.118) or AGCGCCGTGAAGCTGCAGAACAACGAGCTGAGCCCC for SARSCoV2_7 (SEQ ID No.119) or GCCACCGTGAGGCTGCAGGCCGGCAACGCCACCGAG for SARSCoV2_8 (SEQ ID No.120) or AGGGAGCCCATGCTGCAGAGCGCCGACGCCCAGAGC for SARSCoV2_9 (SEQ ID No.121) or, AAGGAGAGGAAGAGGAGGGGCGCCGACACCAGCATC for ZIKV_1 (SEQ ID No.122) or ACCAGGAGCGGCAAGAGGAGCTGGCCCCCCAGCGAG for ZIKV_2 (SEQ ID No.123) or GAGCCCGAGAAGCAGAGGAGCCCCCAGGACAACCAG for ZIKV_3 (SEQ ID No.124) or GGCCTGGTGAAGAGGAGGGGCGGCGGCACCGGCGAG for ZIKV_4 (SEQ ID No.125).

In a particular embodiment, the nucleic acid construct comprises a polynucleotide that encodes a flipGFP of the sequence of SEQ ID No.145 recombined with one of these nucleotide sequences coding for one or more of the above cleavage sites. In a particular embodiment, the sequence encoding the one or multiple cleavage sites for the viral protease is inserted at position 262 in said sequence.

In another embodiment, the novel flipGFP is encoded by the sequence of SEQ ID No.145 and the sequence of the cleavage site for the protease, in particular a sequence chosen among the illustrated sequences herein for viral cleavage sites is inserted at position 262 in said sequence.

According to a particular embodiment, the polynucleotide for insertion in the recombinant transgene to encode the recombinant inactive recombinant flipGFP is selected from the group of SEQ ID No. 47 to SEQ ID No.100 respectively for flipGFP_CHKV_1, flipGFP_CHKV_2, flipGFP_ER_CHKV_1, flipGFP_ER_CHKV_2, flipGFP_ER_CHKV_3, flipGFP_ER_EV71_2A, flipGFP_ER_EV71_3C, flipGFP_ER_SARSCoV2_1, flipGFP_ER_SARSCoV2_2, flipGFP_ER_SARSCoV2_3, flipGFP_ER_SARSCoV2_4, flipGFP_ER_SARSCoV2_5, flipGFP_ER_SARSCoV2_6, flipGFP_ER_SARSCoV2_7, flipGFP_ER_SARSCoV2_8, flipGFP_ER_SARSCoV2_9, flipGFP_ER_ZIKV_1, flipGFP_ER_ZIKV_2, flipGFP_ER_ZIKV_3, flipGFP_ER_ZIKV_4, flipGFP_EV71_2A, flipGFP_EV71_3C, flipGFP_Membrane_CHKV_1, flipGFP_Membrane_CHKV_2, flipGFP_Membrane_CHKV_3, flipGFP_Membrane_EV71_2A, flipGFP_Membrane_EV71_3C, flipGFP_Membrane_SARSCoV2_1, flipGFP_Membrane_SARSCoV2_2, flipGFP_Membrane_SARSCoV2_3, flipGFP_Membrane_SARSCoV2_4, flipGFP_Membrane_SARSCoV2_5, flipGFP_Membrane_SARSCoV2_6, flipGFP_Membrane_SARSCoV2_7, flipGFP_Membrane_SARSCoV2_8, flipGFP_Membrane_SARSCoV2_9, flipGFP_Membrane_ZIKV_1, flipGFP_Membrane_ZIKV_2, flipGFP_Membrane_ZIKV_3, flipGFP_Membrane_ZIKV_4, flipGFP_SARSCoV2_1, flipGFP_SARSCoV2_2, flipGFP_SARSCoV2_3, flipGFP_SARSCoV2_4, flipGFP_SARSCoV2_5, flipGFP_SARSCoV2_6, flipGFP_SARSCoV2_7, flipGFP_SARSCoV2_8, flipGFP_SARSCoV2_9, flipGFP_ZIKV_1, flipGFP_ZIKV_2, flipGFP_ZIKV_3, flipGFP_ZIKV_4, flipGFP-SARS2-HCVCore also designated flipGFP-SARS2-10-HCVCore (contained in SEQ ID No.150), and flipGFP-SARS2-HIVVpu also designated flipGFP-SARS2-10-HIVVpu (contained in SEQ ID No.151).

The invention also relates to each of the above nucleic acid sequences encoding a recombinant inactive flipGFP as such.

The invention also relates to a set of at least two nucleic acid constructs as defined herein, or of recombinant nucleic acids as defined herein or of polynucleotides as defined herein.

The invention also concerns a transformation vector which comprises a nucleic acid construct or a recombinant nucleic acid or a polynucleotide, in particular a vector which is a plasmid for transfection, especially a lentiviral vector plasmid.

In a particular embodiment, when the nucleic acid construct is a lentiviral vector plasmid, the polynucleotide sequences are operably linked within the cDNA encoding the genome of the lentivirus vector. Accordingly, in the context of the invention the nucleic acid sequence of the polynucleotide encoding a fluorescent reporter protein is fused within the sequence of the plasmid comprising the genome of the lentiviral vector in such a manner that the construct enables expression of infectious recombinant viral particles (in particular pseudotyped recombinant viral particles) of the lentiviral vector and enables such recombinant lentiviral particles to express the fluorescent protein in appropriate conditions. The nucleic acid construct accordingly comprises the nucleotide sequence necessary for replication of the recombinant vector genome and for the expression of the encoded proteins.

The expression “lentiviral vector plasmid” means the plasmid bearing the genome of the lentiviral vector. In particular, the lentiviral vector is a HIV derived vector, especially a HIV-1 derived vector: in this case, the lentiviral vector plasmid comprises the cDNA of the nucleic acid sequences of the HIV that are necessary to enable replication of the vector genome recombined with heterologous sequence(s) such as the transgene encoding the inactive fluorescent protein comprised therein. Lentiviral vectors are well known from the person skilled in the art and their preparation based on HIV-1 virus has been disclosed in the art including in Zennou V. et al (Cell, 2000, 101, 173-185), Iglesias M.C. et al ( Mol. Ther, 2007, 15: 1203-1210). A lentivector plasmid based on HIV-1 genome in particular contains the HIV-1 cis-active elements (LTR (long terminal repeat), encapsidation signal Ψ, RRE and advantageously DNA Flap cPPT-CTS) operably linked with the transgene encoding the fluorescent protein under the control of a selected promoter, especially non-HIV promoter. In order to produce a lentiviral vector (i.e. vector particles or recombinant virus particles), the vector plasmid is transfected in cells suitable for assembly of the vector particles together with one or several further plasmids expressing HIV-1 genes necessary for the genome encapsidation, in particular one or a plurality of plasmids collectively comprising HIV-1 genes gag, pol, tat and rev and furthermore with an envelope expression plasmid encoding an envelope glycoprotein, in particular an envelope glycoprotein that does not originated from HIV and that may be obtained from the Vesicular Stomatitis Virus (VSV), e.g. a VSV-G protein. Specific description of the preparation of lentiviral plasmid vectors and complementation vectors based on HIV is disclosed in the art, in particular in patent applications WO.

According to a particular embodiment, the vector is a lentiviral vector plasmid selected from the group of: pLentiPuro_flipGFP_ER_3C (SEQ ID No.101), pLentiPuro_flipGFP_Membrane_3C (SEQ ID No.102), and pLentiPuro-flipGFP-3C (SEQ ID No.103). In another embodiment, the vector is a lentiviral vector plasmid selected from the group of: pLentiPuro-flipGFP-SARS2-HCVCore (SEQ ID No.150) and pLentiPuro-flipGFP-SARS2-HIVVpu (SEQ ID No.151).

In an alternative embodiment, the nucleotide sequence encoding the cleavage site of the enterovirus 3C in one of the above sequences of the vector is substituted for a nucleotide sequence encoding a cleavage site selected from the group of: ITTLGKFGQ (SEQ ID No.126) for the Enterovirus 2A protease, EALFQGPK (SEQ ID No.127) or SYFASEQGEIQWV (SEQ ID No.128) for the Enterovirus 3C protease, RAGAYIFS (SEQ ID No.129) for Alphaviruses, RELNGGAYTRYV (SEQ ID No.130), FTLKGGAPTKVT (SEQ ID No.131), IALKGGKIVNNW (SEQ ID No.132), TSAVLQSGFRKM (SEQ ID No.133), KVATVQSKMSDV (SEQ ID No.134), SAVKLQNNELSP (SEQ ID No.135), ATVRLQAGNATE (SEQ ID No.136), REPMLQSADAQS (SEQ ID No.137) and SGVTFQSAVKRT (SEQ ID No.138) for SARS-CoV-2 coronavirus, SGVTFQGKFKK (SEQ ID No.139) for SARS virus coronavirus, YAKRGGVF (SEQ ID No140) for Flaviviruses, and more particularly KERKRRGADTSI (SEQ ID No.141), TRSGKRSWPPSE (SEQ ID No.142), EPEKQRSPQDNQ (SEQ ID No.143), GLVKRRGGGTGE (SEQ ID No.144) for ZIKA viruses.

In an alternative embodiment, the nucleotide sequence encoding the cleavage site of the enterovirus 3C in one of the above sequences of the vector is substituted for a nucleotide sequence selected from the group of: GAGGACAGGGCCGGCGCCGGCATCATCGAGACCCCC for CHKV-1 (SEQ ID No.108) or GCCACCAGGGCCGGCTGCGCCCCCAGCTACAGGGTG for CHKV-2 (SEQ ID No.109) or CTGGACAGGGCCGGCGGCTACATCTTCAGCAGCGAC for CHKV-3 (SEQ ID No.110) or, ATCACCACTCTTGGGAAATTTGGACAA for EV71_2A (SEQ ID No.111) or AGCTACTTCGCCAGCGAGCAGGGCGAGATCCAGTGGGTG for EV71_3C (SEQ ID No.112) or, AGGGAGCTGAACGGCGGCGCCTACACCAGGTACGTG for SARSCoV2_1 (SEQ ID No.113) or TTCACCCTGAAGGGCGGCGCCCCCACCAAGGTGACC for SARSCoV2_2 (SEQ ID No.114) or ATCGCCCTGAAGGGCGGCAAGATCGTGAACAACTGG for SARSCoV2_3 (SEQ ID No.115) or ACCAGCGCCGTGCTGCAGAGCGGCTTCAGGAAGATG for SARSCoV2_4 (SEQ ID No.116) or AGCGGCGTGACCTTCCAGAGCGCCGTGAAGAGGACC for SARSCoV2_5 (SEQ ID No.117) or AAGGTGGCCACCGTGCAGAGCAAGATGAGCGACGTG for SARSCoV2_6 (SEQ ID No.118) or AGCGCCGTGAAGCTGCAGAACAACGAGCTGAGCCCC for SARSCoV2_7 (SEQ ID No.119) or GCCACCGTGAGGCTGCAGGCCGGCAACGCCACCGAG for SARSCoV2_8 (SEQ ID No.120) or AGGGAGCCCATGCTGCAGAGCGCCGACGCCCAGAGC for SARSCoV2_9 (SEQ ID No.121) or, AAGGAGAGGAAGAGGAGGGGCGCCGACACCAGCATC for ZIKV_1 (SEQ ID No.122) or ACCAGGAGCGGCAAGAGGAGCTGGCCCCCCAGCGAG for ZIKV_2 (SEQ ID No.123) or GAGCCCGAGAAGCAGAGGAGCCCCCAGGACAACCAG for ZIKV_3 (SEQ ID No.124) or GGCCTGGTGAAGAGGAGGGGCGGCGGCACCGGCGAG for ZIKV_4 (SEQ ID No.125).

The vector is used for the transformation of host cells for the expression of the inactive form of the fluorescent reporter protein. When the vector is a lentiviral vector plasmid, it is used for the preparation of recombinant lentiviral particles in a host cell having recourse to complementation plasmids bearing the nucleotide sequences for the expression of necessary structural proteins according to methods known in the art. The vector may be designed for transient expression of the nucleic acid construct of the invention of for stable expression of said nucleic acid construct. The lentiviral vector particles may in particular be advantageously used for the stable expression of the nucleic acid construct of the invention.

The invention also relates to a cell which is a prokaryotic or a eukaryotic cell or cell line transformed with the recombinant nucleic acid according to the invention or with a transformation vector. In a specific embodiment, the cell is selected for its sensibility to infection by a determined human virus targeting the cleavage site of the inactive fluorescent protein expressed in the cell, and said cell is optionally a stable cell line. A stable cell line may advantageously stably express the nucleic acid construct of the invention. It may be obtained after transduction with recombinant lentiviral vector particles encoding the nucleic acid construct of the invention wherein said construct provides the sequence coding for the recombinant inactive fluorescent reporter protein to the nucleus of the host cells and allow its insertion in its genome. According to such embodiment, the invention concerns a cell line stably expressing the nucleic acid construct as defined herein, wherein the nucleic acid construct is inserted in the genome of the cell.

The invention also relates to recombinant viral vector particles, in particular HIV-1 vector particles which comprise as their genome a nucleic acid construct as defined herein. Such recombinant viral particles may be used for transduction of cells in a biological sample assayed for the detection of a virus wherein the targeted virus is one recognizing the cleavage site introduced in the inactive fluorescent reporter protein. Alternatively, the recombinant viral particles may be used for transduction of host cells that are used to assay the biological sample in which detection of viral infection is performed and wherein the targeted virus is one recognizing the cleavage site introduced in the inactive fluorescent reporter protein.

The invention is also directed to a recombinant fluorescent reporter protein which is encoded by a nucleic acid construct or a recombinant nucleic acid as disclosed herein.

A particular inactive recombinant fluorescent reporter protein according to the invention is characterized by its amino acid sequence which is the sequence encoded by any polynucleotide selected from the group of SEQ ID No.47 to SEQ ID No.100.

The invention also concerns a polynucleotide encoding a viral protease cleavage site which is selected from the group of: GAGGACAGGGCCGGCGCCGGCATCATCGAGACCCCC for CHKV-1 (SEQ ID No.108) or GCCACCAGGGCCGGCTGCGCCCCCAGCTACAGGGTG for CHKV-2 (SEQ ID No.109) or CTGGACAGGGCCGGCGGCTACATCTTCAGCAGCGAC for CHKV-3 (SEQ ID No.110) or, ATCACCACTCTTGGGAAATTTGGACAA for EV71_2A (SEQ ID No.111) or AGCTACTTCGCCAGCGAGCAGGGCGAGATCCAGTGGGTG for EV71_3C (SEQ ID No.112) or, AGGGAGCTGAACGGCGGCGCCTACACCAGGTACGTG for SARSCoV2_1 (SEQ ID No.113) or TTCACCCTGAAGGGCGGCGCCCCCACCAAGGTGACC for SARSCoV2_2 (SEQ ID No.114) or ATCGCCCTGAAGGGCGGCAAGATCGTGAACAACTGG for SARSCoV2_3 (SEQ ID No.115) or ACCAGCGCCGTGCTGCAGAGCGGCTTCAGGAAGATG for SARSCoV2_4 (SEQ ID No.116) or AGCGGCGTGACCTTCCAGAGCGCCGTGAAGAGGACC for SARSCoV2_5 (SEQ ID No.117) or AAGGTGGCCACCGTGCAGAGCAAGATGAGCGACGTG for SARSCoV2_6 (SEQ ID No.118) or AGCGCCGTGAAGCTGCAGAACAACGAGCTGAGCCCC for SARSCoV2_7 (SEQ ID No.119) or GCCACCGTGAGGCTGCAGGCCGGCAACGCCACCGAG for SARSCoV2_8 (SEQ ID No.120) or AGGGAGCCCATGCTGCAGAGCGCCGACGCCCAGAGC for SARSCoV2_9 (SEQ ID No.121) or, AAGGAGAGGAAGAGGAGGGGCGCCGACACCAGCATC for ZIKV_1 (SEQ ID No.122) or ACCAGGAGCGGCAAGAGGAGCTGGCCCCCCAGCGAG for ZIKV_2 (SEQ ID No.123) or GAGCCCGAGAAGCAGAGGAGCCCCCAGGACAACCAG for ZIKV_3 (SEQ ID No.124) or GGCCTGGTGAAGAGGAGGGGCGGCGGCACCGGCGAG for ZIKV_4 (SEQ ID No.125).

The invention also relates to a polypeptide which is a cleavage site for a virus protease and which is selected from the group of SEQ ID No. 126 to SEQ No.144.

The invention also contemplates the use of a nucleic acid construct or of a vector or a protein disclosed herein, for in vitro detection of a viral infection in a biological sample of a human or animal subject, wherein the detection targets a virus expressing a protease recognizing the cleavage site inserted in the recombinant inactive fluorescent reporter protein.

According to a particular embodiment, the invention relates to the use of a cell line as disclosed herein for in vitro detection and optionally quantification of a viral infection in a biological sample of a human or animal subject, wherein the detection targets a virus recognizing the cleavage site contained, in particular recombined, in the inactive fluorescent reporter protein. According to a particular embodiment, fluorescence of the reporter protein is detected or measured directly on the cells.

The invention also relates to an in vitro method of detecting or monitoring a pathogen infection, in particular a virus infection, in a biological sample previously obtained from a human or an animal subject, which comprises the steps of:
a. Providing cells according to the invention that express either transiently or stably an inactive fluorescent reporter protein comprising a cleavage site for a protease of the virus to be detected,
b. Contacting said cells with the assayed biological sample in conditions that enable the virus when present in the sample, to infect the cells and its protease to cleave the protease cleavage site,
c. Allowing fluorescence to increase in the cells that have been contacted with the biological sample following activation of the inactive fluorescent reporter protein by cleavage in step b. of the viral protease cleavage site and measuring said fluorescence of the reporter protein,
d. Optionally comparing the fluorescence level of the active fluorescent reporter protein to a standard or fluorescent control protein expressed in the cells and optionally concluding on virus infectious activity in the subject and/or quantitating the virus.

Alternatively, an in vitro method of detecting or monitoring a pathogen infection, in particular a virus infection, in a biological sample previously obtained from a human or an animal subject, according to the invention, comprises the steps of:
a. Providing an inactive fluorescent protein as a reporter protein expressed from the nucleic acid construct of any one of the disclosed embodiments, together with a control protein expressed from the same nucleic acid construct wherein the inactive fluorescent reporter protein comprises a cleavage site for a protease of the virus to be detected,
b. Contacting said inactive fluorescent reporter protein with the assayed biological sample in conditions that enable the virus protease to target and to cleave the protease cleavage site in the inactive fluorescent reporter protein,
c. Allowing fluorescence to increase in the biological sample following activation of the inactive fluorescent reporter protein by cleavage in step b. and measuring said fluorescence,
d. Optionally comparing the fluorescence level to a standard or to the fluorescence of the control protein and optionally concluding on the virus infectious activity in the subject and/or quantitating the virus.

These methods may be used for the diagnosis of a human subject or an animal for an infection by a determined pathogen, especially a determined virus, including at an early stage of infection when the pathogen load is still low and its proteases are however expressed sufficiently to enable the fluorescence of the assayed sample to increase over the background fluorescence of the sample, as a result of the method carried out.

The methods may also be used for monitoring the infection by a pathogen, in particular a virus in a subject that is undergoing therapy for this infection.

A particular embodiment of the method of detecting or monitoring a virus infection in a biological sample previously obtained from a human or an animal subject, is targets infection by a virus selected in the group of Alphaviruses, Coronaviruses, Enteroviruses, Retroviruses and Flaviviruses, in particular a virus which is a coronavirus, especially is SARS-CoV-2 (or SARS-2) responsible for Covid-19.

In a particular embodiment, the methods of detecting a pathogen infection, in particular a virus infection, in a biological sample previously obtained from a human or an animal subject, may be carried out as soon as 24 hours, in particular as soon as 14 hours, more particularly within a range of 8 to 24 hours, following suspicion of infection.

Accordingly, the invention enables performing an early detection of a pathogen infection, in particular a virus infection, such as infection by SARS-CoV-2 virus as soon as 24 hours following suspicion of infection or even in a range of 8 to 24 hours or 8 to 14 hours following suspicion of infection.

Furthermore, the invention concerns a laboratory animal for experimental or clinical observation of the response to a pathogen infection, in particular a virus infection, wherein the animal has been transformed to enable its genome to express a nucleic acid construct or a recombinant nucleic acid, either transiently or stably, the animal being in particular a rodent, an insect or a non-human mammal.

The invention will be further described in the examples and figures that follow that illustrate particular non limitative embodiments.

### LEGEND OF THE FIGURES

FIG. 1: sequence alignment for GFP

FIG. 2: nucleotide sequence and encoded amino acid sequence for pLentiPuro_flipGFP_3C

FIG. 3: nucleotide sequence and encoded amino acid sequence for pLentiPuro_flipGFP_ER_3C

FIG. 4: nucleotide sequence and encoded amino acid sequence for pLentiPuro_flipGFP_Membrane_3C

FIGS. 5 to 7: flipGFP_Schematics

FIG. 8: flipGFP by Transfection and Transfected Protease - flipGFP detection of SARS CoV-2 protease:

HEK293T cells were seeded in 24 well plates. Cells were transfected with the SARS-CoV-2 cleavage-site 3 ER Retention signal peptide flipGFP construct alone (Mock) or together with a plasmid expressing the Nsp5 protease (+Nsp5). Cells were incubated for 24 hours, before fixing with 4% Formalin. Cells were imaged using an EVOS FL microscope.

FIG. 9: flipGFP by Transfection and Virus Infection - flipGFP detection of chikungunya virus infection, enterovirus 71 infection, CHKV virus infection, SARS CoV-2 infection, ZIK virus infection: Vero E6 cells were seeded in 24 well plates. Cells were transfected with the individual flipGFP constructs. After 24 hours cells were infected with either enterovirus 71 (EV71), chikungunya virus (CHKV), SARS-CoV-2 (BetaCoV/France/IDF0372/2020, SARS-CoV-2) or Zika virus (ZIKV) at an MOI = 1 (SARS2). Cells were incubated for a further 48 hours, before fixing with 4% Formalin. Cells were imaged using an EVOS FL microscope.

FIG. 10: flipGFP expressed in Stable Cell Line and Infected- Stable cell line FlipGFP detection of SARS Cov-2 infection - A549 cells overexpressing Ace2 were transduced using a lentivirus encoding the SARS-CoV-2 flipGFP Membrane 3 construct. Cells were selected using puromycin, followed by flow-cytometry sorting, gating for high mCherry and GFP negative expressing cells. Cells were seeded in 24 well plates. Cells were infected with SARS-CoV-2 (BetaCoV/France/IDF0372/2020) at an MOI = 1 (SARS2) or left uninfected (Mock). Cells were incubated for 48 hours, before fixing with 4% Formalin. Cells were imaged using an EVOS FL microscope.

FIG. 11: A: Map of lentiviral plasmid pLentiPuro-flipGFP-SARS2-HCVCore (encompassing the sequence of flipGFP-SARS2-HCVCore) B: detail of the transgene C: nucleotide sequence of the insert of flipGFP-SARS2-HCVCore: SEQ ID No.150. In this figure the segment designated as flipGFP (SEQ ID No. 149) refers to the subdomains of GFP that have been switched (flipped) from their position in the native GFP and also contain the sequence of the protease cleavage site. More accurately flipGFP is featured by this segment together with the GFP1-9 segment (SEQ ID No. 148).

FIG. 12: A: Map of lentiviral plasmid pLentiPuro-flipGFP-SARS2-HIVVpu (encompassing the sequence of flipGFP-SARS2-HIVVpu) B: detail of the transgene C: nucleotide sequence of the insert of flipGFP-SARS2-HIVVpu: SEQ ID No.151. In this figure the segment designated as flipGFP refers to the subdomains of GFP that have been switched (flipped) from their position in the native GFP and also contain the sequence of the protease cleavage site (SEQ ID No. 149). More accurately flipGFP is featured by this segment together with the GFP1-9 segment (SEQ ID No. 148).

FIG. 13: VeroE6 cells were transfected with flipGFP-SARS2-10-HCV plasmids. After 24 hours cells were trypsinised and seeded on µ-slides (ibidi). After further 24 hours cells were either Mock infected or infected with SARS-CoV-2 for 1 hour, before replacing the media. The samples were incubated and imaged in a Nikon Biostation with 5% CO₂ at 37° C. Images were taken every 20 minutes over 48 hours using 20x magnification and 488 nm (GFP), as well as phase contrast channels. Subsequently still images were isolated in ImageJ software at the indicated times. Contrast of the images were inverted. Scale bars (black) represent 50 µm.

FIG. 14: VeroE6 cells transfected with flipGFP-SARS2-10-HCV were infected with SARS-CoV-2 and imaged under life-cell conditions every 20 minutes for 48 hours using a Nikon Biostation at 20x magnification. Images from the GFP channel were processed in ImageJ and the signal was inverted. The earliest detection of a GFP signal from SARS-CoV-2 infected cells was at around 9.6 hours post infection. The development of the reporter cell signal was stable and increased over time. Times of GFP channel images are shown. White arrows indicate cells with GFP signal. Black scale bars represent 50 µm.

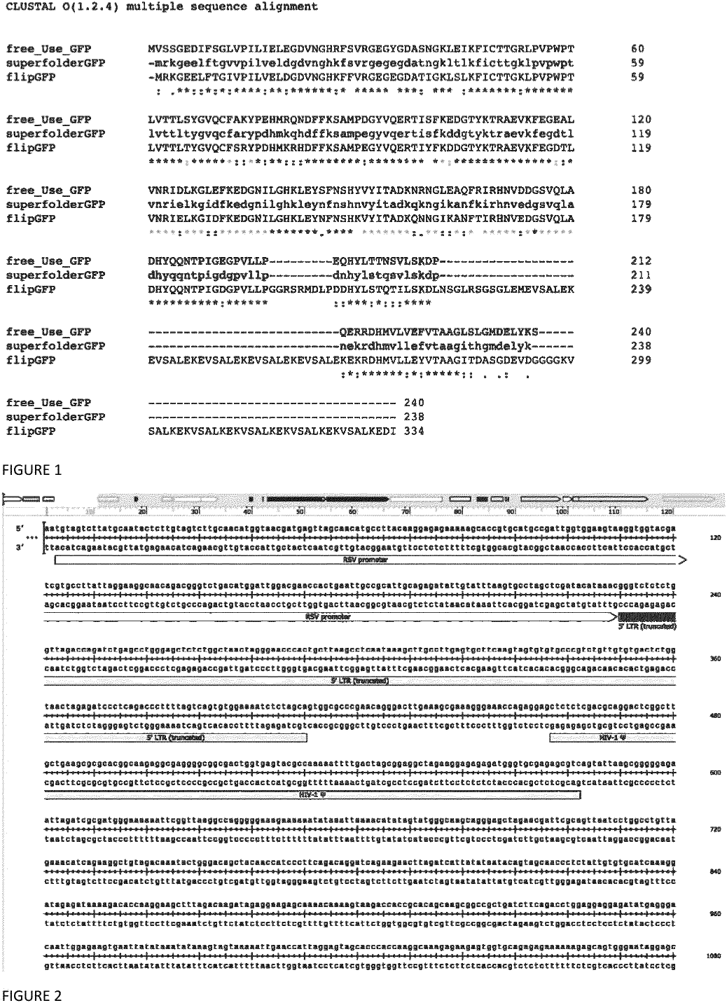

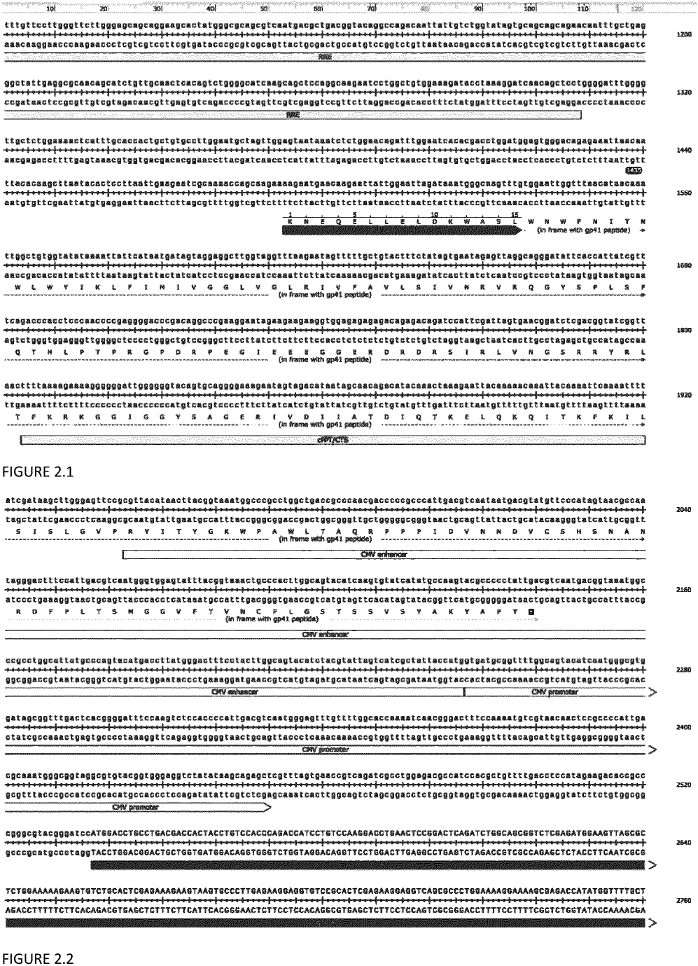

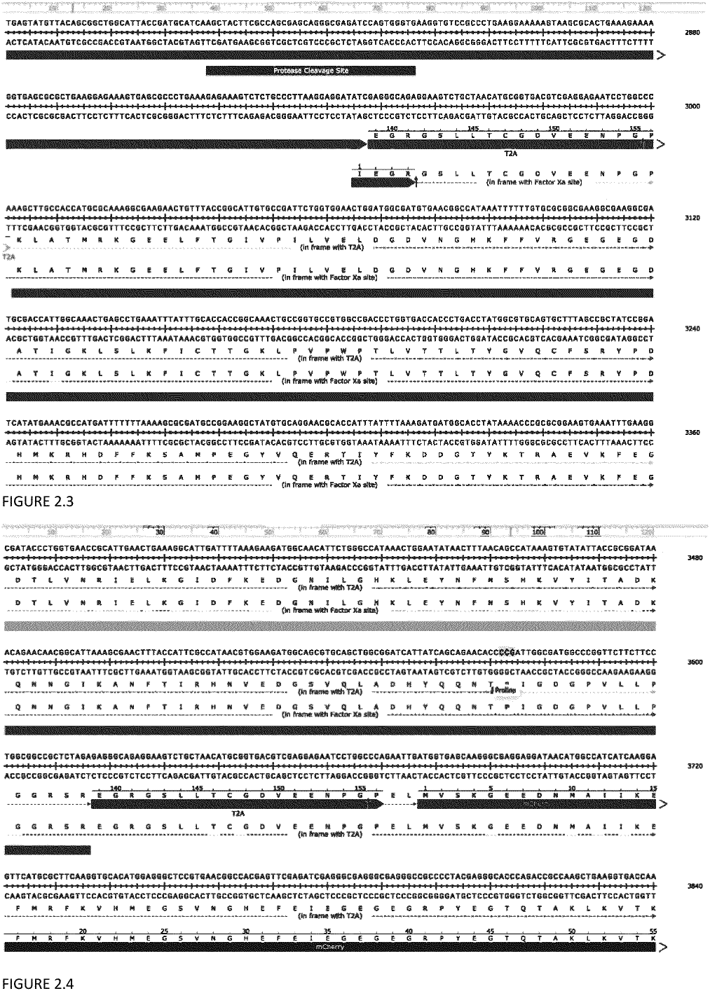

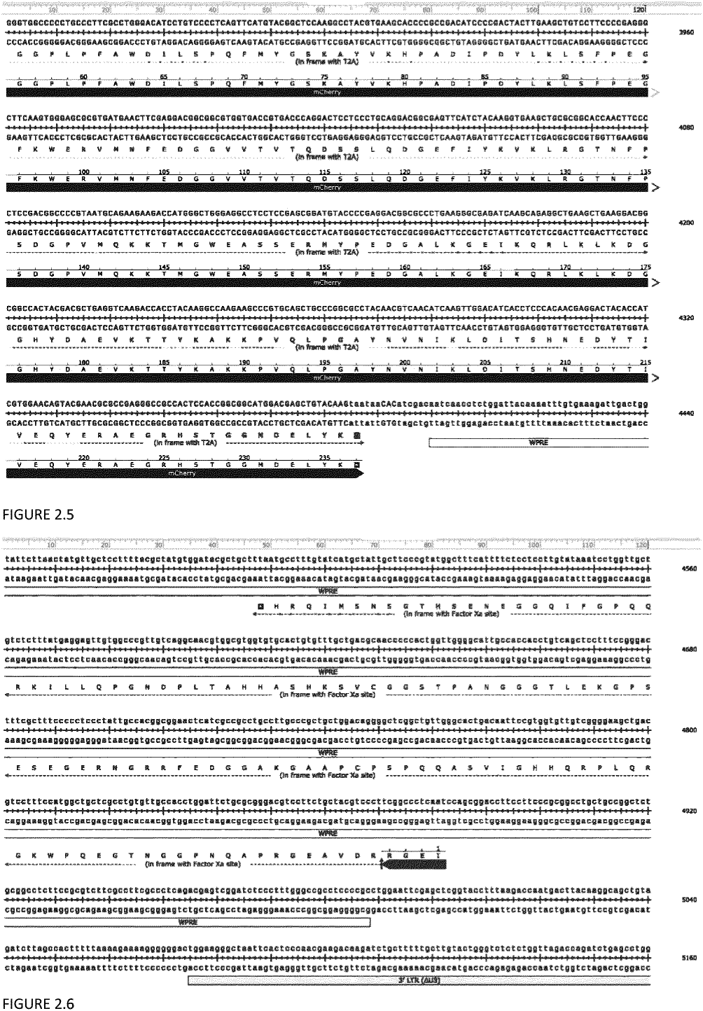

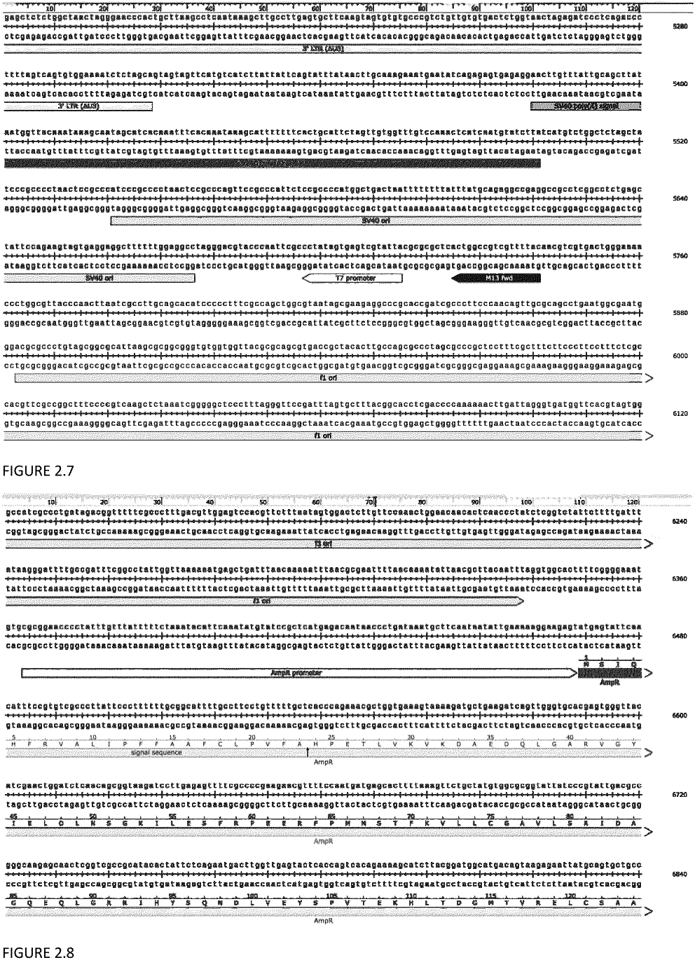

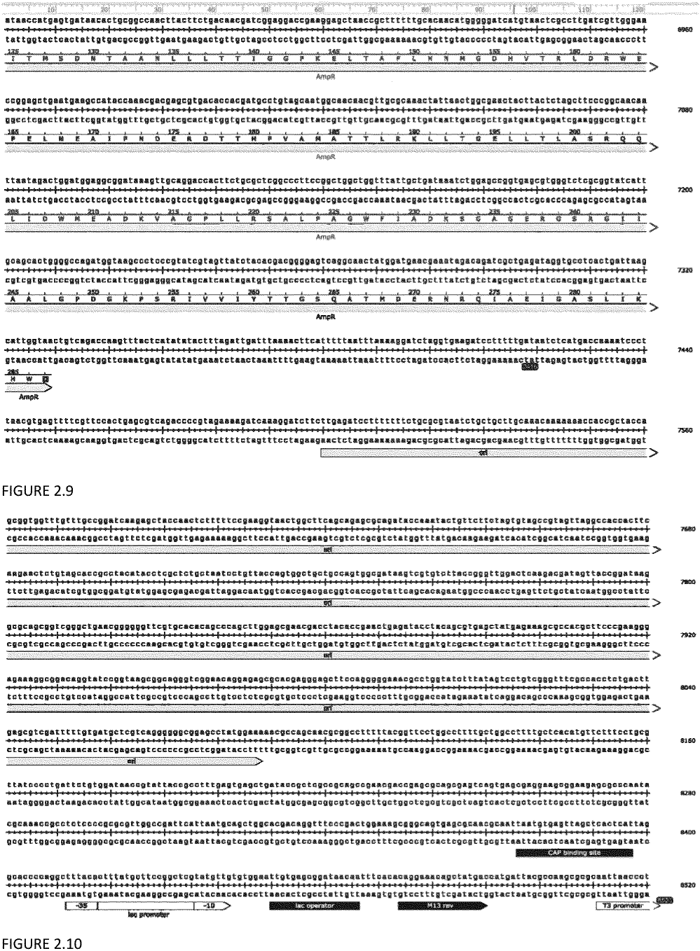

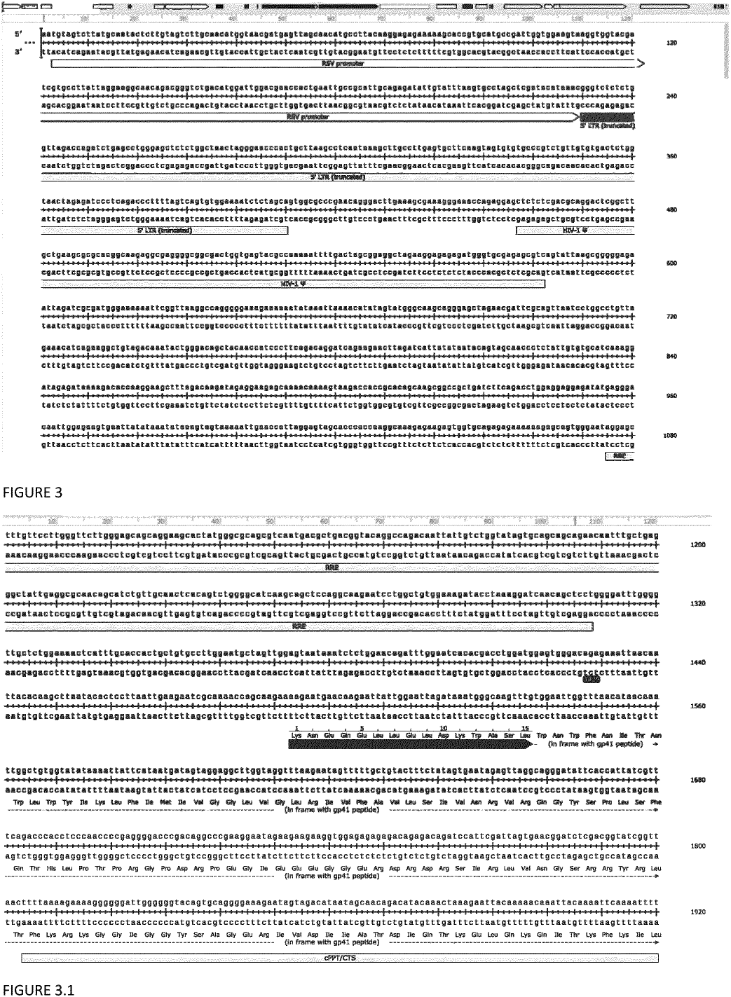

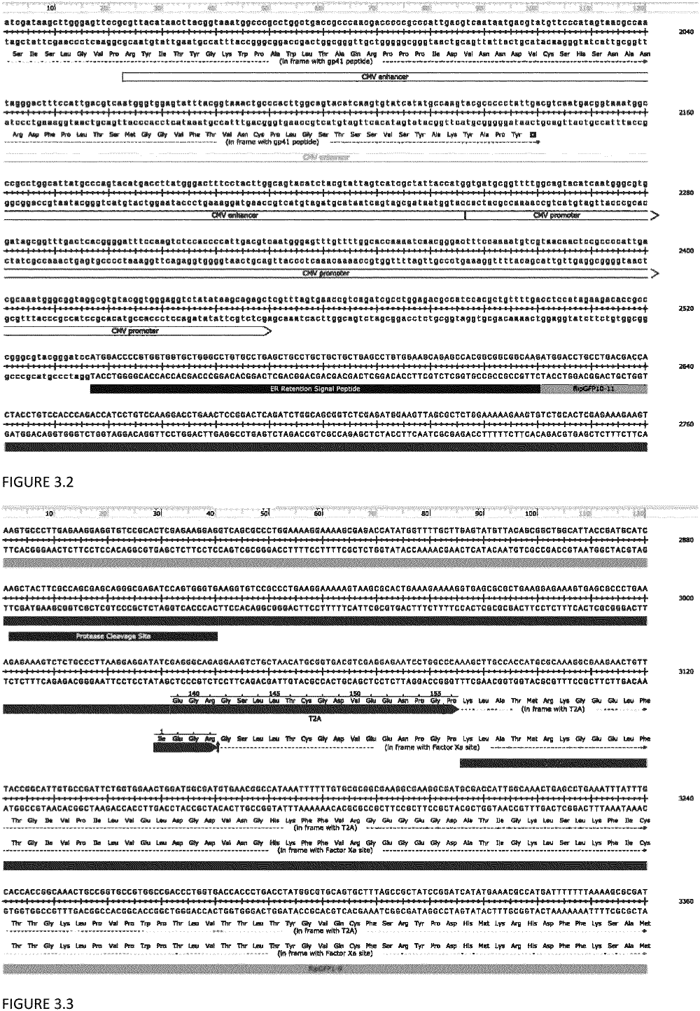

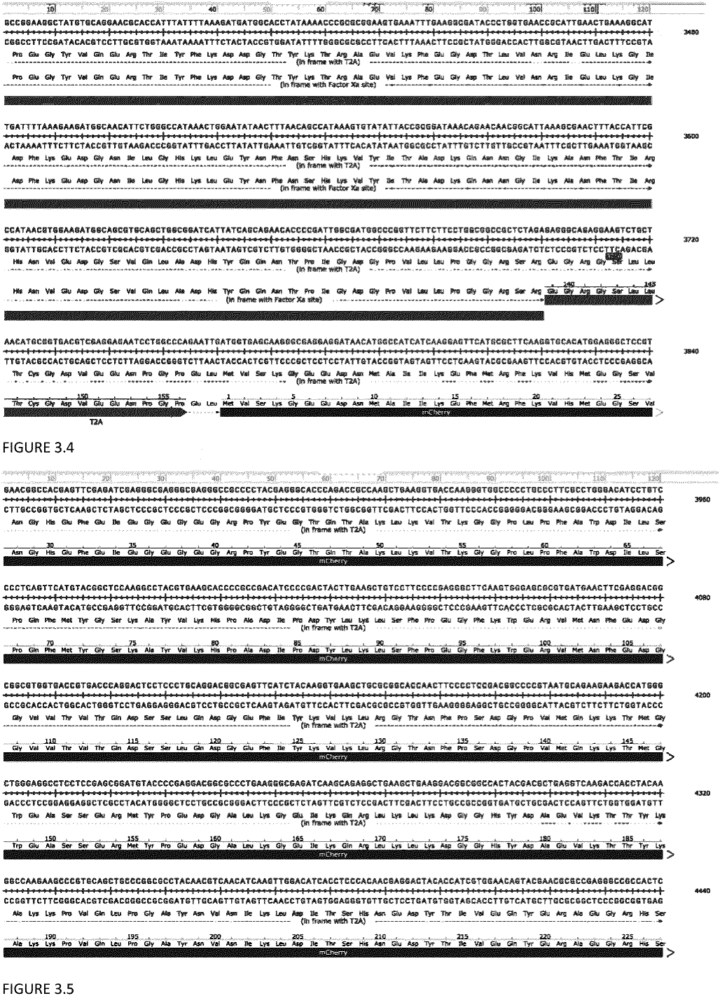

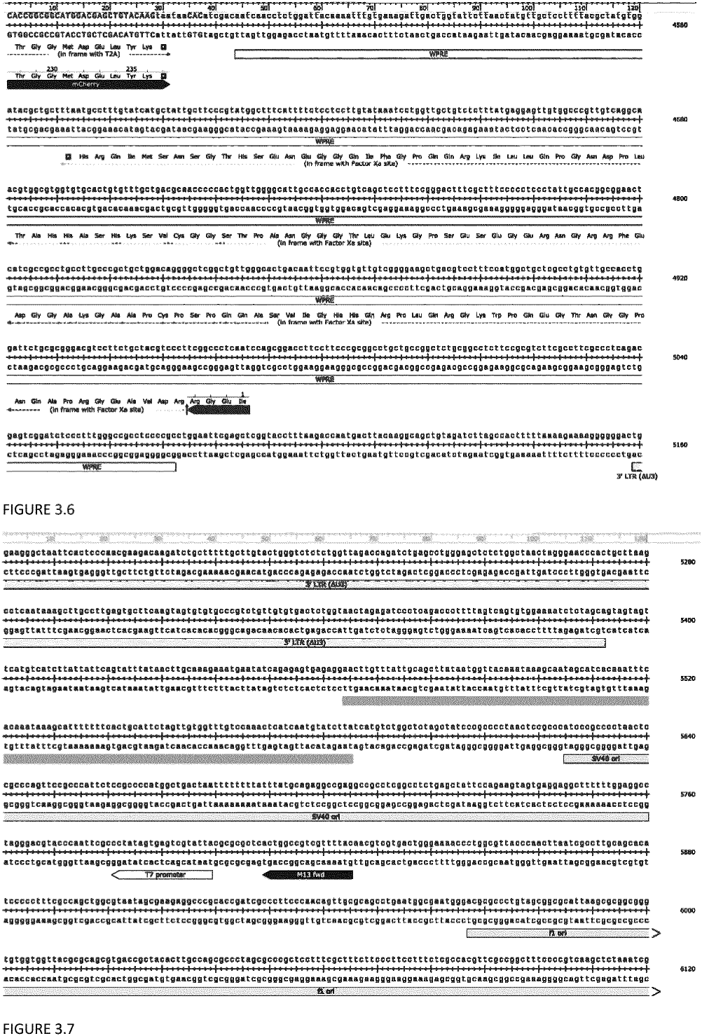

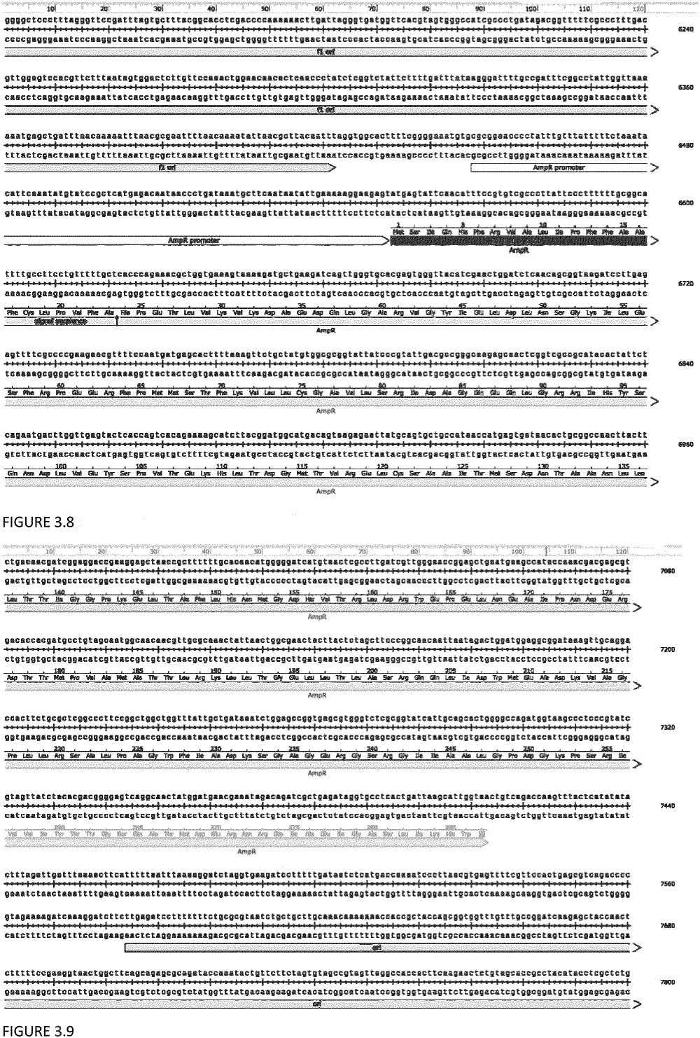

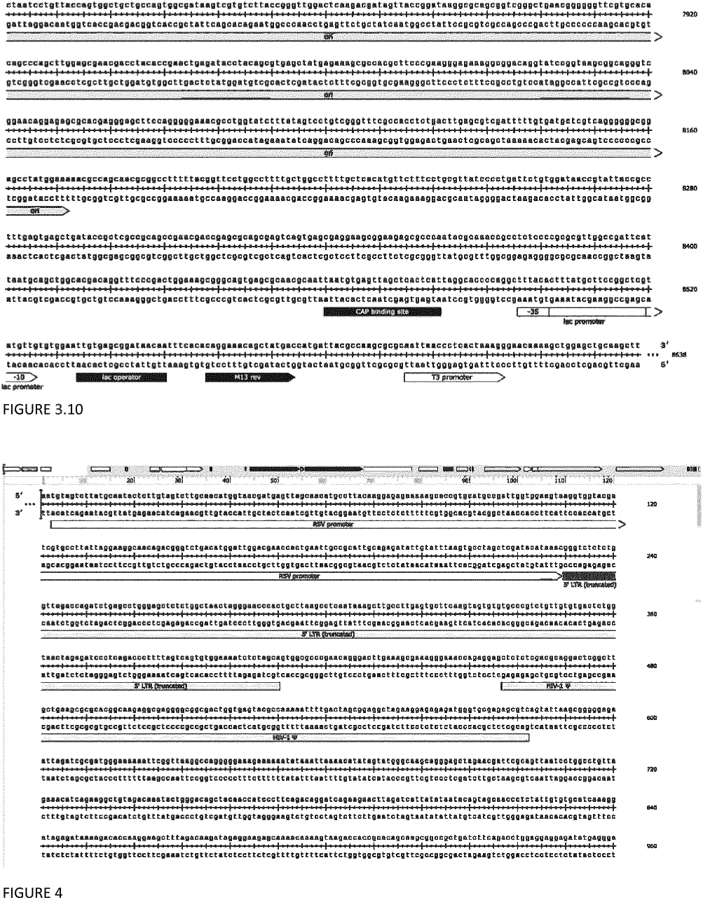

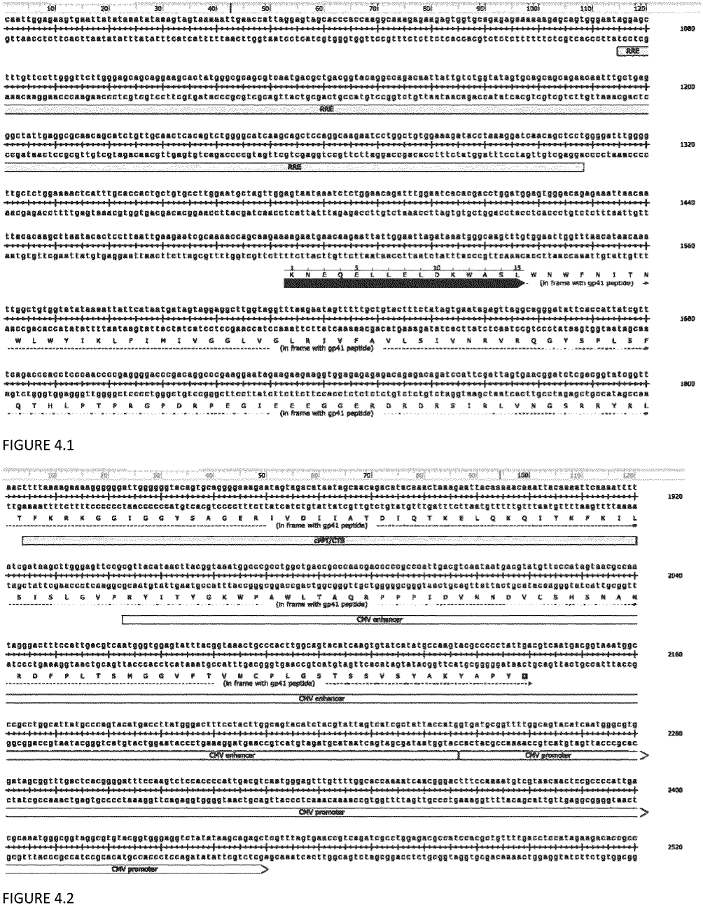

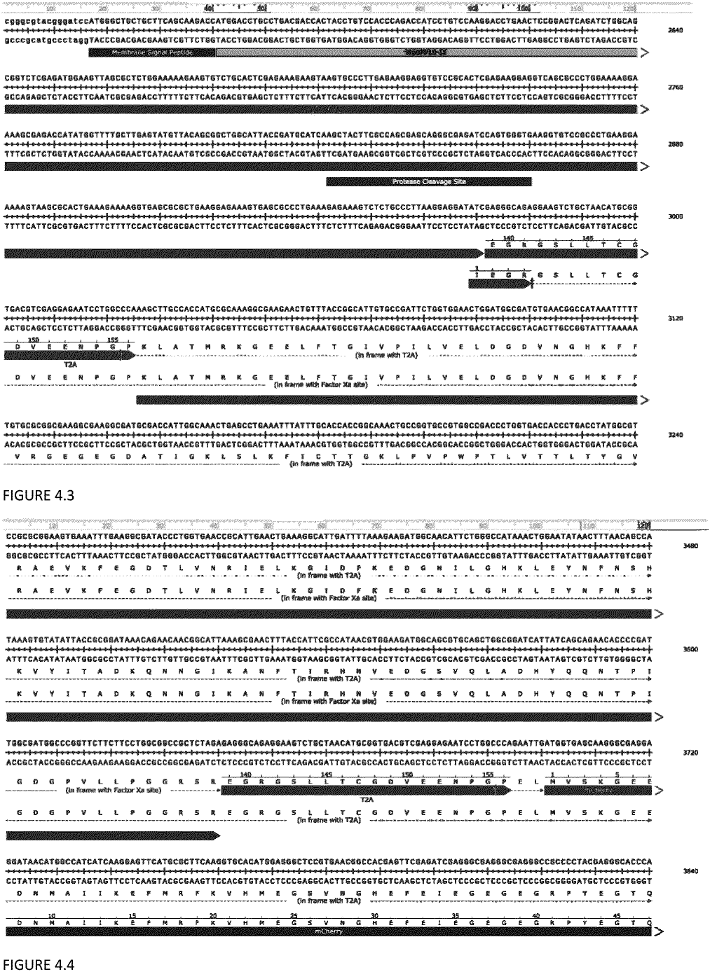

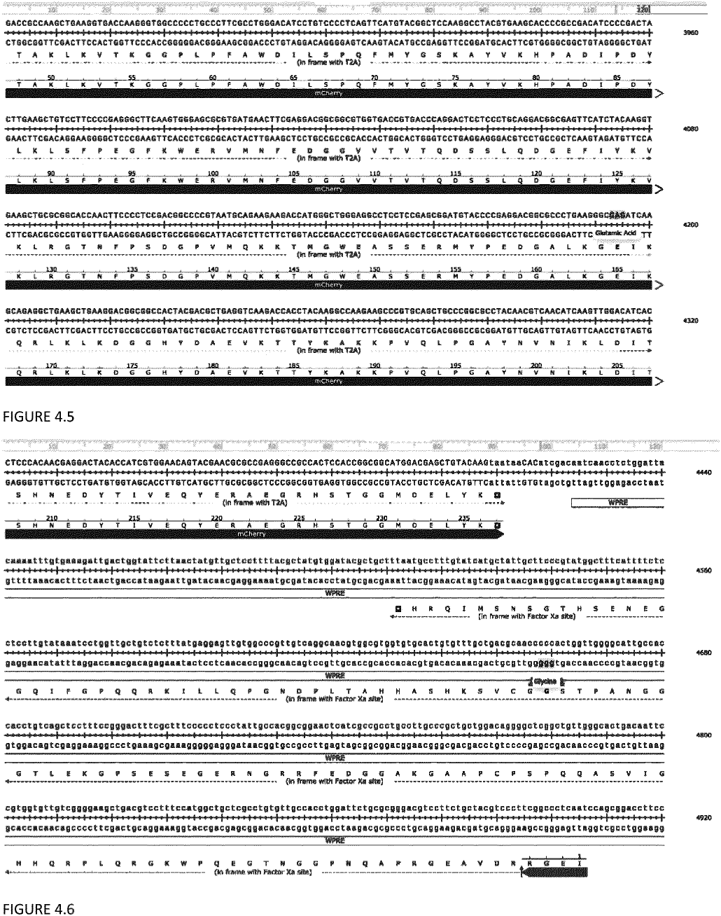

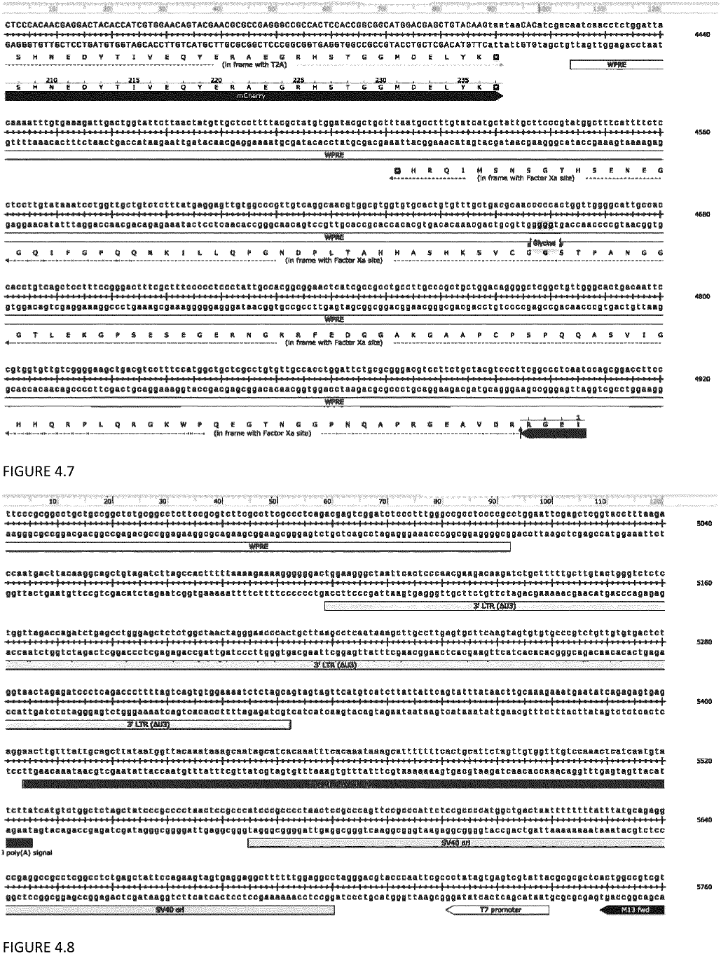

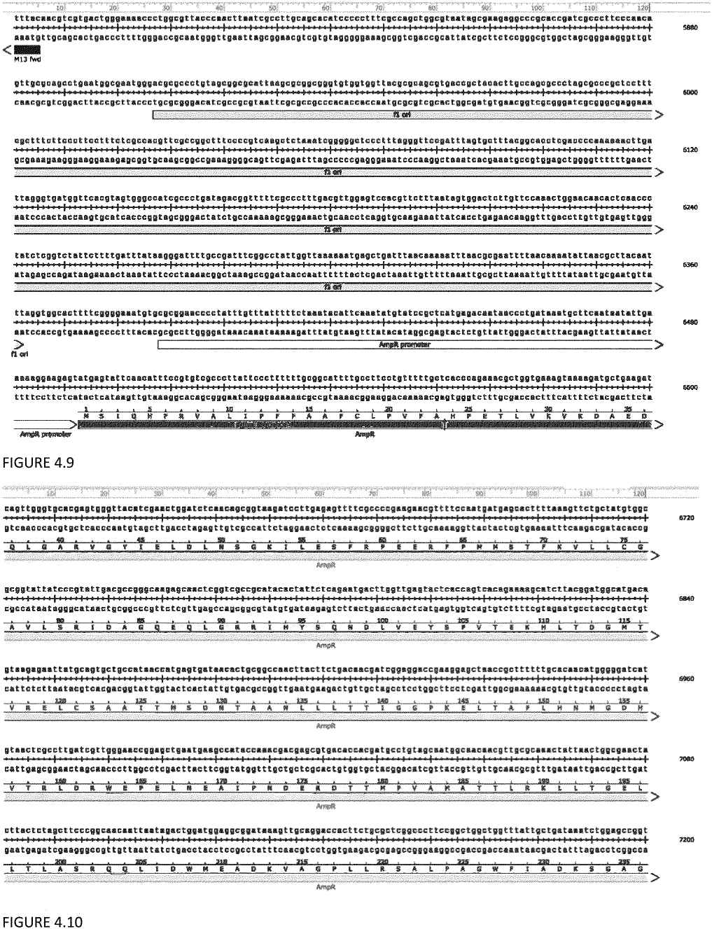

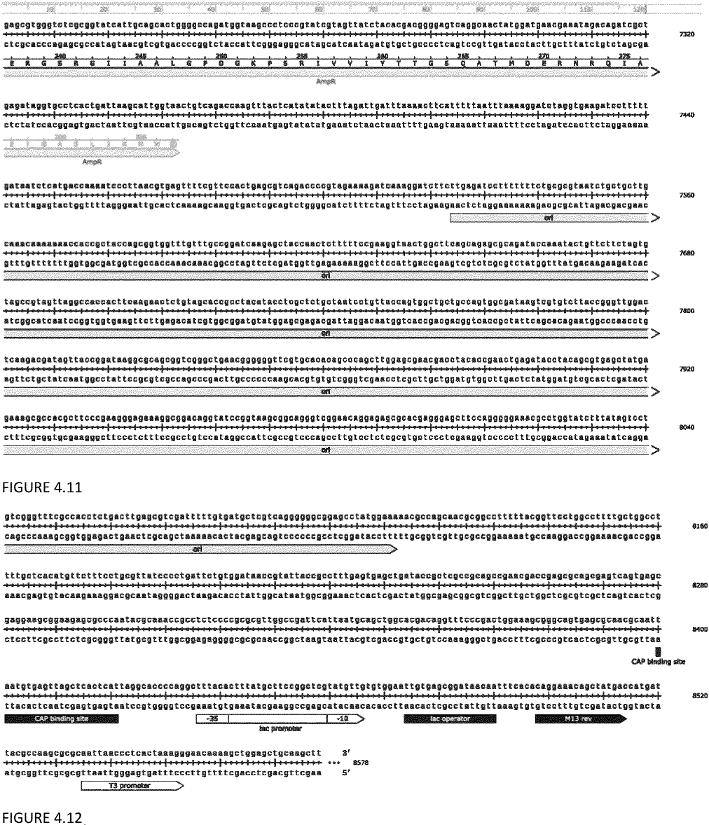

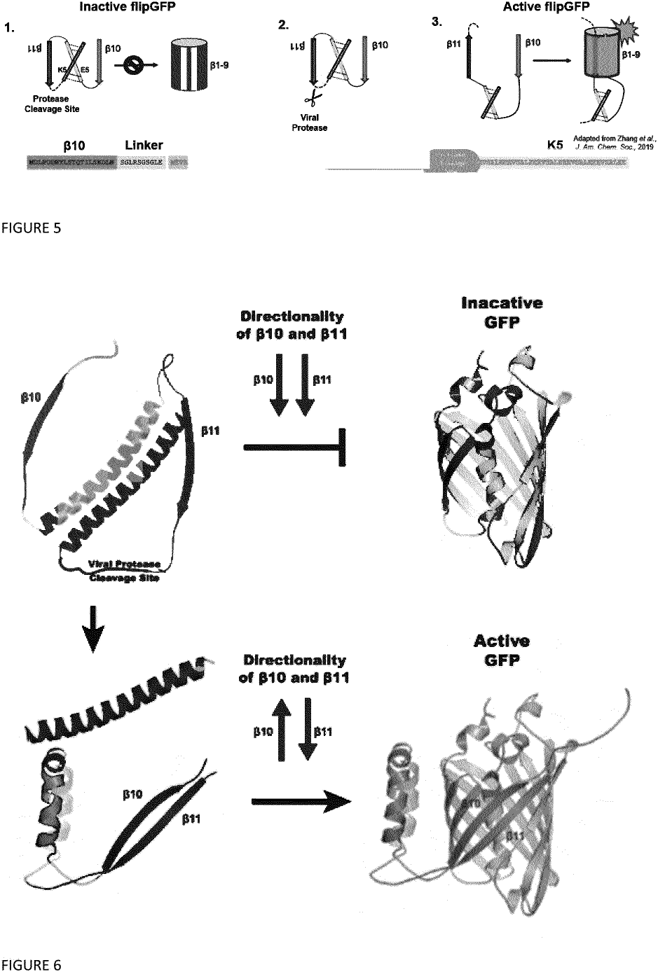

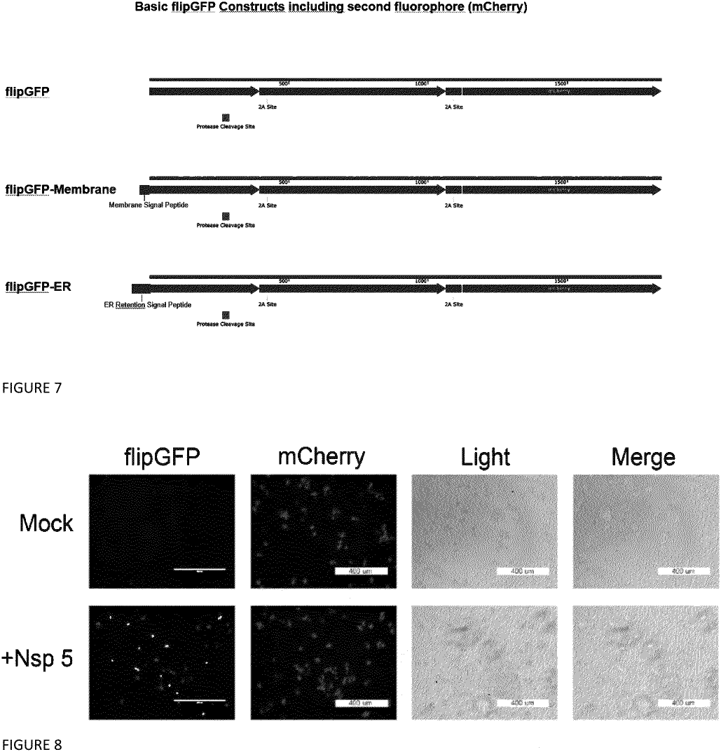

### EXPERIMENTAL DATA

### Methods

### Cells and Viruses

Vero E6 (Vero 76, clone E6, Vero E6, ATCC® CRL-1586TM) were maintained in a humidified atmosphere at 37° C. with 5% CO₂, in Dulbecco’s modified Eagle’s medium (DMEM, Life Technologies) containing 10% (v/v) fetal bovine serum (FBS, Life Technologies). ACE2-expressing A549 cells, a human lung epithelial cell line (Institut Pasteur; Bouhaddou M. et al - The Global Phosphorylation landscape of SARS-CoV-2 infection https://doi.org/10.1016/j.cell.2020.06.034). A549-ACE2 cells were cultured in DMEM supplemented with 10% (v/v) FBS and maintained at 37° C. with 5% CO₂. HEK293T cells were DMEM supplemented with 10% (v/v) FBS and maintained at 37° C. with 5% CO₂.

SARS-CoV-2 (BetaCoV/France/IDF0372/2020 isolate) was supplied through the European Virus Archive goes Global (EVAg) platform. The virus was propagated in Vero E6 cells in DMEM supplemented with 2% FBS.

Enterovirus 71 (EV71) Sep006 isolate (GenBank: KX197462.1) was generated using plasmid-based reverse genetics system. For this, the virus sequence was synthesized and cloned into a pCAGGS plasmid that flanked the virus genome sequence with a hammer-head ribozyme (HHRz) at the 5′ end and a hepatitis delta ribozyme (HdRz) sequence at the 3′ end.

Zika virus used for the identification of DVGs is the African strain MR766 of Zika virus (ZIKV). Accession number for this strain is Genbank # LC002520.1.

Chikungunya virus was generated from CHIKV infectious clones derived from the Indian Ocean lineage, ECSA genotype. Accession number for such strain SZ 1050 of this lineage is Genbank # MG664850.1.

All experiments with live viruses were performed in compliance with Institut Pasteur Paris’s guidelines for Biosafety Level 3 (BSL-3) containment procedures in approved laboratories. All experiments were performed in at least three biologically independent samples.

### flipGFP Plasmids

The template for the flipGFP plasmids was kindly provided by Xiaokun Shu (University of California, San Francisco) (Zhang et al., 2019). The lentivial plasmid pLenti-puro (Guan et al., 2011) was acquired through Addgene (#39481). The plasmid and flipGFP insert were linearized by PCR (see Table 1 for primer sequence). The two fragments were joined using InFusion cloning (Clonetech) according to the manufacturer’s instructions. The plasmid was confirmed by Sanger sequencing. The nucleotide sequences for the individual protease cleavage sites were changed by single PCR reactions (Q5, Thermo Scientific) (see Table 1 for primers). The correctness of the inserted sequences was confirmed by Sanger sequencing.

The membrane signal peptide was inserted by a single PCR reaction (Q5, Thermo Scientific) (see Table 1 for primers) before changing the nucleotide sequences for the individual protease cleavage sites by PCR.

The endoplasmatic reticulum (ER) retention signal peptide was inserted by linearizing the pLenti-puro-flipGFP plasmid (see Table 1 for primers) and inserting the nucleotide sequence of the ER retention signal peptide using annealed oligonucleotides by InFusion cloning (Clonetech) according to the manufacturer’s instructions. The correct insertion of the peptide sequence was confirmed by Sanger sequencing. The individual protease cleavage sites were altered by PCR (Q5, Thermo Scientific).

### Transient flipGFP Expression

HEK293T or Vero E6 cells were seeded on cover slips. The following day cells were transfected with individual flipGFP plasmids using TransIT-LT1 (Mirus) according to the manufacturer’s instructions. For infections, transfected cells were incubated for 24 hours prior to infections. To assess the activity of individual SARS-CoV-2 flipGFP constructs, flipGFP plasmids were co-transfected with either pLVX-EF1alpha-nCoV2019-nsp5-2xStrep-IRES-Puro or pLVX-EF1alpha-nCoV2019-nsp5-C145A-2xStrep-IRES-Puro (kindly provided by Nevan Krogan, University of California, San Francisco, Gordon et al., 2020) for the expression of the viral protease.

### Stable flipGFP Expression Cells

Individual flipGFP constructs in pLenti-puro were transfected together with pAX2 (Proc Natl Acad Sci U S A. 2011 Jun 21;108(25):10343-8. Epub 2011 Jun 6. This plasmid is available through Addgene) and pCMV-VSV-G (published in RNA 2003 Apr;9(4):493-501. This plasmid is available through Addgene) in HEK293T. Lentiviruses were harvested 48 hours post transfection and supernatants were cleared from cellular debris by centrifugation. A549-Ace2 cells were seeded and individual lentivirus containing the flipGFP constructs were used to transduce the target cells using 6 µg/ml Diethylaminoethyl (DEAE)-dextran (Sigma) together with centrifugation using 1,000x g for 1 hour. After two days of incubation, cells containing flipGFP were selected using 10 µg/ml puromycin (Sigma). To obtain a homogeneous population of flipGFP expressing cells, cells were FACS-sorted using mCherry expression as marker for the presence and expression of flipGFP.

### Detection of Viral Protease and Virus Infection in Transfected Cells and in Stable Cells

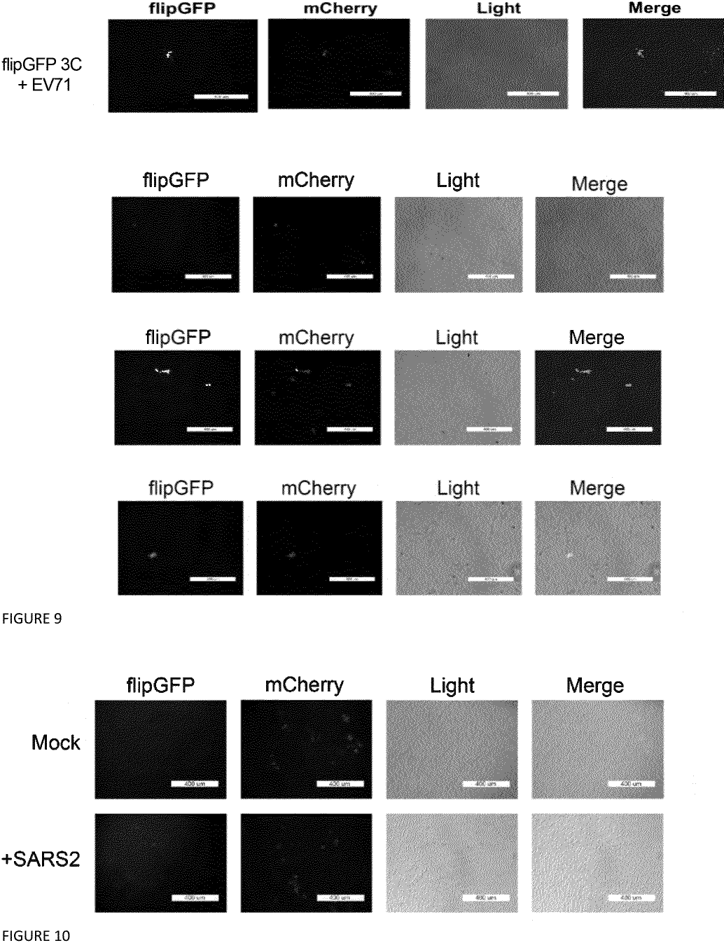

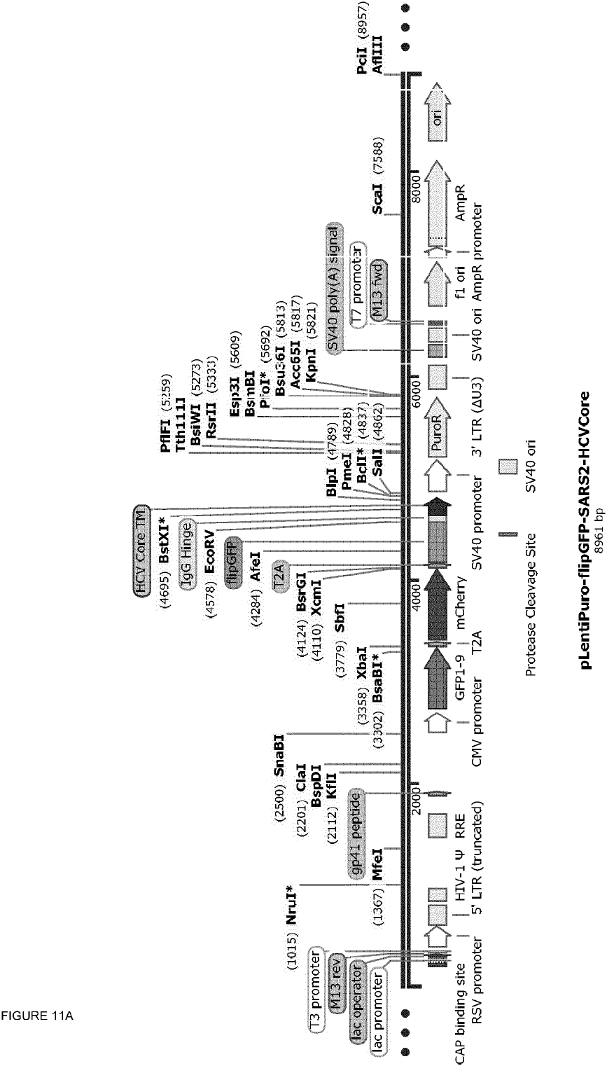

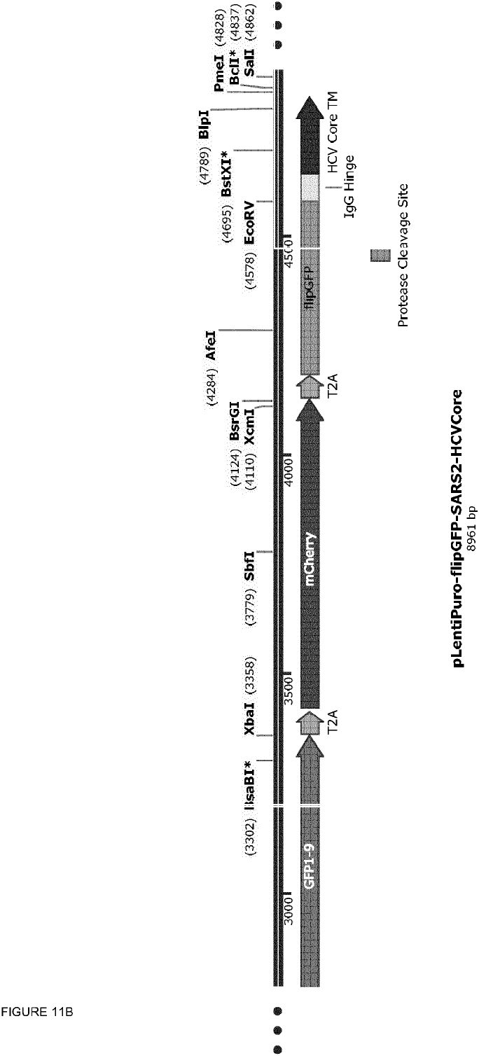

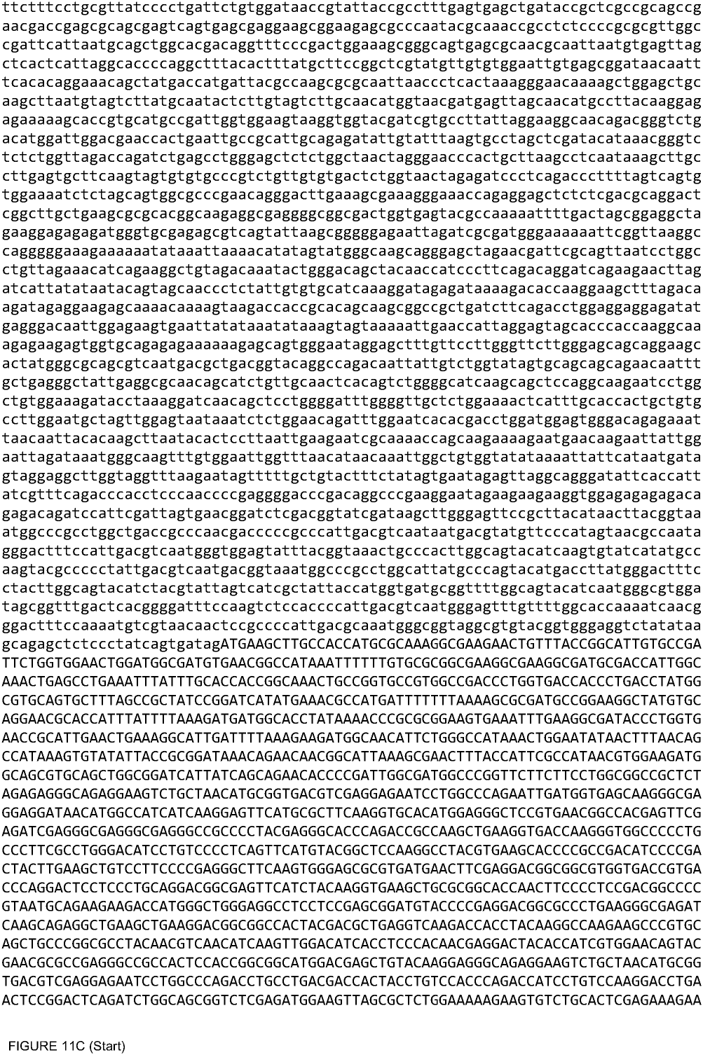

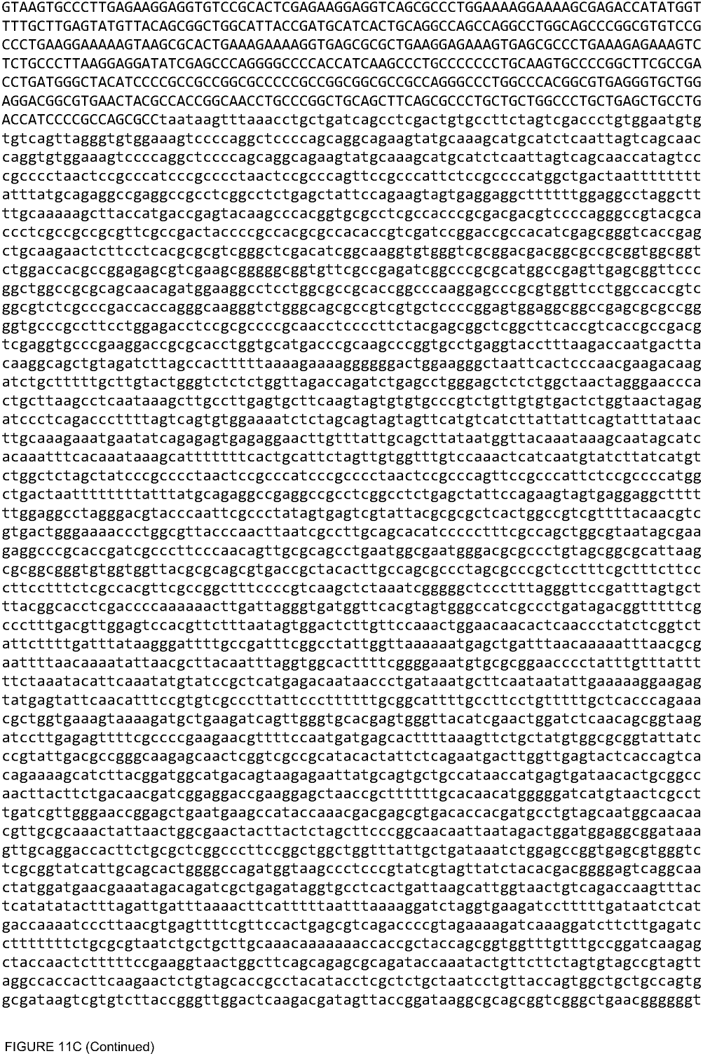

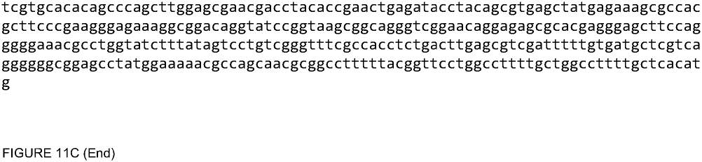

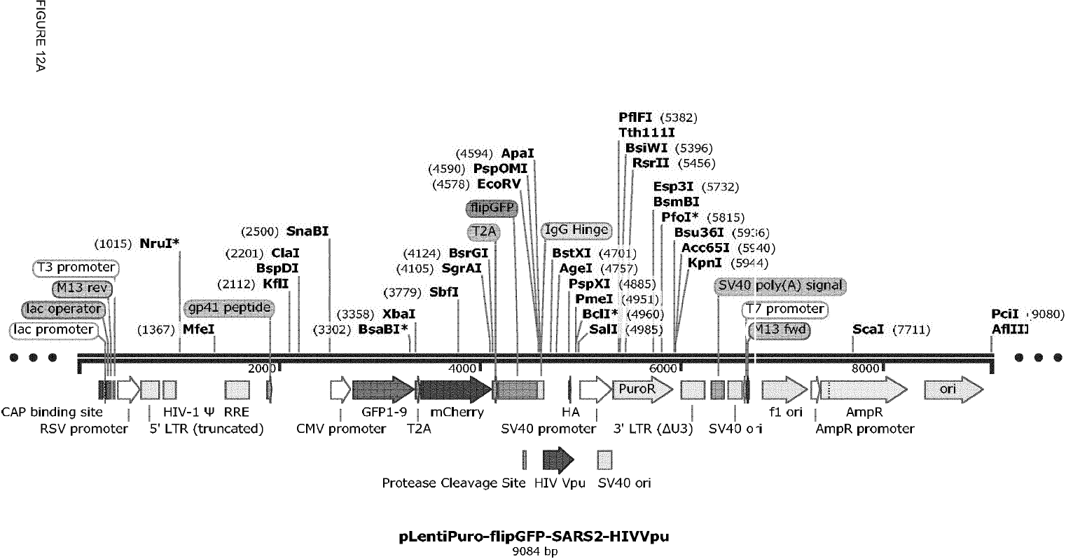

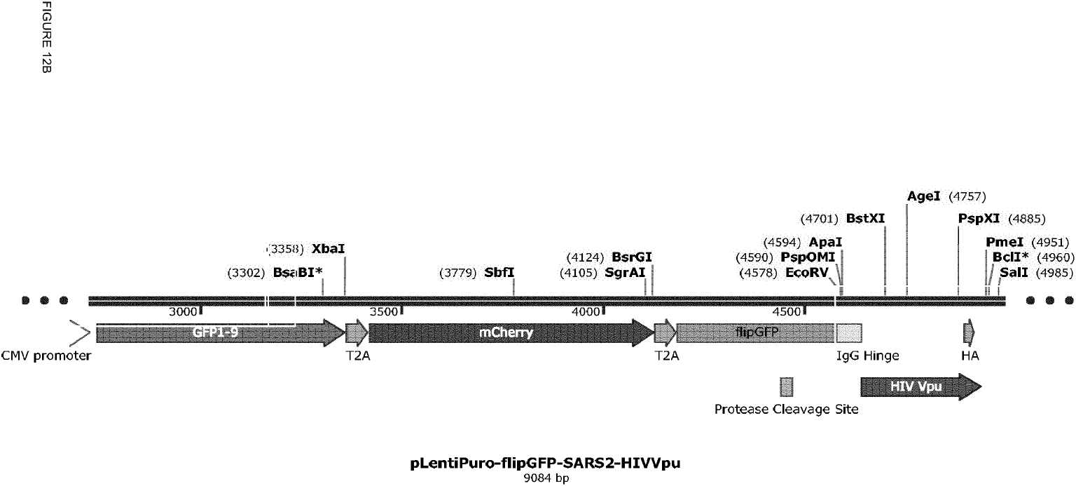

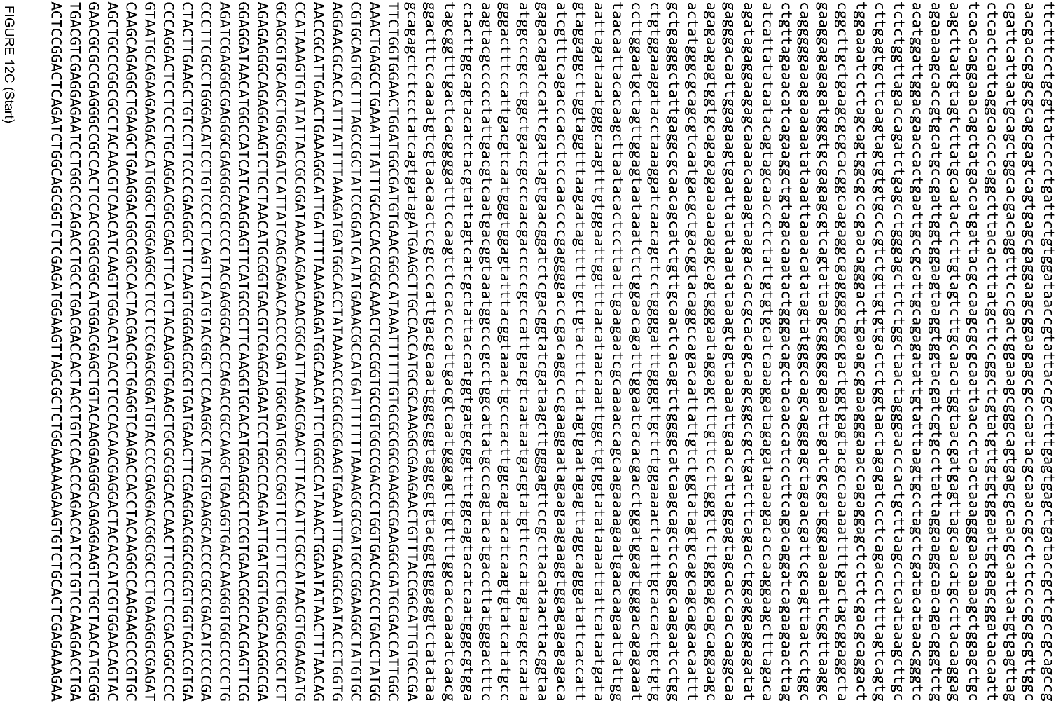

The protocol for transfection and infection of cells has been described in the legends of the figures and the results of the detection are provided on FIGS. 8, 9 and 10.

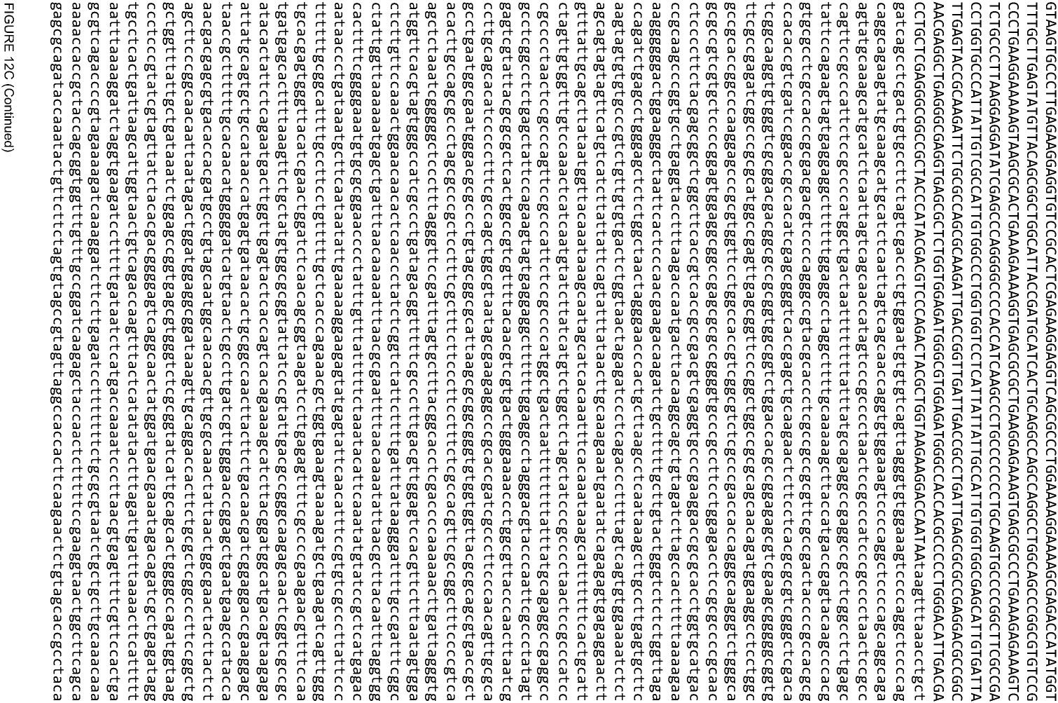

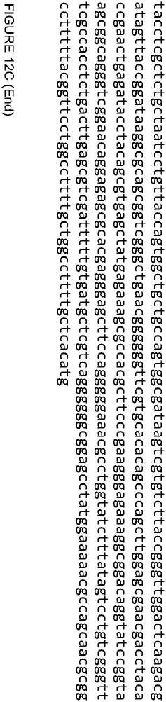

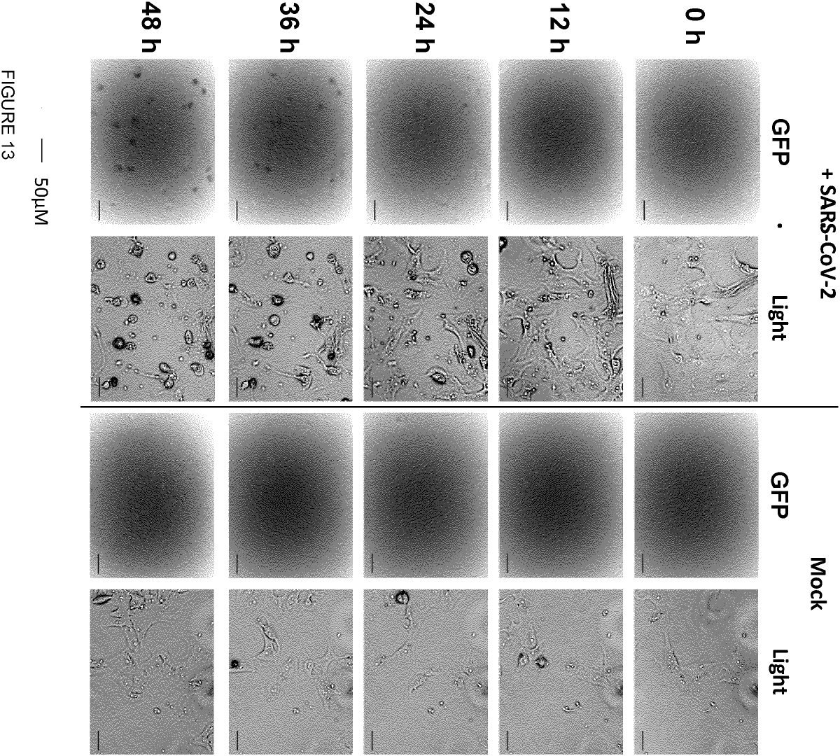

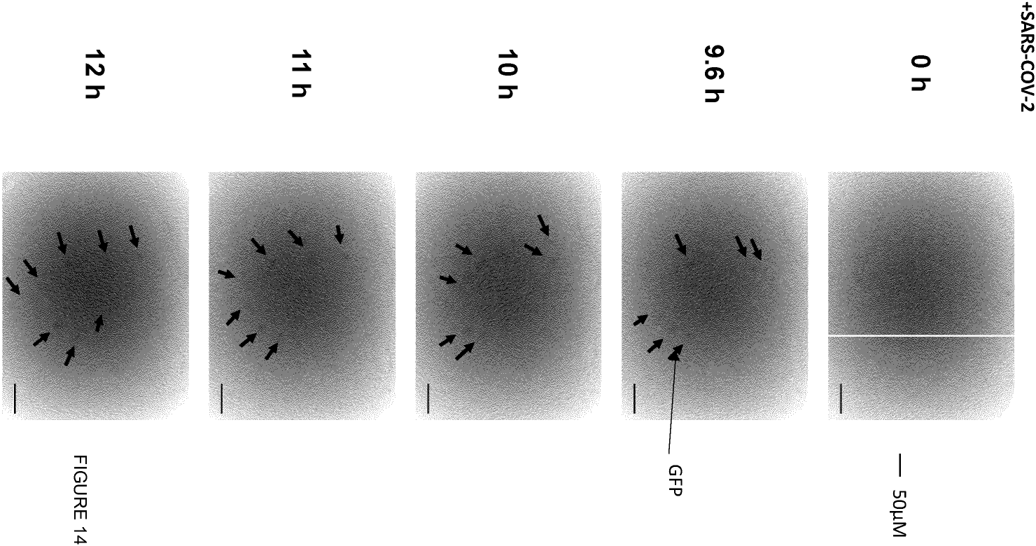

This protocol was repeated with VeroE6 cells that were transfected with flipGFP-SARS2-10-HCV plasmids. These cells were then either infected with SARS-CoV-2 or Mock infected and followed over time in 12-hour intervals to give an overview (FIG. 13). Alternatively, the GFP channel alone was followed at shorter time intervals to focus on early detection (FIG. 14). The white arrows show GFP signal of these cells. These experiments show that virus infected cells can be detected early after infection, especially as early as about 9 hours post infection and that in average such detection may be achieved 16 hours after infection.

The results show that the use of flipGFP for the detection of virus infections work in principle. The activation and generation of a GFP signal is achieved by cleavage events during viral infection, wherein the events are carried out by the viral protease. The specificity and sensitivity of the protease cleavage peptide can be altered by its sequence as well as overall accessibility of the peptide to the viral protease. Many viral proteases appear to be located around replication sites, where the enzymes process the viral polyproteins into functional peptides. Accordingly, at the same time, this allows for targeting of these specific replication sites for a viral reporter system, such as the use of flipGFP. It was possible to identify the endoplasmatic reticulum (ER) as such an important site inside the cell, which brings the reporter and the viral protease in close proximity for cleavage and subsequent activation.

Location of the flipGFP reporter to subcellular compartments, such as the ER, can be achieved by either the use of signal peptides that transport the reporter to this site and retain it there, or by the use of transmembrane peptides of known proteins to be inserted in individual organelle membrane, such as using the HCV Core transmembrane domain for the targeting and insertion into the ER membrane. The cleaved flipGFP peptide can disperse in the cell and bind to the GFP1-10 peptide to form an active, fluorescent GFP protein.

The flipGFP reporter can be cleaved upon the recognition by viral proteases. This can occur within few hours following viral infection. However, this signal detection depends on sensitivity of the system used, such as sensitivity of microscopes. The detection can be mitigated by the increase of the overall expression level of flipGFP in the cell. Thus, the detection of GFP can be shifted to earlier time points, when more of the reporter flipGFP is present in the cell. At the same time, more sensitive systems than the microscope used, including flow cytometry methods, may achieve superior results and limits of detection. The experiments show that detection is possible within a few hours after infection, here as early as about 9 hours post infection with SARS-CoV-2 in VeroE6 cells. The signal in early to mid-stages of infection seem to be specific to the viral protease and can easily be differentiated from control samples. Late events during infection, such as the induction of apoptosis, and therefore activation of caspases, seem to be able to activate the flipGFP reporter in a rather unspecific manner. This allows for a time window of specific flipGFP activation of at least several hours, in VeroE6 cells infected with SARS-CoV-2 such as about 8-14 hours. However, this could be mitigated in systems that either use cells that do not easily undergo apoptosis during viral infection or by the use of molecules that prevent caspase activation.

TABLE 1

| | | |
|---|---|---|
| SEQ ID No. and Name | 5′-3′ Sequence | Purpose |
| 1.pLenti-Puro Linear For | GACGAGCTGTACAAGTAATAACACATCGACAATCAACCTCTGG | Linearizing plasmid for InFusion Cloning to insert flipGFP |
| 2.pLenti-Puro Linear Rev | CGTCAGGCAGGTCCATGGATCCCGTACGCCCGGGCGGTGTC | Linearizing plasmid for InFusion Cloning to insert flipGFP |
| 3.flipGFP For | ATGGACCTGCCTGACGACCACTAC | Preparing flipGFP for Infusion Cloning |
| 4.flipGFP Rev | TTACTTGTACAGCTCGTCCATGCC | Preparing flipGFP for Infusion Cloning |
| 5.Membrane For | GCTGCTGCTTCAGCAAGACCATGGACCTGCCTGACGACCACTAC | Inserting Membrane Signal Peptide into pLenti-Puro plasmid |
| 6.Membrane Rev | CTTGCTGAAGCAGCAGCCCATGGATCCCGTACGCCCGGGCGG | Inserting Membrane Signal Peptide into pLenti-Puro plasmid |
| 7.pLenti-Puro ER For | CACGGCGGCGGCAAGATGGACCTGCCTGACGACCAC | Linearizing plasmid for InFusion Cloning to insert ER Retention Signal Peptide |
| 8.pLenti-Puro ER Rev | CCACCACGGGGTCCATGGATCCCGTACGCCCGGGCGG | Linearizing plasmid for InFusion Cloning to insert ER Retention Signal Peptide |
| 9.ER Signal For | ATGGACCCCGTGGTGGTGCTGGGCCTGTGCCTGAGCTGCCTGCTGCTGCTGAGCCTGTGGAAGCAGAGCCACGGCGGCGGCAAG | Inserting ER Retention Signal Peptide into pLenti-Puro plasmid |
| 10.ER Signal Rev | CTTGCCGCCGCCGTGGCTCTGCTTCCACAGGCTCAGCAGCAGCAGGCAGCTCAGGCACAGGCCCAGCACCACCACGGGGTCCAT | Inserting ER Retention Signal Peptide into pLenti-Puro plasmid |
| 11.SARS2-1 For | CGGCGGCGCCTACACCAGGTACGTGAAGGTGTCCGCCCTGAAGGAAAAAG | Generation of flipGFP recognized by SARS-CoV-2 Nsp3 protease |
| 12.SARS2-1 Rev | GGTGTAGGCGCCGCCGTTCAGCTCCCTTGATGCATCGGTAATGCCAGCCGC | Generation of flipGFP recognized by SARS-CoV-2 Nsp3 protease |
| 13.SARS2-2 For | GGGCGGCGCCCCCACCAAGGTGACCAAGGTGTCCGCCCTGAAGGAAAAAG | Generation of flipGFP recognized by SARS-CoV-2 Nsp3 protease |
| 14.SARS2-2 Rev | GGTGGGGGCGCCGCCCTTCAGGGTGAATGATGCATCGGTAATGCCAGCCGC | Generation of flipGFP recognized by SARS-CoV-2 Nsp3 protease |
| 15.SARS2-3 For | GAAGGGCGGCAAGATCGTGAACAACTGGAAGGTGTCCGCCCTGAAGGAAAAAG | Generation of flipGFP recognized by SARS-CoV-2 Nsp5 protease |
| 16.SARS2-3 Rev | CACGATCTTGCCGCCCTTCAGGGCGATTGATGCATCGGTAATGCCAGCCGC | Generation of flipGFP recognized by SARS-CoV-2 Nsp5 protease |
| 17.SARS2-4 For | GTGCTGCAGAGCGGCTTCAGGAAGATGAAGGTGTCCGCCCTGAAGGAAAAAG | Generation of flipGFP recognized by SARS-CoV-2 Nsp5 protease |
| 18.SARS2-4 Rev | CTGAAGCCGCTCTGCAGCACGGCGCTGGTTGATGCATCGGTAATGCCAGCCGC | Generation of flipGFP recognized by SARS-CoV-2 Nsp5 protease |
| 19.SARS2-5 For | GACCTTCCAGAGCGCCGTGAAGAGGACCAAGGTGTCCGCCCTGAAGGAAAAAG | Generation of flipGFP recognized by SARS-CoV-2 Nsp5 protease |
| 20.SARS2-5 Rev | CACGGCGCTCTGGAAGGTCACGCCGCTTGATGCATCGGTAATGCCAGCCGC | Generation of flipGFP recognized by SARS-CoV-2 Nsp5 protease |
| 21.SARS2-6 For | CACCGTGCAGAGCAAGATGAGCGACGTGAAGGTGTCCGCCCTGAAGGAAAAAG | Generation of flipGFP recognized by SARS-CoV-2 Nsp5 protease |
| 22.SARS2-6 Rev | CATCTTGCTCTGCACGGTGGCCACCTTTGATGCATCGGTAATGCCAGCCGC | Generation of flipGFP recognized by SARS-CoV-2 Nsp5 protease |
| 23.SARS2-7 For | GAAGCTGCAGAACAACGAGCTGAGCCCCAAGGTGTCCGCCCTGAAGGAAAAAG | Generation of flipGFP recognized by SARS-CoV-2 Nsp5 protease |
| 24.SARS2-7 Rev | GCTCGTTGTTCTGCAGCTTCACGGCGCTTGATGCATCGGTAATGCCAGCCGC | Generation of flipGFP recognized by SARS-CoV-2 Nsp5 protease |
| 25.SARS2-8 For | GAGGCTGCAGGCCGGCAACGCCACCGAGAAGGTGTCCGCCCTGAAGGAAAAAG | Generation of flipGFP recognized by SARS-CoV-2 Nsp5 protease |
| 26.SARS2-8 Rev | GTTGCCGGCCTGCAGCCTCACGGTGGCTGATGCATCGGTAATGCCAGCCGC | Generation of flipGFP recognized by SARS-CoV-2 Nsp5 protease |
| 27.SARS2-9 For | CATGCTGCAGAGCGCCGACGCCCAGAGCAAGGTGTCCGCCCTGAAGGAAAAAG | Generation of flipGFP recognized by SARS-CoV-2 Nsp5 protease |
| 28.SARS2-9 Rev | GTCGGCGCTCTGCAGCATGGGCTCCCTTGATGCATCGGTAATGCCAGCCGC | Generation of flipGFP recognized by SARS-CoV-2 Nsp5 protease |
| 29.ZIKV-1 For | GGAAGAGGAGGGGCGCCGACACCAGCATCAAGGTGTCCGCCCTGAAGGAAAAAG | Generation of flipGFP recognized by ZIKV NS2B-NS3 protease |
| 30.ZIKV-1 Rev | GTCGGCGCCCCTCCTCTTCCTCTCCTTTGATGCATCGGTAATGCCAGCCGC | Generation of flipGFP recognized by ZIKV NS2B-NS3 protease |
| 31.ZIKV-2 For | GCGGCAAGAGGAGCTGGCCCCCCAGCGAGAAGGTGTCCGCCCTGAAGGAAAAAG | Generation of flipGFP recognized by ZIKV NS2B-NS3 protease |
| 32.ZIKV-2 Rev | GGGGCCAGCTCCTCTTGCCGCTCCTGGTTGATGCATCGGTAATGCCAGCCGC | Generation of flipGFP recognized by ZIKV NS2B-NS3 protease |
| 33.ZIKV-3 For | GAAGCAGAGGAGCCCCCAGGACAACCAGAAGGTGTCCGCCCTGAAGGAAAAAG | Generation of flipGFP recognized by ZIKV NS2B-NS3 protease |
| 34.ZIKV-3 Rev | CTGGGGGCTCCTCTGCTTCTCGGGCTCTGATGCATCGGTAATGCCAGCCGC | Generation of flipGFP recognized by ZIKV NS2B-NS3 protease |
| 35.ZIKV-4 For | GTGAAGAGGAGGGGCGGCGGCACCGGCGAGAAGGTGTCCGCCCTGAAGGAAAAAG | Generation of flipGFP recognized by ZIKV NS2B-NS3 protease |
| 36.ZIKV-4 Rev | GCCGCCGCCCCTCCTCTTCACCAGGCCTGATGCATCGGTAATGCCAGCCGC | Generation of flipGFP recognized by ZIKV NS2B-NS3 protease |
| 37.CHKV-1 For | GGGCCGGCGCCGGCATCATCGAGACCCCCAAGGTGTCCGCCCTGAAGGAAAAAG | Generation of flipGFP recognized by CHKV Nsp2 protease |
| 38.CHKV-1 Rev | GATGATGCCGGCGCCGGCCCTGTCCTCTGATGCATCGGTAATGCCAGCCGC | Generation of flipGFP recognized by CHKV Nsp2 protease |
| 39.CHKV-2 For | CACCCTGTAGCTGGGGGCGCAGCCGGCCCAAGGTGTCCGCCCTGAAGGAAAAAG | Generation of flipGFP recognized by CHKV Nsp2 protease |
| 40.CHKV-2 Rev | GCTGGGGGCGCAGCCGGCCCTGGTGGCTGATGCATCGGTAATGCCAGCCGC | Generation of flipGFP recognized by CHKV Nsp2 protease |
| 41.CHKV-3 For | GGGCCGGCGGCTACATCTTCAGCAGCGACAAGGTGTCCGCCCTGAAGGAAAAAG | Generation of flipGFP recognized by CHKV Nsp2 protease |
| 42.CHKV-3 Rev | GAAGATGTAGCCGCCGGCCCTGTCCAGTGATGCATCGGTAATGCCAGCCGC | Generation of flipGFP recognized by CHKV Nsp2 protease |
| 43.EV71-2A For | CACTCTTGGGAAATTTGGACAAAAGGTGTCCGCCCTGAAGGAAAAAG | Generation of flipGFP recognized by EV71 2A protease |
| 44.EV71-2A Rev | CAAATTTCCCAAGAGTGGTGATTGATGCATCGGTAATGCCAGCCGC | Generation of flipGFP recognized by EV71 2A protease |
| 45.EV71-3C For | CGCCAGCGAGCAGGGCGAGATCCAGTGGGTGAAGGTGTCCGCCCTGAAGGAAAAAG | Generation of flipGFP recognized by EV71 3C protease |
| 46.EV71-3C Rev | GATCTCGCCCTGCTCGCTGGCGAAGTAGCTTGATGCATCGGTAATGCCAGCCGC | Generation of flipGFP recognized by EV71 3C protease |

The sequences contained in the sequence listing are numbered as follows:

| (SEQ ID) | Nom |
|---|---|
| 1 | pLenti-Puro Linear For |
| 2 | pLenti-Puro Linear Rev |
| 3 | flipGFP For |
| 4 | flipGFP Rev |
| 5 | Membrane For (nt) |
| 6 | Membrane Rev (nt) |
| 7 | pLenti-Puro ER For (nt) |
| 8 | pLenti-Puro ER Rev (nt) |
| 9 | ER Signal For (nt) |
| 10 | ER Signal Rev (nt) |
| 11 | SARS2-1 For (nt) |
| 12 | SARS2-1 Rev (nt) |
| 13 | SARS2-2 For (nt) |
| 14 | SARS2-2 Rev (nt) |
| 15 | SARS2-3 For (nt) |
| 16 | SARS2-3 Rev (nt) |
| 17 | SARS2-4 For (nt) |
| 18 | SARS2-4 Rev (nt) |
| 19 | SARS2-5 For (nt) |
| 20 | SARS2-5 Rev (nt) |
| 21 | SARS2-6 For (nt) |
| 22 | SARS2-6 Rev (nt) |
| 23 | SARS2-7 For (nt) |
| 24 | SARS2-7 Rev (nt) |
| 25 | SARS2-8 For (nt) |
| 26 | SARS2-8 Rev (nt) |
| 27 | SARS2-9 For (nt) |
| 28 | SARS2-9 Rev (nt) |
| 29 | ZIKV-1 For (nt) |
| 30 | ZIKV-1 Rev (nt) |
| 31 | ZIKV-2 For (nt) |
| 32 | ZIKV-2 Rev (nt) |
| 33 | ZIKV-3 For (nt) |
| 34 | ZIKV-3 Rev (nt) |
| 35 | ZIKV-4 For (nt) |
| 36 | ZIKV-4 Rev (nt) |
| 37 | CHKV-1 For (nt) |
| 38 | CHKV-1 Rev (nt) |
| 39 | CHKV-2 For (nt) |
| 40 | CHKV-2 Rev (nt) |
| 41 | CHKV-3 For (nt) |
| 42 | CHKV-3 Rev (nt) |
| 43 | EV71-2A For (nt) |
| 44 | EV71-2A Rev (nt) |
| 45 | EV71-3C For (nt) |
| 46 | EV71-3C Rev (nt) |
| 47 | flipGFP_CHKV_1 (nt) |
| 48 | flipGFP_CHKV_2 (nt) |
| 49 | flipGFP_CHKV_3 (nt) |
| 50 | flipGFP_ER_CHKV_1 (nt) |
| 51 | flipGFP_ER_CHKV_2 (nt) |
| 52 | flipGFP_ER_CHKV_3 (nt) |
| 53 | flipGFP_ER_EV71_2A (nt) |
| 54 | flipGFP_ER_EV71_3C (nt) |
| 55 | flipGFP_ER_SARSCoV2_1 (nt) |
| 56 | flipGFP_ER_SARSCoV2_2 (nt) |
| 57 | flipGFP_ER_SARSCoV2_3 (nt) |
| 58 | flipGFP_ER_SARSCoV2_4 (nt) |
| 59 | flipGFP_ER_SARSCoV2_5 (nt) |
| 60 | flipGFP_ER_SARSCoV2_6 (nt) |
| 61 | flipGFP_ER_SARSCoV2_7 (nt) |
| 62 | flipGFP_ER_SARSCoV2_8 (nt) |
| 63 | flipGFP_ER_SARSCoV2_9 (nt) |
| 64 | flipGFP_ER_ZIKV_1 (nt) |
| 65 | flipGFP_ER_ZIKV_2 (nt) |
| 66 | flipGFP_ER_ZIKV_3 (nt) |
| 67 | flipGFP_ER_ZIKV_4 (nt) |
| 68 | flipGFP_EV71_2A (nt) |
| 69 | flipGFP_EV71_3C (nt) |
| 70 | flipGFP_Membrane_CHKV_1 (nt) |
| 71 | flipGFP_Membrane_CHKV_2 (nt) |
| 72 | flipGFP_Membrane_CHKV_3 (nt) |
| 73 | flipGFP_Membrane_EV71_2A (nt) |
| 74 | flipGFP_Membrane_EV71_3C (nt) |
| 75 | flipGFP_Membrane_SARSCoV2_1 (nt) |
| 76 | flipGFP_Membrane_SARSCoV2_2 (nt) |
| 77 | flipGFP_Membrane_SARSCoV2_3 (nt) |
| 78 | flipGFP_Membrane_SARSCoV2 4 (nt) |
| 79 | flipGFP_Membrane_SARSCoV2_5 (nt) |
| 80 | flipGFP_Membrane_SARSCoV2_6 (nt) |
| 81 | flipGFP_Membrane SARSCoV2 7 (nt) |
| 82 | flipGFP_Membrane_SARSCoV2 8 (nt) |
| 83 | flipGFP_Membrane_SARSCoV2_9 (nt) |
| 84 | flipGFP_Membrane_ZIKV_1 (nt) (nt) |
| 85 | flipGFP_Membrane_ZIKV_2 (nt) |
| 86 | flipGFP_Membrane_ZIKV_3 (nt) |
| 87 | flipGFP_Membrane_ZIKV_4 (nt) |
| 88 | flipGFP_SARSCoV2_1 (nt) |
| 89 | flipGFP_SARSCoV2_2 (nt) |
| 90 | flipGFP_SARSCoV2_3 (nt) |
| 91 | flipGFP_SARSCoV2_4 (nt) |
| 92 | flipGFP_SARSCoV2_5 (nt) |
| 93 | flipGFP_SARSCoV2_6 (nt) |
| 94 | flipGFP_SARSCoV2_7 (nt) |
| 95 | flipGFP_SARSCoV2_8 (nt) |
| 96 | flipGFP_SARSCoV2_9 (nt) |
| 97 | flipGFP_ZIKV_1 (nt) |
| 98 | flipGFP_ZIKV_2 (nt) |
| 99 | flipGFP_ZIKV_3 (nt) |
| 100 | flipGFP_ZIKV_4 (nt) |
| 101 | pLentiPuro_flipGFP_ER_3C(nt) |
| 102 | pLentiPuro_flipGFP_Membrane_3C (nt) |
| 103 | pLentiPuro-flipGFP-3C (nt) |
| 104 | ER retention signal (nt) |
| 105 | ER retention signal/1 (aa) |
| 106 | Membrane targeting signal (nt) |
| 107 | Membrane targeting signal (aa) |
| 108 | cleavage site for for CHKV-1 (nt) |
| 109 | cleavage site for for CHKV-2 |
| 110 | cleavage site for for CHKV-3 |
| 111 | EV71_2A |
| 112 | EV71_3C |
| 113 | SARSCoV2_1 |
| 114 | SARSCoV2_2 |
| 115 | SARSCoV2_3 |
| 116 | SARSCoV2_4 |
| 117 | SARSCoV2_5 |
| 118 | SARSCoV2_6 |
| 119 | SARSCoV2_7 |
| 120 | SARSCoV2_8 |
| 121 | SARSCoV2_9 |
| 122 | ZIKV_1 |
| 123 | ZIKV_2 |
| 124 | ZIKV_3 |
| 125 | ZIKV_4 |
| 126 | Enterovirus 2A protease cleavage site (aa) |
| 127 | Enterovirus 3C protease cleavage site (aa) |
| 128 | Enterovirus 3C protease cleavage site (aa) |
| 129 | Alphaviruses protease cleavage site (aa) |
| 130 | SARS-CoV-2 protease cleavage site (aa) |
| 131 | SARS-CoV-2 protease cleavage site/1 (aa) |
| 132 | SARS-CoV-2 protease cleavage site/2 (aa) |
| 133 | SARS-CoV-2 protease cleavage site/3 (aa) |
| 134 | SARS-CoV-2 protease cleavage site/4 (aa) |
| 135 | SARS-CoV-2 protease cleavage site/5 (aa) |
| 136 | SARS-CoV-2 protease cleavage site/6 (aa) |
| 137 | SARS-CoV-2 protease cleavage site/7 (aa) |
| 138 | SARS-CoV-2 protease cleavage site/8 (aa) |
| 139 | SARS virus protease cleavage site (aa) |
| 140 | Flavivirus protease cleavage site (aa) |
| 141 | ZIK viruses protease cleavage site (aa) |
| 142 | ZIK viruses protease cleavage site/1 (aa) |
| 143 | ZIK viruses protease cleavage site/2 (aa) |
| 144 | ZIK viruses protease cleavage site/3 (aa) |
| 145 | FlipGFP |
| 146 | mCherry (nt) |
| 147 | 2A peptide from Thosea asigna virus capsid |
| 148 | GFP1-9 |
| 149 | Flipped GFP domains with protease cleavage site for SARS-2 protease |
| 150 | pLentiPuro-flipGFP-SARS2-HCVCore |
| 151 | pLentiPuro-flipGFP-SARS2-HIVVpu |
| 152 | HCV Core TM |
| 153 | HIV Vpu |

| | |
|---|---|
| 47 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGAGGACAGGGCCGGCGCCGGCATCATCGAGACCCCCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 48 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGCCACCAGGGCCGGCTGCGCCCCCAGCTACAGGGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 49 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCACTGGACAGGGCCGGCGGCTACATCTTCAGCAGCGACAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 50 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGAGGACAGGGCCGGCGCCGGCATCATCGAGACCCCCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 51 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGCCACCAGGGCCGGCTGCGCCCCCAGCTACAGGGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 52 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCACTGGACAGGGCCGGCGGCTACATCTTCAGCAGCGACAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 53 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAATCACCACTCTTGGGAAATTTGGACAAAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 54 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGCTACTTCGCCAGCGAGCAGGGCGAGATCCAGTGGGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 55 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGGGAGCTGAACGGCGGCGCCTACACCAGGTACGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGG CATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 56 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCATTCACCCTGAAGGGCGGCGCCCCCACCAAGGTGACCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 57 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAATCGCCCTGAAGGGCGGCAAGATCGTGAACAACTGGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 58 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAACCAGCGCCGTGCTGCAGAGCGGCTTCAGGAAGATGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 59 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGCGGCGTGACCTTCCAGAGCGCCGTGAAGAGGACCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 60 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAAGGTGGCCACCGTGCAGAGCAAGATGAGCGACGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 61 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGCGCCGTGAAGCTGCAGAACAACGAGCTGAGCCCCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 62 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGCCACCGTGAGGCTGCAGGCCGGCAACGCCACCGAGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 63 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGGGAGCCCATGCTGCAGAGCGCCGACGCCCAGAGCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 64 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAAGGAGAGGAAGAGGAGGGGCGCCGACACCAGCATCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 65 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAACCAGGAGCGGCAAGAGGAGCTGGCCCCCCAGCGAGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 66 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGAGCCCGAGAAGCAGAGGAGCCCCCAGGACAACCAGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 67 | ATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGGCCTGGTGAAGAGGAGGGGCGGCGGCACCGGCGAGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 68 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAATCACCACTCTTGGGAAATTTGGACAAAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 69 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGCTACTTCGCCAGCGAGCAGGGCGAGATCCAGTGGGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 70 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGAGGACAGGGCCGGCGCCGGCATCATCGAGACCCCCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 71 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGCCACCAGGGCCGGCTGCGCCCCCAGCTACAGGGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 72 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCACTGGACAGGGCCGGCGGCTACATCTTCAGCAGCGACAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 73 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAATCACCACTCTTGGGAAATTTGGACAAAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 74 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGCTACTTCGCCAGCGAGCAGGGCGAGATCCAGTGGGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 75 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGGGAGCTGAACGGCGGCGCCTACACCAGGTACGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 76 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCATTCACCCTGAAGGGCGGCGCCCCCACCAAGGTGACCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 77 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAATCGCCCTGAAGGGCGGCAAGATCGTGAACAACTGGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 78 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAACCAGCGCCGTGCTGCAGAGCGGCTTCAGGAAGATGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 79 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGCGGCGTGACCTTCCAGAGCGCCGTGAAGAGGACCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 80 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAAGGTGGCCACCGTGCAGAGCAAGATGAGCGACGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 81 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGCGCCGTGAAGCTGCAGAACAACGAGCTGAGCCCCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 82 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGCCACCGTGAGGCTGCAGGCCGGCAACGCCACCGAGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 83 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGGGAGCCCATGCTGCAGAGCGCCGACGCCCAGAGCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 84 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAAGGAGAGGAAGAGGAGGGGCGCCGACACCAGCATCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 85 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAACCAGGAGCGGCAAGAGGAGCTGGCCCCCCAGCGAGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 86 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGAGCCCGAGAAGCAGAGGAGCCCCCAGGACAACCAGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 87 | ATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGGCCTGGTGAAGAGGAGGGGCGGCGGCACCGGCGAGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 88 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGGGAGCTGAACGGCGGCGCCTACACCAGGTACGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 89 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCATTCACCCTGAAGGGCGGCGCCCCCACCAAGGTGACCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 90 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAATCGCCCTGAAGGGCGGCAAGATCGTGAACAACTGGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGG CGATGGCCCGGTTCTTCTTCCTGGCGGCCGCTCTAGA |
| 91 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAACCAGCGCCGTGCTGCAGAGCGGCTTCAGGAAGATGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 92 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGCGGCGTGACCTTCCAGAGCGCCGTGAAGAGGACCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 93 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAAGGTGGCCACCGTGCAGAGCAAGATGAGCGACGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 94 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGCGCCGTGAAGCTGCAGAACAACGAGCTGAGCCCCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 95 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGCCACCGTGAGGCTGCAGGCCGGCAACGCCACCGAGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 96 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGGGAGCCCATGCTGCAGAGCGCCGACGCCCAGAGCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 97 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAAGGAGAGGAAGAGGAGGGGCGCCGACACCAGCATCAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 98 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAACCAGGAGCGGCAAGAGGAGCTGGCCCCCCAGCGAGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 99 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGAGCCCGAGAAGCAGAGGAGCCCCCAGGACAACCAGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 100 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAGGCCTGGTGAAGAGGAGGGGCGGCGGCACCGGCGAGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGAGAGGGCAGAGGAAGTCTGCTAACATGCGGTGACGTCGAGGAGAATCCTGGCCCAGAATTGATGGTGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAGTTCATCTACAAGGTGAAGCTGCGCGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTGAGGTCAAGACCACCTACAAGGCCAAGAAGCCCGTGCAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGtaataaCACatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgfttgctgacgcaacccccactggttggggcattgccaccacctgtcagctcctttccgggactttcgctttccccctccctattgccacggcggaactcatcgccgcctgccttgcccgctgctggacaggggctcggctgftgggcactgacaattccgtggtgttgtcggggaagctgacgtcctttccatggctgctcgcctgtgttgccacctggattctgcgcgggacgtccttctgctacgtcccttcgGccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt |
| 101 | aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgggagttccgcgttacataacttacggtaaatggcccgcctggctgaccgcccaacgacccccgcccattgacgtcaataatgacgtatgttcccatagtaacgccaatagggactftccaftgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcccgggcgtacgggatccATGGACCCCGTGGTGGTGCTGGGCCTGTGCCTGAGCTGCCTGCTGCTGCTGAGCCTGTGGAAGCAGAGCCACGGCGGCGGCAAGATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGCTACTTCGCCAGCGAGCAGGGCGAGATCCAGTGGGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCCAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGAGAGGGCAGAGGAAGTCTGCTAACATGCGGTGACGTCGAGGAGAATCCTGGCCCAGAATTGATGGTGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAGTTCATCTACAAGGTGAAGCTGCGCGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTGAGGTCAAGACCACCTACAAGGCCAAGAAGCCCGTGCAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGtaataaCACatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccaccctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtattttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaatttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgcccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattccctttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaa ttaatagactggatggaggcggataaagttgcaggaccacttctgcgctcggcccttccggctggctggtttattgctgataaatctggagccggtgagcgtgggtctgcggtatcattgcagcactggggccagatggtaagccctcccgtatcgtagttatctacacgacggggagtcaggcaactatggatgaacgaaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctcccgcgcgttggccgattcattaatgcagctggcacgacaggtttcccactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt |
| 102 | aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcccgggcgtacgggatccATGGGCTGCTGCTTCAGCAAGACCATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGCTACTTCGCCAGCGAGCAGGGCGAGATCCAGTGGGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAATTTTTTGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGAGAGGGCAGAGGAAGTCTGCTAACATGCGGTGACGTCGAGGAGAATCCTGGCCCAGAATTGATGGTGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAG TTCATCTACAAGGTGAAGCTGCGCGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTGAGGTCAAGACCACCTACAAGGCCAAGAAGCCCGTGCAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGtaataaCACatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgccatccegeoccctaactcegceccagttcecgeoccattctccgecccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTGAGGTCAAGACCACCTACAAGGCCAAGAAGCCCGTGCAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGtaataaCACatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcttgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt |
| 103 | aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagcaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcccgggcgtacgggatccATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAGCTACTTCGCCAGCGAGCAGGGCGAGATCCAGTGGGTGAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAACTGGATGGCGATGTGAACGGCCATAAAITTTTIGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGAGAGGGCAGAGGAAGTCTGCTAACATGCGGTGACGTCGAGGAGAATCCTGGCCCAGAATTGATGGTGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAGTTCATCTACAAGGTGAAGCTGCGCGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTTGAGGTCAAGACCACCTACAAGGCCAAGAAGCCCGTGCAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGtaataaCACatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggcgcctccccgcctggaattcgagctcggtacctttaagaccaatgacttacaaggcagctgtagatcttagccactttttaaaagaaaaggggggactggaaggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattccctttttgcggcattttgccttcctgtttttgctcacccagaaacgctggtgaaagtaaaagatgctgaagatcagttgggtgcacgagtgggttacatcgaactggatctcaacagcggtaagatccttgaggttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt |
| 104 | ATGGACCCCGTGGTGGTGCTGGGCCTGTGCCTGAGCTGCCTGCTGCTGCTGAGCCTGTGGAAGCAGAGCCACGGCGGCGGCAAG |
| 105 | MDPVVVLGLCLSCLLLLSLWQSHGGGK |
| 106 | ATGGGCTGCTGCTTCAGCAAGACC |
| 107 | MGCCFSKT |
| 108 | GAGGACAGGGCCGGCGCCGGCATCATCGAGACCCCC |
| 109 | GCCACCAGGGCCGGCTGCGCCCCCAGCTACAGGGTG |
| 110 | CTGGACAGGGCCGGCGGCTACATCTTCAGCAGCGAC |
| 111 | ATCACCACTCTTGGGAAATTTGGACAA |
| 112 | AGCTACTTCGCCAGCGAGCAGGGCGAGATCCAGTGGGTG |
| 113 | AGGGAGCTGAACGGCGGCGCCTACACCAGGTACGTG |
| 114 | TTCACCCTGAAGGGCGGCGCCCCCACCAAGGTGACC |
| 115 | ATCGCCCTGAAGGGCGGCAAGATCGTGAACAACTGG |
| 116 | ACCAGCGCCGTGCTGCAGAGCGGCTTCAGGAAGATG |
| 117 | AGCGGCGTGACCTTCCAGAGCGCCGTGAAGAGGACC |
| 118 | AAGGTGGCCACCGTGCAGAGCAAGATGAGCGACGTG |
| 119 | AGCGCCGTGAAGCTGCAGAACAACGAGCTGAGCCCC |
| 120 | GCCACCGTGAGGCTGCAGGCCGGCAACGCCACCGAG |
| 121 | AGGGAGCCCATGCTGCAGAGCGCCGACGCCCAGAGC |
| 122 | AAGGAGAGGAAGAGGAGGGGCGCCGACACCAGCATC |
| 123 | ACCAGGAGCGGCAAGAGGAGCTGGCCCCCCAGCGAG |
| 124 | GAGCCCGAGAAGCAGAGGAGCCCCCAGGACAACCAG |
| 125 | GGCCTGGTGAAGAGGAGGGGCGGCGGCACCGGCGAG |
| 126 | ITTLGKFGQ |
| 127 | EALFQGPK |
| 128 | SYFASEQGEIQWV |
| 129 | RAGAYIFS |
| 130 | RELNGGAYTRYV |
| 131 | FTLKGGAPTKVT |
| 132 | IALKGGKIVNNW |
| 133 | TSAVLQSGFRKM |
| 134 | KVATVQSKMSDV |
| 135 | SAVKLQNNELSP |
| 136 | ATVRLQAGNATE |
| 137 | REPMLQSADAQS |
| 138 | SGVTFQSAVKRT |
| 139 | SGVTFQGKFKK |
| 140 | YAKRGGVF |
| 141 | KERKRRGADTSI |
| 142 | TRSGKRSWPPSE |
| 143 | EPEKQRSPQDNQ |
| 144 | GLVKRRGGGTGE |
| 145 | ATGGACCTGCCTGACGACCACTACCTGTCCACCCAGACCATCCTGTCCAAGGACCTGAACTCCGGACTCAGATCTGGCAGCGGTCTCGAGATGGAAGTTAGCGCTCTGGAAAAAGAAGTGTCTGCACTCGAGAAAGAAGTAAGTGCCCTTGAGAAGGAGGTGTCCGCACTCGAGAAGGAGGTCAGCGCCCTGGAAAAGGAAAAGCGAGACCATATGGTTTTGCTTGAGTATGTTACAGCGGCTGGCATTACCGATGCATCAAAGGTGTCCGCCCTGAAGGAAAAAGTAAGCGCACTGAAAGAAAAGGTGAGCGCGCTGAAGGAGAAAGTGAGCGCCCTGAAAGAGAAAGTCTCTGCCCTTAAGGAGGATATCGAGGGCAGAGGAAGTCTGCTAACATGCGGTGACGTCGAGGAGAATCCTGGCCCAAAGCTTGCCACCATGCGCAAAGGCGAAGAACTGTTTACCGGCATTGTGCCGATTCTGGTGGAAC TGGATGGCGATGTGAACGGCCATAAATTTTTTIGTGCGCGGCGAAGGCGAAGGCGATGCGACCATTGGCAAACTGAGCCTGAAATTTATTTGCACCACCGGCAAACTGCCGGTGCCGTGGCCGACCCTGGTGACCACCCTGACCTATGGCGTGCAGTGCTTTAGCCGCTATCCGGATCATATGAAACGCCATGATTTTTTTAAAAGCGCGATGCCGGAAGGCTATGTGCAGGAACGCACCATTTATTTTAAAGATGATGGCACCTATAAAACCCGCGCGGAAGTGAAATTTGAAGGCGATACCCTGGTGAACCGCATTGAACTGAAAGGCATTGATTTTAAAGAAGATGGCAACATTCTGGGCCATAAACTGGAATATAACTTTAACAGCCATAAAGTGTATATTACCGCGGATAAACAGAACAACGGCATTAAAGCGAACTTTACCATTCGCCATAACGTGGAAGATGGCAGCGTGCAGCTGGCGGATCATTATCAGCAGAACACCCCGATTGGCGATGGCCCGGTTCTTCTTCCTGGCGGCCGCTCTAGA |
| 146 | TGAGCAAGGGCGAGGAGGATAACATGGCCATCATCAAGGAGTTCATGCGCTTCAAGGTGCACATGGAGGGCTCCGTGAACGGCCACGAGTTCGAGATCGAGGGCGAGGGCGAGGGCCGCCCCTACGAGGGCACCCAGACCGCCAAGCTGAAGGTGACCAAGGGTGGCCCCCTGCCCTTCGCCTGGGACATCCTGTCCCCTCAGTTCATGTACGGCTCCAAGGCCTACGTGAAGCACCCCGCCGACATCCCCGACTACTTGAAGCTGTCCTTCCCCGAGGGCTTCAAGTGGGAGCGCGTGATGAACTTCGAGGACGGCGGCGTGGTGACCGTGACCCAGGACTCCTCCCTGCAGGACGGCGAGTTCATCTACAAGGTGAAGCTGCGCGGCACCAACTTCCCCTCCGACGGCCCCGTAATGCAGAAGAAGACCATGGGCTGGGAGGCCTCCTCCGAGCGGATGTACCCCGAGGACGGCGCCCTGAAGGGCGAGATCAAGCAGAGGCTGAAGCTGAAGGACGGCGGCCACTACGACGCTGAGGTCAAGACCACCTACAAGGCCAAGAAGCCCGTGCAGCTGCCCGGCGCCTACAACGTCAACATCAAGTTGGACATCACCTCCCACAACGAGGACTACACCATCGTGGAACAGTACGAACGCGCCGAGGGCCGCCACTCCACCGGCGGCATGGACGAGCTGTACAAGtaa |
| 147 | GAGGGCAGAGGAAGTCTGCTAACATGCGGTGACGTCGAGGAGAATCCTGGCCCA |

1. A nucleic acid construct which comprises an operon wherein the operon comprises (a) a recombinant transgene that encodes a recombinant inactive form of a fluorescent reporter protein, and a cleavage site for a viral protease, and (b) a nucleic acid coding for a detectable expression control protein, wherein the nucleic acid sequences of (a) and (b) are operably assembled in said operon under the control of a single promoter and optionally of additional control sequence(s) for transcription and/or translation and wherein the nucleic acid sequences of (a) and (b) are optionally separated by the sequence of a polyprotein separating site such as the sequence of the separating 2A-peptide originating from Thosea asigna virus capsid such as the sequence of SEQ ID No.147.

2. The nucleic acid construct according to claim 1, wherein the recombinant transgene encoding the inactive form of the fluorescent reporter protein comprises a nucleotide sequence of an altered form of the Open Reading Frame (ORF) that encodes the active form of the fluorescent reporter protein and wherein said alteration in the ORF comprises switching position in the ORF of at least one nucleotide sequence encoding specific structure domains of the active form of the fluorescent protein to prevent assembly of the expressed structure domains as a functional protein enabling maturation of the chromophore and wherein the nucleotide sequence encoding the inactive form of the fluorescent reporter protein additionally comprises a nucleotide sequence encoding a cleavage site for a determined protease.

3. The nucleic acid construct according to claim 2, wherein the nucleic acid coding for a detectable expression control protein is interposed within the sequence encoding the structural domains of the sequence of the active form of a fluorescent reporter protein.

4. The nucleic acid according to any one of claims 1 to 3, wherein the active fluorescent reporter protein is an active flipGFP and the inactive fluorescent reporter protein is an inactive flipGFP.

5. The nucleic acid construct according to claim 4, wherein the recombinant transgene comprises from 5′-end to 3′-end polynucleotides encoding the beta10 strand of flipGFP, a linker having from 3 to 15, in particular about 10, amino acid residues, the E5 domain of flipGFP, the beta11 strand of flipGFP, the cleavage site of the viral protease and the K5 domain of the flipGFP, the sequence of a polyprotein separating site such as the sequence of the separating 2A-peptide originating from Thosea asigna virus capsid, a polynucleotide encoding the beta1-9 strand of flipGFP wherein these polynucleotides together encode the inactive form of the recombinant flipGFP with the viral protease cleavage site.

6. The nucleic acid construct according to claim 3, which comprises from 5′-end to 3′-end polynucleotides encoding the beta1-9 strand of flipGFP, the sequence of a polyprotein separating site such as the sequence of the separating 2A-peptide originating from Thosea asigna virus capsid, the nucleic acid coding for a detectable expression control protein, polynucleotides encoding the beta10 strand of flipGFP, a linker having from 3 to 15, in particular about 10, amino acid residues, the E5 domain of flipGFP, the beta11 strand of flipGFP, the cleavage site of the viral protease and the K5 domain of the flipGFP, wherein these polynucleotides together encode the inactive form of the recombinant flipGFP with the viral protease cleavage site and the detectable expression control protein.

7. The nucleic acid construct according to any one of claims 1 to 6, wherein the transgene further comprises upstream from the sequence encoding the inactive fluorescent reporter protein in particular the inactive flipGFP, a sequence encoding a signal peptide, in particular a signal peptide for retention of the expressed polypeptides in the endoplasmic reticulum (ER retention signal) or a signal peptide for targeting the expressed polypeptides to the cell membrane (membrane targeting signal), especially a Cytochrome P450 ER retention signal of sequence MDPVVVLGLCLSCLLLLSLWQSHGGGK (SEQ ID No.105) or a membrane targeting signal of sequence MGCCFSKT (SEQ ID No.107)..

8. The nucleic acid construct according to any one of claims 1 to 6, which further comprises operably associated with the sequence encoding the inactive fluorescent reporter protein in particular the inactive flipGFP, a sequence encoding a peptide for retention of the expressed part of the fluorescent reporter polypeptide associated thereto in the endoplasmic reticulum (ER retention signal) or for targeting the expressed polypeptides into the ER membrane or the cell membrane.

9. The nucleic acid construct according to claim 7 or 8, which comprises a nucleotide sequence encoding the HCV Core TM peptide that is the sequence of SEQ ID No.152) or encoding the HIV Vpu peptide that is the sequence SEQ ID No.153.

10. The nucleic acid construct according to claim 7, wherein the polynucleotide encoding the signal peptide contains or consists of the sequence of ATGGACCCCGTGGTGGTGCTGGGCCTGTGCCTGAGCTGCCTGCTGCTGCTGAG CCTGTGGAAGCAGAGCCACGGCGGCGGCAAG (SEQ ID No.104) encoding the Cytochrome P450 ER retention signal peptide or contains or consists of the sequence of ATGGGCTGCTGCTTCAGCAAGACC (SEQ ID No.106) encoding the membrane targeting signal peptide.

11. The nucleic acid construct according to claim 9, wherein the polynucleotide encoding the transmembrane peptide contains or consists of the sequence of SEQ ID No.152 encoding the HCV Core TM peptide for retention into the ER membrane, or the sequence of SEQ ID No.153 encoding the HIV Vpu peptide and this sequence is operably associated with the inactive flipGFP.

12. The nucleic acid construct according to any one of claims 1 to 11, wherein the expression control protein is a fluorescent protein with a fluorophore of a color that is different from the color of the reporter fluorescent protein, in particular is mCherry protein or mTurquoise protein or cyan fluorescent protein (ECFP), yellow fluorescent protein such as mVenus, in particular the nucleic acid construct contains the polynucleotide of SEQ ID No.146 coding for mCherry.

13. The nucleic acid construct according to any one of claims 1 to 12, wherein the cleavage site is recognized by a protease of a determined virus family selected in the group of Alphaviruses, Coronaviruses, Enteroviruses, Retroviruses and Flaviviruses.

14. The nucleic acid construct according to any one of claims 1 to 13, wherein the nucleic acid sequence for the protease cleavage site encodes an amino acid sequence selected from the group of ITTLGKFGQ (SEQ ID No.126) for the Enterovirus 2A protease, EALFQGPK (SEQ ID No.127) or SYFASEQGEIQWV (SEQ ID No.128) for the Enterovirus 3C protease, RAGAYIFS (SEQ ID No.129) for Alphaviruses, RELNGGAYTRYV (SEQ ID No.130), FTLKGGAPTKVT (SEQ ID No.131), IALKGGKIVNNW (SEQ ID No.132), TSAVLQSGFRKM (SEQ ID No.133), KVATVQSKMSDV (SEQ ID No.134), SAVKLQNNELSP (SEQ ID No.135), ATVRLQAGNATE (SEQ ID No.136), REPMLQSADAQS (SEQ ID No.137), SGVTFQSAVKRT (SEQ ID No.138) for SARS-CoV-2 coronavirus, SGVTFQGKFKK (SEQ ID No.139) for SARS virus coronavirus, YAKRGGVF (SEQ ID No140) for Flaviviruses, and more particularly KERKRRGADTSI (SEQ ID No.141), TRSGKRSWPPSE (SEQ ID No.142), EPEKQRSPQDNQ (SEQ ID No.143), GLVKRRGGGTGE (SEQ ID No.144) for ZIKA viruses.

15. The nucleic acid construct according to any one of claims 1 to 13, wherein the polynucleotide encoding the viral protease cleavage site consists of a polynucleotide selected from the group of:
GAGGACAGGGCCGGCGCCGGCATCATCGAGACCCCC for CHKV-1 (SEQ ID No.108) or GCCACCAGGGCCGGCTGCGCCCCCAGCTACAGGGTG for CHKV-2 (SEQ ID No.109) or CTGGACAGGGCCGGCGGCTACATCTTCAGCAGCGAC for CHKV-3 (SEQ ID No.110) or,
ATCACCACTCTTGGGAAATTTGGACAA for EV71_2A (SEQ ID No.111) or AGCTACTTCGCCAGCGAGCAGGGCGAGATCCAGTGGGTG for EV71_3C (SEQ ID No.112) or,
AGGGAGCTGAACGGCGGCGCCTACACCAGGTACGTG for SARSCoV2_1 (SEQ ID No.113) or TTCACCCTGAAGGGCGGCGCCCCCACCAAGGTGACC for SARSCoV2_2 (SEQ ID No.114) or ATCGCCCTGAAGGGCGGCAAGATCGTGAACAACTGG for SARSCoV2_3 (SEQ ID No.115) or ACCAGCGCCGTGCTGCAGAGCGGCTTCAGGAAGATG for SARSCoV2_4 (SEQ ID No.116) or AGCGGCGTGACCTTCCAGAGCGCCGTGAAGAGGACC for SARSCoV2_5 (SEQ ID No.117) or AAGGTGGCCACCGTGCAGAGCAAGATGAGCGACGTG for SARSCoV2_6 (SEQ ID No.118) or AGCGCCGTGAAGCTGCAGAACAACGAGCTGAGCCCC for SARSCoV2_7 (SEQ ID No.119) or GCCACCGTGAGGCTGCAGGCCGGCAACGCCACCGAG for SARSCoV2_8 (SEQ ID No.120) or AGGGAGCCCATGCTGCAGAGCGCCGACGCCCAGAGC for SARSCoV2_9 (SEQ ID No.121) or,
AAGGAGAGGAAGAGGAGGGGCGCCGACACCAGCATC for ZIKV_1 (SEQ ID No.122) or ACCAGGAGCGGCAAGAGGAGCTGGCCCCCCAGCGAG for ZIKV_2 (SEQ ID No.123) or GAGCCCGAGAAGCAGAGGAGCCCCCAGGACAACCAG for ZIKV_3 (SEQ ID No.124) or GGCCTGGTGAAGAGGAGGGGCGGCGGCACCGGCGAG for ZIKV_4 (SEQ ID No.125).

16. The nucleic acid construct according to any one of claims 4 to 15, wherein the flipGFP is encoded by the sequence of SEQ ID No.145 and optionally wherein the sequence of the cleavage site for the protease is inserted at position 262 in said sequence.

17. The nucleic acid construct according to any one of claims 1 to 16, wherein the polynucleotide that encodes the inactive recombinant flipGFP is selected from the group of SEQ ID No 47 to SEQ ID No.100, SEQ ID No. 150 and SEQ ID No.151 respectively for flipGFP_CHKV_1, flipGFP_CHKV_2, flipGFP_CHKV_3, flipGFP_ER_CHKV_1, flipGFP_ER_CHKV_2, flipGFP_ER_CHKV_3, flipGFP_ER_EV71_2A, flipGFP_ER_EV71_3C, flipGFP_ER_SARSCoV2_1, flipGFP_ER_SARSCoV2_2, flipGFP_ER_SARSCoV2_3, flipGFP_ER_SARSCoV2_4, flipGFP_ER_SARSCoV2_5, flipGFP_ER_SARSCoV2_6, flipGFP_ER_SARSCoV2_7, flipGFP_ER_SARSCoV2_8, flipGFP_ER_SARSCoV2_9, flipGFP_ER_ZIKV_1, flipGFP_ER_ZIKV_2, flipGFP_ER_ZIKV_3, flipGFP_ER_ZIKV_4, flipGFP_EV71_2A, flipGFP_EV71_3C, flipGFP_Membrane_CHKV_1, flipGFP_Membrane_CHKV_2, flipGFP_Membrane_CHKV_3, flipGFP_Membrane_EV71_2A, flipGFP_Membrane_EV71_3C, flipGFP_Membrane_SARSCoV2_1, flipGFP_Membrane_SARSCoV2_2, flipGFP_Membrane_SARSCoV2_3, flipGFP_Membrane_SARSCoV2_4, flipGFP_Membrane_SARSCoV2_5, flipGFP_Membrane_SARSCoV2_6, flipGFP_Membrane_SARSCoV2_7, flipGFP_Membrane_SARSCoV2_8, flipGFP_Membrane_SARSCoV2_9, flipGFP_Membrane_ZIKV_1, flipGFP_Membrane_ZIKV_2, flipGFP_Membrane_ZIKV_3, flipGFP_Membrane_ZIKV_4, flipGFP_SARSCoV2_1, flipGFP_SARSCoV2_2, flipGFP_SARSCoV2_3, flipGFP_SARSCoV2_4, flipGFP_SARSCoV2_5, flipGFP_SARSCoV2_6, flipGFP_SARSCoV2_7, flipGFP_SARSCoV2_8, flipGFP_SARSCoV2_9, flipGFP_ZIKV_1, flipGFP_ZIKV_2, flipGFP_ZIKV_3, flipGFP_ZIKV_4, flipGFP-SARS2-HCVCore, and flipGFP-SARS2-HIVVpu.

18. The recombinant nucleic acid according to any one of claims 1 to 14, which is selected from the group of pLentiPuro_flipGFP_ER_3C (SEQ ID No.101), pLentiPuro_flipGFP_Membrane_3C (SEQ ID No.102), and pLentiPuro-flipGFP-3C (SEQ ID No.103).

19. The recombinant nucleic acid according to any one of claims 1, 8, 9 or 11 to 18, which is selected from the group of pLentiPuro-flipGFP-SARS2-HCVCore (SEQ ID No.150), and pLentiPuro-flipGFP-SARS2-HIVVpu (SEQ ID No.151).

20. A set of at least two nucleic acid constructs according to any one of claims 1 to 19.

21. The nucleic acid construct according to any one of claims 1 to 20, wherein the promoter of the transgene is active in cells selected from the group of prokaryotic cells, in particular in bacterial cells or in archaeal cells, and/or is active in eukaryotic cells, in particular in mammalian cells or in insect cells, in particular the promoter is the CMV promoter.

22. A transformation vector which comprises a nucleic acid construct according to any one of claims 1 to 21, in particular a vector which is a plasmid for transfection or for transduction, especially a lentiviral vector plasmid.

23. A cell which is a prokaryotic or a eukaryotic cell or cell line transformed with the nucleic acid construct according to any one of claims 1 to 130or with a transformation vector according to claim 22, in particular a stable cell line, especially a stable cell line transduced with lentiviral vector particles expressing a nucleic acid construct according to any one of claims 1 to 21.

24. The cell according to claim 23, which is a cell selected for its sensibility to infection by a determined human virus targeting the cleavage site of the inactive fluorescent protein expressed in the cell, wherein the cell is optionally a stable cell line.

25. The cell according to claim 23 or 24, which is a cell line stably expressing the nucleic acid construct according to any one of claims 1 to 21, wherein the nucleic acid construct is inserted in the genome of the cell.

26. A polynucleotide encoding a viral protease cleavage site which is selected from the group of GAGGACAGGGCCGGCGCCGGCATCATCGAGACCCCC for CHKV-1 (SEQ ID No.108) or GCCACCAGGGCCGGCTGCGCCCCCAGCTACAGGGTG for CHKV-2 (SEQ ID No.109) or CTGGACAGGGCCGGCGGCTACATCTTCAGCAGCGAC for CHKV-3 (SEQ ID No.110) or,
ATCACCACTCTTGGGAAATTTGGACAA for EV71_2A (SEQ ID No.111) or AGCTACTTCGCCAGCGAGCAGGGCGAGATCCAGTGGGTG for EV71_3C (SEQ ID No.112) or,
AGGGAGCTGAACGGCGGCGCCTACACCAGGTACGTG for SARSCoV2_1 (SEQ ID No.113) or TTCACCCTGAAGGGCGGCGCCCCCACCAAGGTGACC for SARSCoV2_2 (SEQ ID No.114) or ATCGCCCTGAAGGGCGGCAAGATCGTGAACAACTGG for SARSCoV2_3 (SEQ ID No.115) or ACCAGCGCCGTGCTGCAGAGCGGCTTCAGGAAGATG for SARSCoV2_4 (SEQ ID No.116) or AGCGGCGTGACCTTCCAGAGCGCCGTGAAGAGGACC for SARSCoV2_5 (SEQ ID No.117) or AAGGTGGCCACCGTGCAGAGCAAGATGAGCGACGTG for SARSCoV2_6 (SEQ ID No.118) or AGCGCCGTGAAGCTGCAGAACAACGAGCTGAGCCCC for SARSCoV2_7 (SEQ ID No.119) or GCCACCGTGAGGCTGCAGGCCGGCAACGCCACCGAG for SARSCoV2_8 (SEQ ID No.120) or AGGGAGCCCATGCTGCAGAGCGCCGACGCCCAGAGC for SARSCoV2_9 (SEQ ID No.121) or,
AAGGAGAGGAAGAGGAGGGGCGCCGACACCAGCATC for ZIKV_1 (SEQ ID No.122) or ACCAGGAGCGGCAAGAGGAGCTGGCCCCCCAGCGAG for ZIKV_2 (SEQ ID No.123) or GAGCCCGAGAAGCAGAGGAGCCCCCAGGACAACCAG for ZIKV_3 (SEQ ID No.124) or GGCCTGGTGAAGAGGAGGGGCGGCGGCACCGGCGAG for ZIKV_4 (SEQ ID No.125).

27. Use of a nucleic acid construct according to any one of claims 1 to 21 or of a vector according to claim 22, for in vitro detection and optionally quantification of a viral infection in a biological sample of a human or animal subject, wherein the detection targets a virus recognizing the cleavage site recombined in the inactive fluorescent reporter protein.

28. Use of a cell line according to claim 25, for in vitro detection and optionally quantification of a viral infection in a biological sample of a human or animal subject, wherein the detection targets a virus recognizing the cleavage site recombined in the inactive fluorescent reporter protein.

29. The use according to claim 27 or 28, wherein fluorescence of the reporter protein is detected or measured directly on the cells.

30. An in vitro method of detecting or monitoring a pathogen infection, in particular a virus infection, in a biological sample previously obtained from a human or an animal subject, which comprises the steps of:
a. Providing cells according to claim 23 to 25 that express either transiently or stably an inactive fluorescent reporter protein comprising a cleavage site for a protease of the virus to be detected,
b. Contacting said cells with the assayed biological sample in conditions that enable the virus when present in the sample, to infect the cells and the viral protease to cleave the protease cleavage site,
c. Allowing fluorescence to increase in the cells that have been contacted with the biological sample in b., following activation of the inactive fluorescent reporter protein by cleavage in step b. of the viral protease cleavage site and measuring said fluorescence of the reporter protein,
d. Optionally comparing the fluorescence level of the active fluorescent reporter protein to a standard or fluorescent control protein expressed in the cells and optionally concluding on virus infectious activity in the subject and/or quantitating the virus.

31. An in vitro method of detecting or monitoring a virus infection, in a biological sample previously obtained from a human or an animal subject, which comprises the steps of:
a. Providing an inactive fluorescent protein as a reporter protein expressed from the nucleic acid construct of any one of claims 1 to 21, together with a control protein expressed from the same nucleic acid construct wherein the inactive fluorescent reporter protein comprises a cleavage site for a protease of the virus to be detected,
b. Contacting said inactive fluorescent reporter protein with the assayed biological sample in conditions that enable the virus protease to target and to cleave the protease cleavage site in the inactive fluorescent reporter protein,
c. Allowing fluorescence to increase in the biological sample following activation of the inactive fluorescent reporter protein by cleavage in step b. and measuring said fluorescence,
d. Optionally comparing the fluorescence level to a standard or to the fluorescence of the control protein and optionally concluding on the virus infectious activity in the subject and/or quantitating the virus.

32. A method of any one of claims 30 or 31 of detecting or monitoring a virus infection, in a biological sample previously obtained from a human or an animal subject, wherein the virus is a determined virus selected in the group of Alphaviruses, Coronaviruses, Enteroviruses, Retroviruses and Flaviviruses, in particular is a coronavirus, especially is SARS-CoV-2 (or SARS-2) responsible for Covid-19.

33. A method according to any one of claims 30 to 32, wherein detection is carried out as soon as 24 hours, in particular as soon as 14 hours, more particularly within a range of 8 to 24 hours, following suspicion of infection.

34. A laboratory animal for experimental or clinical observation of the response to a virus infection, wherein the animal has been transformed to enable its genome to express a nucleic acid construct according to any one of claims 1 to 21 either transiently or stably, the animal being in particular a rodent, an insect or a non-human mammal.


SEQUENCE LISTING (153 sequences)

SEQ ID NO: 1 - DNA, 43 bp, Artificial Sequence
  Synthetic sequence
gacgagctgtacaagtaataacacatcgacaatcaacctctgg

SEQ ID NO: 2 - DNA, 41 bp, Artificial Sequence
  Synthetic sequence
cgtcaggcaggtccatggatcccgtacgcccgggcggtgtc

SEQ ID NO: 3 - DNA, 24 bp, Artificial Sequence
  Synthetic sequence
atggacctgcctgacgaccactac

SEQ ID NO: 4 - DNA, 24 bp, Artificial Sequence
  Synthetic sequence
ttacttgtacagctcgtccatgcc

SEQ ID NO: 5 - DNA, 44 bp, Artificial Sequence
  Synthetic sequence
gctgctgcttcagcaagaccatggacctgcctgacgaccactac

SEQ ID NO: 6 - DNA, 42 bp, Artificial Sequence
  Synthetic sequence
cttgctgaagcagcagcccatggatcccgtacgcccgggcgg

SEQ ID NO: 7 - DNA, 36 bp, Artificial Sequence
  Synthetic sequence
cacggcggcggcaagatggacctgcctgacgaccac

SEQ ID NO: 8 - DNA, 37 bp, Artificial Sequence
  Synthetic sequence
ccaccacggggtccatggatcccgtacgcccgggcgg

SEQ ID NO: 9 - DNA, 84 bp, Artificial Sequence
  Synthetic sequence
atggaccccgtggtggtgctgggcctgtgcctgagctgcctgctgctgctgagcctgtggaagcagagccacggcggcggcaag

SEQ ID NO: 10 - DNA, 84 bp, Artificial Sequence
  Synthetic sequence
cttgccgccgccgtggctctgcttccacaggctcagcagcagcaggcagctcaggcacaggcccagcaccaccacggggtccat

SEQ ID NO: 11 - DNA, 50 bp, Artificial Sequence
  Synthetic sequence
cggcggcgcctacaccaggtacgtgaaggtgtccgccctgaaggaaaaag

SEQ ID NO: 12 - DNA, 51 bp, Artificial Sequence
  Synthetic sequence
ggtgtaggcgccgccgttcagctcccttgatgcatcggtaatgccagccgc

SEQ ID NO: 13 - DNA, 50 bp, Artificial Sequence
  Synthetic sequence
gggcggcgcccccaccaaggtgaccaaggtgtccgccctgaaggaaaaag

SEQ ID NO: 14 - DNA, 51 bp, Artificial Sequence
  Synthetic sequence
ggtgggggcgccgcccttcagggtgaatgatgcatcggtaatgccagccgc

SEQ ID NO: 15 - DNA, 53 bp, Artificial Sequence
  Synthetic sequence
gaagggcggcaagatcgtgaacaactggaaggtgtccgccctgaaggaaaaag

SEQ ID NO: 16 - DNA, 51 bp, Artificial Sequence
  Synthetic sequence
cacgatcttgccgcccttcagggcgattgatgcatcggtaatgccagccgc

SEQ ID NO: 17 - DNA, 52 bp, Artificial Sequence
  Synthetic sequence
gtgctgcagagcggcttcaggaagatgaaggtgtccgccctgaaggaaaaag

SEQ ID NO: 18 - DNA, 53 bp, Artificial Sequence
  Synthetic sequence
ctgaagccgctctgcagcacggcgctggttgatgcatcggtaatgccagccgc

SEQ ID NO: 19 - DNA, 53 bp, Artificial Sequence
  Synthetic sequence
gaccttccagagcgccgtgaagaggaccaaggtgtccgccctgaaggaaaaag

SEQ ID NO: 20 - DNA, 51 bp, Artificial Sequence
  Synthetic sequence
cacggcgctctggaaggtcacgccgcttgatgcatcggtaatgccagccgc

SEQ ID NO: 21 - DNA, 53 bp, Artificial Sequence
  Synthetic sequence
caccgtgcagagcaagatgagcgacgtgaaggtgtccgccctgaaggaaaaag

SEQ ID NO: 22 - DNA, 51 bp, Artificial Sequence
  Synthetic sequence
catcttgctctgcacggtggccacctttgatgcatcggtaatgccagccgc

SEQ ID NO: 23 - DNA, 53 bp, Artificial Sequence
  Synthetic sequence
gaagctgcagaacaacgagctgagccccaaggtgtccgccctgaaggaaaaag

SEQ ID NO: 24 - DNA, 52 bp, Artificial Sequence
  Synthetic sequence
gctcgttgttctgcagcttcacggcgcttgatgcatcggtaatgccagccgc

SEQ ID NO: 25 - DNA, 53 bp, Artificial Sequence
  Synthetic sequence
gaggctgcaggccggcaacgccaccgagaaggtgtccgccctgaaggaaaaag

SEQ ID NO: 26 - DNA, 51 bp, Artificial Sequence
  Synthetic sequence
gttgccggcctgcagcctcacggtggctgatgcatcggtaatgccagccgc

SEQ ID NO: 27 - DNA, 53 bp, Artificial Sequence
  Synthetic sequence
catgctgcagagcgccgacgcccagagcaaggtgtccgccctgaaggaaaaag

SEQ ID NO: 28 - DNA, 51 bp, Artificial Sequence
  Synthetic sequence
gtcggcgctctgcagcatgggctcccttgatgcatcggtaatgccagccgc

SEQ ID NO: 29 - DNA, 54 bp, Artificial Sequence
  Synthetic sequence
ggaagaggaggggcgccgacaccagcatcaaggtgtccgccctgaaggaaaaag

SEQ ID NO: 30 - DNA, 51 bp, Artificial Sequence
  Synthetic sequence
gtcggcgcccctcctcttcctctcctttgatgcatcggtaatgccagccgc

SEQ ID NO: 31 - DNA, 54 bp, Artificial Sequence
  Synthetic sequence
gcggcaagaggagctggccccccagcgagaaggtgtccgccctgaaggaaaaag

SEQ ID NO: 32 - DNA, 52 bp, Artificial Sequence
  Synthetic sequence
ggggccagctcctcttgccgctcctggttgatgcatcggtaatgccagccgc

SEQ ID NO: 33 - DNA, 53 bp, Artificial Sequence
  Synthetic sequence
gaagcagaggagcccccaggacaaccagaaggtgtccgccctgaaggaaaaag

SEQ ID NO: 34 - DNA, 51 bp, Artificial Sequence
  Synthetic sequence
ctgggggctcctctgcttctcgggctctgatgcatcggtaatgccagccgc

SEQ ID NO: 35 - DNA, 55 bp, Artificial Sequence
  Synthetic sequence
gtgaagaggaggggcggcggcaccggcgagaaggtgtccgccctgaaggaaaaag

SEQ ID NO: 36 - DNA, 51 bp, Artificial Sequence
  Synthetic sequence
gccgccgcccctcctcttcaccaggcctgatgcatcggtaatgccagccgc

SEQ ID NO: 37 - DNA, 54 bp, Artificial Sequence
  Synthetic sequence
gggccggcgccggcatcatcgagacccccaaggtgtccgccctgaaggaaaaag

SEQ ID NO: 38 - DNA, 51 bp, Artificial Sequence
  Synthetic sequence
gatgatgccggcgccggccctgtcctctgatgcatcggtaatgccagccgc

SEQ ID NO: 39 - DNA, 54 bp, Artificial Sequence
  Synthetic sequence
caccctgtagctgggggcgcagccggcccaaggtgtccgccctgaaggaaaaag

SEQ ID NO: 40 - DNA, 51 bp, Artificial Sequence
  Synthetic sequence
gctgggggcgcagccggccctggtggctgatgcatcggtaatgccagccgc

SEQ ID NO: 41 - DNA, 54 bp, Artificial Sequence
  Synthetic sequence
gggccggcggctacatcttcagcagcgacaaggtgtccgccctgaaggaaaaag

SEQ ID NO: 42 - DNA, 51 bp, Artificial Sequence
  Synthetic sequence
gaagatgtagccgccggccctgtccagtgatgcatcggtaatgccagccgc

SEQ ID NO: 43 - DNA, 47 bp, Artificial Sequence
  Synthetic sequence
cactcttgggaaatttggacaaaaggtgtccgccctgaaggaaaaag

SEQ ID NO: 44 - DNA, 46 bp, Artificial Sequence
  Synthetic sequence
caaatttcccaagagtggtgattgatgcatcggtaatgccagccgc

SEQ ID NO: 45 - DNA, 56 bp, Artificial Sequence
  Synthetic sequence
cgccagcgagcagggcgagatccagtgggtgaaggtgtccgccctgaaggaaaaag

SEQ ID NO: 46 - DNA, 54 bp, Artificial Sequence
  Synthetic sequence
gatctcgccctgctcgctggcgaagtagcttgatgcatcggtaatgccagccgc

SEQ ID NO: 47 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcagaggacagggccggcgccggcatcatcgagacccccaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 48 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcagccaccagggccggctgcgcccccagctacagggtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 49 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcactggacagggccggcggctacatcttcagcagcgacaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 50 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcagaggacagggccggcgccggcatcatcgagacccccaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 51 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcagccaccagggccggctgcgcccccagctacagggtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 52 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcactggacagggccggcggctacatcttcagcagcgacaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 53 - DNA, 1152 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaatcaccactcttgggaaatttggacaaaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 54 - DNA, 1164 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagctacttcgccagcgagcagggcgagatccagtgggtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 55 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagggagctgaacggcggcgcctacaccaggtacgtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 56 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcattcaccctgaagggcggcgcccccaccaaggtgaccaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 57 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaatcgccctgaagggcggcaagatcgtgaacaactggaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 58 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaaccagcgccgtgctgcagagcggcttcaggaagatgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 59 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagcggcgtgaccttccagagcgccgtgaagaggaccaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 60 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaaaggtggccaccgtgcagagcaagatgagcgacgtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 61 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagcgccgtgaagctgcagaacaacgagctgagccccaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 62 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcagccaccgtgaggctgcaggccggcaacgccaccgagaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 63 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagggagcccatgctgcagagcgccgacgcccagagcaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 64 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaaaggagaggaagaggaggggcgccgacaccagcatcaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 65 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaaccaggagcggcaagaggagctggccccccagcgagaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 66 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcagagcccgagaagcagaggagcccccaggacaaccagaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 67 - DNA, 1161 bp, Artificial Sequence
  Synthetic sequence
atggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaggcctggtgaagaggaggggcggcggcaccggcgagaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 68 - DNA, 1068 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaatcaccactcttgggaaatttggacaaaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 69 - DNA, 1080 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagctacttcgccagcgagcagggcgagatccagtgggtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 70 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcagaggacagggccggcgccggcatcatcgagacccccaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 71 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcagccaccagggccggctgcgcccccagctacagggtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 72 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcactggacagggccggcggctacatcttcagcagcgacaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 73 - DNA, 1092 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaatcaccactcttgggaaatttggacaaaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 74 - DNA, 1104 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagctacttcgccagcgagcagggcgagatccagtgggtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 75 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagggagctgaacggcggcgcctacaccaggtacgtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 76 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcattcaccctgaagggcggcgcccccaccaaggtgaccaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 77 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaatcgccctgaagggcggcaagatcgtgaacaactggaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 78 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaaccagcgccgtgctgcagagcggcttcaggaagatgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 79 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagcggcgtgaccttccagagcgccgtgaagaggaccaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 80 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaaaggtggccaccgtgcagagcaagatgagcgacgtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 81 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagcgccgtgaagctgcagaacaacgagctgagccccaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 82 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcagccaccgtgaggctgcaggccggcaacgccaccgagaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 83 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagggagcccatgctgcagagcgccgacgcccagagcaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 84 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaaaggagaggaagaggaggggcgccgacaccagcatcaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 85 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaaccaggagcggcaagaggagctggccccccagcgagaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 86 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcagagcccgagaagcagaggagcccccaggacaaccagaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 87 - DNA, 1101 bp, Artificial Sequence
  Synthetic sequence
atgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaggcctggtgaagaggaggggcggcggcaccggcgagaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 88 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagggagctgaacggcggcgcctacaccaggtacgtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 89 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcattcaccctgaagggcggcgcccccaccaaggtgaccaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 90 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaatcgccctgaagggcggcaagatcgtgaacaactggaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 91 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaaccagcgccgtgctgcagagcggcttcaggaagatgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 92 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagcggcgtgaccttccagagcgccgtgaagaggaccaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 93 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaaaggtggccaccgtgcagagcaagatgagcgacgtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 94 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagcgccgtgaagctgcagaacaacgagctgagccccaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 95 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcagccaccgtgaggctgcaggccggcaacgccaccgagaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 96 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagggagcccatgctgcagagcgccgacgcccagagcaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 97 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaaaggagaggaagaggaggggcgccgacaccagcatcaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 98 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaaccaggagcggcaagaggagctggccccccagcgagaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 99 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcagagcccgagaagcagaggagcccccaggacaaccagaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 100 - DNA, 1077 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaggcctggtgaagaggaggggcggcggcaccggcgagaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 101 - DNA, 8638 bp, Artificial Sequence
  Synthetic sequence
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcccgggcgtacgggatccatggaccccgtggtggtgctgggcctgtgcctgagctgcctgctgctgctgagcctgtggaagcagagccacggcggcggcaagatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagctacttcgccagcgagcagggcgagatccagtgggtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctagagagggcagaggaagtctgctaacatgcggtgacgtcgaggagaatcctggcccagaattgatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaataacacatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 102 - DNA, 8578 bp, Artificial Sequence
  Synthetic sequence
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcccgggcgtacgggatccatgggctgctgcttcagcaagaccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagctacttcgccagcgagcagggcgagatccagtgggtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctagagagggcagaggaagtctgctaacatgcggtgacgtcgaggagaatcctggcccagaattgatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaataacacatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 103 - DNA, 8554 bp, Artificial Sequence
  Synthetic sequence
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaattcaaaattttatcgataagcttgggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgcccgggcgtacgggatccatggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaagctacttcgccagcgagcagggcgagatccagtgggtgaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctagagagggcagaggaagtctgctaacatgcggtgacgtcgaggagaatcctggcccagaattgatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaataacacatcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 104 - DNA, 84 bp, Artificial Sequence; identical to SEQ ID NO: 9
  Synthetic sequence

SEQ ID NO: 105 - protein, 27 aa, Artificial Sequence
  Synthetic sequence
MDPVVVLGLCLSCLLLLSLWQSHGGGK

SEQ ID NO: 106 - DNA, 24 bp, Artificial Sequence
  Synthetic sequence
atgggctgctgcttcagcaagacc

SEQ ID NO: 107 - protein, 8 aa, Artificial Sequence
  Synthetic sequence
MGCCFSKT

SEQ ID NO: 108 - DNA, 36 bp, Artificial Sequence
  Synthetic sequence
gaggacagggccggcgccggcatcatcgagaccccc

SEQ ID NO: 109 - DNA, 36 bp, Artificial Sequence
  Synthetic sequence
gccaccagggccggctgcgcccccagctacagggtg

SEQ ID NO: 110 - DNA, 36 bp, Artificial Sequence
  Synthetic sequence
ctggacagggccggcggctacatcttcagcagcgac

SEQ ID NO: 111 - DNA, 27 bp, Artificial Sequence
  Synthetic sequence
atcaccactcttgggaaatttggacaa

SEQ ID NO: 112 - DNA, 39 bp, Artificial Sequence
  Synthetic sequence
agctacttcgccagcgagcagggcgagatccagtgggtg

SEQ ID NO: 113 - DNA, 36 bp, Artificial Sequence
  Synthetic sequence
agggagctgaacggcggcgcctacaccaggtacgtg

SEQ ID NO: 114 - DNA, 36 bp, Artificial Sequence
  Synthetic sequence
ttcaccctgaagggcggcgcccccaccaaggtgacc

SEQ ID NO: 115 - DNA, 36 bp, Artificial Sequence
  Synthetic sequence
atcgccctgaagggcggcaagatcgtgaacaactgg

SEQ ID NO: 116 - DNA, 36 bp, Artificial Sequence
  Synthetic sequence
accagcgccgtgctgcagagcggcttcaggaagatg

SEQ ID NO: 117 - DNA, 36 bp, Artificial Sequence
  Synthetic sequence
agcggcgtgaccttccagagcgccgtgaagaggacc

SEQ ID NO: 118 - DNA, 36 bp, Artificial Sequence
  Synthetic sequence
aaggtggccaccgtgcagagcaagatgagcgacgtg

SEQ ID NO: 119 - DNA, 36 bp, Artificial Sequence
  Synthetic sequence
agcgccgtgaagctgcagaacaacgagctgagcccc

SEQ ID NO: 120 - DNA, 36 bp, Artificial Sequence
  Synthetic sequence
gccaccgtgaggctgcaggccggcaacgccaccgag

SEQ ID NO: 121 - DNA, 36 bp, Artificial Sequence
  Synthetic sequence
agggagcccatgctgcagagcgccgacgcccagagc

SEQ ID NO: 122 - DNA, 36 bp, Artificial Sequence
  Synthetic sequence
aaggagaggaagaggaggggcgccgacaccagcatc

SEQ ID NO: 123 - DNA, 36 bp, Artificial Sequence
  Synthetic sequence
accaggagcggcaagaggagctggccccccagcgag

SEQ ID NO: 124 - DNA, 36 bp, Artificial Sequence
  Synthetic sequence
gagcccgagaagcagaggagcccccaggacaaccag

SEQ ID NO: 125 - DNA, 36 bp, Artificial Sequence
  Synthetic sequence
ggcctggtgaagaggaggggcggcggcaccggcgag

SEQ ID NO: 126 - protein, 9 aa, Artificial Sequence
  Synthetic sequence
ITTLGKFGQ

SEQ ID NO: 127 - protein, 8 aa, Artificial Sequence
  Synthetic sequence
EALFQGPK

SEQ ID NO: 128 - protein, 13 aa, Artificial Sequence
  Synthetic sequence
SYFASEQGEIQWV

SEQ ID NO: 129 - protein, 8 aa, Artificial Sequence
  Synthetic sequence
RAGAYIFS

SEQ ID NO: 130 - protein, 12 aa, Artificial Sequence
  Synthetic sequence
RELNGGAYTRYV

SEQ ID NO: 131 - protein, 12 aa, Artificial Sequence
  Synthetic sequence
FTLKGGAPTKVT

SEQ ID NO: 132 - protein, 12 aa, Artificial Sequence
  Synthetic sequence
IALKGGKIVNNW

SEQ ID NO: 133 - protein, 12 aa, Artificial Sequence
  Synthetic sequence
TSAVLQSGFRKM

SEQ ID NO: 134 - protein, 12 aa, Artificial Sequence
  Synthetic sequence
KVATVQSKMSDV

SEQ ID NO: 135 - protein, 12 aa, Artificial Sequence
  Synthetic sequence
SAVKLQNNELSP

SEQ ID NO: 136 - protein, 12 aa, Artificial Sequence
  Synthetic sequence
ATVRLQAGNATE

SEQ ID NO: 137 - protein, 12 aa, Artificial Sequence
  Synthetic sequence
REPMLQSADAQS

SEQ ID NO: 138 - protein, 12 aa, Artificial Sequence
  Synthetic sequence
SGVTFQSAVKRT

SEQ ID NO: 139 - protein, 11 aa, Artificial Sequence
  Synthetic sequence
SGVTFQGKFKK

SEQ ID NO: 140 - protein, 8 aa, Artificial Sequence
  Synthetic sequence
YAKRGGVF

SEQ ID NO: 141 - protein, 12 aa, Artificial Sequence
  Synthetic sequence
KERKRRGADTSI

SEQ ID NO: 142 - protein, 12 aa, Artificial Sequence
  Synthetic sequence
TRSGKRSWPPSE

SEQ ID NO: 143 - protein, 12 aa, Artificial Sequence
  Synthetic sequence
EPEKQRSPQDNQ

SEQ ID NO: 144 - protein, 12 aa, Artificial Sequence
  Synthetic sequence
GLVKRRGGGTGE

SEQ ID NO: 145 - DNA, 1041 bp, Artificial Sequence
  Synthetic sequence
atggacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcaaaggtgtccgccctgaaggaaaaagtaagcgcactgaaagaaaaggtgagcgcgctgaaggagaaagtgagcgccctgaaagagaaagtctctgcccttaaggaggatatcgagggcagaggaagtctgctaacatgcggtgacgtcgaggagaatcctggcccaaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 146 - DNA, 707 bp, Artificial Sequence
  Synthetic sequence
tgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 147 - DNA, 54 bp, Artificial Sequence
  Synthetic sequence
gagggcagaggaagtctgctaacatgcggtgacgtcgaggagaatcctggccca

SEQ ID NO: 148 - DNA, 618 bp, Artificial Sequence
  Synthetic sequence
atgaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctaga

SEQ ID NO: 149 - DNA, 396 bp, Artificial Sequence
  Synthetic sequence
gacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcactgcaggccagccaggcctggcagcccggcgtgtccgccctgaaggaaaaagtaagcgcactgaaagaaaaggtgagcgcgctgaaggagaaagtgagcgccctgaaagagaaagtctctgcccttaaggaggatatc

SEQ ID NO: 150 - DNA, 8961 bp, Artificial Sequence
  Synthetic sequence
ttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagatgaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctagagagggcagaggaagtctgctaacatgcggtgacgtcgaggagaatcctggcccagaattgatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggagggcagaggaagtctgctaacatgcggtgacgtcgaggagaatcctggcccagacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcactgcaggccagccaggcctggcagcccggcgtgtccgccctgaaggaaaaagtaagcgcactgaaagaaaaggtgagcgcgctgaaggagaaagtgagcgccctgaaagagaaagtctctgcccttaaggaggatatcgagcccaggggccccaccatcaagccctgccccccctgcaagtgccccggcttcgccgacctgatgggctacatccccgccgccggcgcccccgccggcggcgccgccagggccctggcccacggcgtgagggtgctggaggacggcgtgaactacgccaccggcaacctgcccggctgcagcttcagcgccctgctgctggccctgctgagctgcctgaccatccccgccagcgcctaataagtttaaacctgctgatcagcctcgactgtgccttctagtcgaccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 151 - DNA, 9084 bp, Artificial Sequence
  Synthetic sequence
ttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttaatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgataagcttgggagttccgcttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctccctatcagtgatagatgaagcttgccaccatgcgcaaaggcgaagaactgtttaccggcattgtgccgattctggtggaactggatggcgatgtgaacggccataaattttttgtgcgcggcgaaggcgaaggcgatgcgaccattggcaaactgagcctgaaatttatttgcaccaccggcaaactgccggtgccgtggccgaccctggtgaccaccctgacctatggcgtgcagtgctttagccgctatccggatcatatgaaacgccatgatttttttaaaagcgcgatgccggaaggctatgtgcaggaacgcaccatttattttaaagatgatggcacctataaaacccgcgcggaagtgaaatttgaaggcgataccctggtgaaccgcattgaactgaaaggcattgattttaaagaagatggcaacattctgggccataaactggaatataactttaacagccataaagtgtatattaccgcggataaacagaacaacggcattaaagcgaactttaccattcgccataacgtggaagatggcagcgtgcagctggcggatcattatcagcagaacaccccgattggcgatggcccggttcttcttcctggcggccgctctagagagggcagaggaagtctgctaacatgcggtgacgtcgaggagaatcctggcccagaattgatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaaggagggcagaggaagtctgctaacatgcggtgacgtcgaggagaatcctggcccagacctgcctgacgaccactacctgtccacccagaccatcctgtccaaggacctgaactccggactcagatctggcagcggtctcgagatggaagttagcgctctggaaaaagaagtgtctgcactcgagaaagaagtaagtgcccttgagaaggaggtgtccgcactcgagaaggaggtcagcgccctggaaaaggaaaagcgagaccatatggttttgcttgagtatgttacagcggctggcattaccgatgcatcactgcaggccagccaggcctggcagcccggcgtgtccgccctgaaggaaaaagtaagcgcactgaaagaaaaggtgagcgcgctgaaggagaaagtgagcgccctgaaagagaaagtctctgcccttaaggaggatatcgagcccaggggccccaccatcaagccctgccccccctgcaagtgccccggcttcgccgacctggtgcccattattgtcgccattgtggccctggtggtcctcattattattgccattgtggtggcgagcattgtgattattgagtaccgcaagattctgcgccagcgcaagattgaccggttgattgaccgcctgattgagcgcgccgaggacgccggcaacgaggctgagggcgaggtgagcgctctggtggagatgggcgtggagatgggccaccacgccccctgggacattgacgacctgctcgagggcggccgctacccatacgacgtcccagactacgctggtaagaaggaccaataataagtttaaacctgctgatcagcctcgactgtgccttctagtcgaccctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatg

SEQ ID NO: 152 - DNA, 177 bp, Artificial Sequence
  Synthetic sequence
atgggctacatccccgccgccggcgcccccgccggcggcgccgccagggccctggcccacggcgtgagggtgctggaggacggcgtgaactacgccaccggcaacctgcccggctgcagcttcagcgccctgctgctggccctgctgagctgcctgaccatccccgccagcgcctaa

SEQ ID NO: 153 - DNA, 300 bp, Artificial Sequence
  Synthetic sequence
gtgcccattattgtcgccattgtggccctggtggtcctcattattattgccattgtggtggcgagcattgtgattattgagtaccgcaagattctgcgccagcgcaagattgaccggttgattgaccgcctgattgagcgcgccgaggacgccggcaacgaggctgagggcgaggtgagcgctctggtggagatgggcgtggagatgggccaccacgccccctgggacattgacgacctgctcgagggcggccgctacccatacgacgtcccagactacgctggtaagaaggaccaataa